SEQUENCE LISTING (14 sequences)

SEQ ID NO: 1 - protein, 18 aa, synthetic construct
EGRGSLLTCGDVEENPGP

SEQ ID NO: 2 - protein, 19 aa, synthetic construct
ATNFSLLKQAGDVEENPGP

SEQ ID NO: 3 - protein, 20 aa, synthetic construct
QCTNYALLKLAGDVESNPGP

SEQ ID NO: 4 - protein, 22 aa, synthetic construct
VKQTLNFDLLKLAGDVESNPGP

SEQ ID NO: 5 - protein, 8 aa, synthetic construct
DXEXNPGP

SEQ ID NO: 6 - protein, 21 aa, synthetic construct
GSGEGRGSLLTCGDVEENPGP

SEQ ID NO: 7 - protein, 22 aa, synthetic construct
GSGATNFSLLKQAGDVEENPGP

SEQ ID NO: 8 - protein, 23 aa, synthetic construct
GSGQCTNYALLKLAGDVESNPGP

SEQ ID NO: 9 - protein, 25 aa, synthetic construct
GSGVKQTLNFDLLKLAGDVESNPGP

SEQ ID NO: 10 - protein, 25 aa, synthetic construct
GGGGSGGGGSGGGGSGGGGSGGGGS

SEQ ID NO: 11 - protein, 850 aa, synthetic construct
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWEGGGGSGGGGSGGGGSGGGGSGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 12 - protein, 879 aa, synthetic construct
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWEELSGSGEGRGSLLTCGDVEENPGPLRSPGRVNMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 13 - protein, 874 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGEGRGSLLTCGDVEENPGPLRSPVTMDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 14 - protein, 5 aa, synthetic construct
GGGGS

(19) United States
(12) Patent Application Publication
(10) Pub. No.: US 2026/0207787 A1
(43) Pub. Date: Jul. 23, 2026

(54) IMMUNE ACTIVATION

(71) Applicant: The Johns Hopkins University, Baltimore, MD (US)

(72) Inventors: Jordan J. Green, Woodstock, MD (US); Joanna Yuan Shin Yang, Houston, TX (US); Stephany Yi Tzeng, Baltimore, MD (US); Jamie Spangler, Baltimore, MD (US)

(21) Appl. No.: 19/455,150

(22) Filed: Jan. 21, 2026

Related U.S. Application Data
(60) Provisional application No. 63/747,964, filed on Jan. 22, 2025.
(60) Provisional application No. 63/862,349, filed on Aug. 12, 2025.

(51) Int. Cl. A61K 48/00; A61K 38/00; C07K 14/47; C07K 14/525; C07K 14/54; C12N 15/85

(52) U.S. Cl. CPC A61K 48/0066; A61K 48/0041; C07K 14/47; C07K 14/525; C07K 14/5434; C12N 15/85; A61K 38/00; C07K 2319/02; C12N 2840/20

(57) ABSTRACT

Provided herein is technology relating to therapies for activating a human immune system and particularly, but not exclusively, to polypeptide constructs designed to co-express costimulatory surface proteins (signal 2) and secreted cytokines (signal 3), nucleic acids encoding the polypeptide constructs, cells expressing the polypeptide constructs, and methods of using the polypeptide constructs and related nucleic acids and cells to reprogram cells into antigen presenting cells. The technology finds use in the treatment of human diseases that can have an immune system component (e.g., cancer, infectious diseases, and autoimmune diseases).

This application claim priority to provisional patent applications 63/747,964, filed Jan. 22, 2025 and 63/862,349, filed Aug. 12, 2025; each of which is herein incorporated by reference in their entireties.

### STATEMENT REGARDING FEDERALLY SPONSORED RESEARCH OR DEVELOPMENT

This invention was made with government support under EB028239, awarded by the National Institutes of Health. The government has certain rights in the invention.

### SEQUENCE LISTING

The text of the computer readable sequence listing filed herewith, titled “44051-203_SEQUENCE_LISTING”, created Jan. 21, 2026, having a file size of 15,796 bytes, is hereby incorporated by reference in its entirety.

### FIELD

Provided herein is technology relating to therapies for activating a human immune system and particularly, but not exclusively, to polypeptide constructs designed to co-express costimulatory surface proteins (signal 2) and secreted cytokines (signal 3), nucleic acids encoding the polypeptide constructs, cells expressing the polypeptide constructs, and methods of using the polypeptide constructs and related nucleic acids and cells to reprogram cells to function as antigen presenting cells. The technology finds use in the treatment of human diseases that have an immune system component (e.g., cancer, infectious diseases, and autoimmune diseases).

### BACKGROUND

Dendritic cells play a role in the immune system response to disease, primarily transforming naïve T cells to an activated state by presenting the T cells with antigens (signal 1), costimulatory surface proteins (signal 2), and secreted cytokines (signal 3). See, e.g., Tai (2018) “Molecular mechanisms of T cells activation by dendritic cells in autoimmune diseases” Front Pharmacol 9:642, incorporated herein by reference. Previous studies indicated that delivering signal 2 (e.g., 4-1BBL) and signal 3 (e.g., IL12) co-stimulatory molecules using nanoparticles induced anti-tumor immune responses by reprograming tumor cells into tumor-associated antigen presenting cells. Chen (2024) “CD8+ T cell-based cancer immunotherapy” J Transl Med 22 (1): 394, incorporated herein by reference. See, e.g., Int'l Pat. App. Pub. No. WO 2020/198145, which is incorporated herein by reference. However, the present technologies involve use of multiple genetic constructs, which imposes challenges for clinical use.

### SUMMARY

Accordingly, provided herein are immunotherapeutic technologies that provide the signal 2 and signal 3 transgenes on a single nucleic acid construct. In particular, two different types of bicistronic nucleic acid constructs are provided: 1) a first type expresses signal 2 and signal 3 polypeptides linked by an auto catalytic linker; and 2) a second type expresses signal 2 and signal 3 polypeptides linked by a polymeric linker (“tether”). Data collected during the development of the technology described herein indicated that cells comprising the bicistronic constructs expressed both signal 2 and signal 3 gene products and induced an immune response in co-cultured cells. Embodiments of the technology provided herein find use in clinical treatment of disease by increasing the co delivery of both signal 2 and signal 3 immunomodulatory signals to the same tumor cells, thus enhancing therapeutic efficacy. Furthermore, the technology described herein provides improvements over conventional technologies by simplifying manufacturing processes and thus facilitating scalability; and by improving access to successful regulatory approval by minimizing the complexity of gene therapy formulations.

For example, in some embodiments, the technology provides a composition comprising a nucleic acid comprising a first nucleotide sequence encoding a signal 2 polypeptide and a second nucleotide sequence encoding a signal 3 polypeptide. As described herein, the signal 2 transgene and the signal 3 transgene are provided on a single (i.e., the same) nucleic acid construct. That is, one nucleic acid construct comprises both the signal 2 transgene and the signal 3 transgene. In some embodiments, the first nucleotide sequence is upstream of the second nucleotide sequence. In some embodiments, the second nucleotide sequence is upstream of the first nucleotide sequence. In some embodiments, the composition further comprises a nucleotide sequence encoding a linker between the first nucleotide sequence and the second nucleotide sequence. In some embodiments, the linker is a 2A peptide. In some embodiments, the linker is T2A, P2A, E2A, or F2A. In some embodiments, the linker further comprises the amino acid sequence GSG at the N-terminus. In some embodiments, the composition further comprises a nucleotide sequence encoding a tether between the first nucleotide sequence and the second nucleotide sequence. In some embodiments, the tether comprises the amino acid sequence GGGGS (SEQ ID NO: 14). In some embodiments, the tether comprises the amino acid sequence (GGGGS)n (SEQ ID NO: 14), where n=2 to 10. In some embodiments, the nucleic acid is a plasmid. In some embodiments, the nucleic acid is an antibiotic resistance gene-free plasmid. In some embodiments, the nucleic acid comprises a bicistronic gene comprising the first nucleotide sequence and the second nucleotide sequence. In some embodiments, the signal 2 polypeptide is a cell surface bound protein that regulates immune cells. In some embodiments, the signal 3 polypeptide is a secreted protein that regulates immune cells. In some embodiments, the signal 3 polypeptide comprises a cytokine. In some embodiments, the cytokine comprises an interleukin. In some embodiments, the first nucleotide sequence encodes 4-1BBL, CD28, CD80, CD86, OX40L, or GITRL. In some embodiments, the second nucleotide sequence encodes TGF-ß1, TGF-ß2, TGF-ß3, TGF-4, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IFN-α, or IFN-ß.

In some embodiments, the technology provides a gene delivery formulation comprising polymers and a composition described herein (e.g., a composition comprising a nucleic acid comprising a first nucleotide sequence encoding a signal 2 polypeptide and a second nucleotide sequence encoding a signal 3 polypeptide). In some embodiments, the gene delivery formulation comprises a composition described herein (e.g., a composition comprising a nucleic acid comprising a first nucleotide sequence encoding a signal 2 polypeptide and a second nucleotide sequence encoding a signal 3 polypeptide) and a biomaterial. As described herein, the signal 2 transgene and the signal 3 transgene are provided on a single (i.e., the same) nucleic acid construct. That is, one nucleic acid construct comprises both the signal 2 transgene and the signal 3 transgene. In some embodiments, the biomaterial comprises a cationic biodegradable polymer. In some embodiments, the cationic biodegradable polymer is poly(beta-amino ester) (PBAE). In some embodiments, the gene delivery formulation is in the form of nanoparticles.

Further embodiments provide a composition comprising a polypeptide comprising a signal 2 immune co-stimulatory molecule amino acid sequence and a signal 3 soluble molecule amino acid sequence. In some embodiments, the signal 2 co-stimulatory protein is expressed on the surface of a cell. In some embodiments, the signal 2 co-stimulatory protein regulates immune cells. In some embodiments, the signal 3 soluble molecule is a secreted protein that regulates immune cells. In some embodiments, the signal 3 soluble molecule comprises a cytokine. In some embodiments, the cytokine comprises an interleukin. In some embodiments, the composition further comprises a linker amino acid sequence between the signal 2 co-stimulatory molecule amino acid sequence and the signal 3 soluble molecule amino acid sequence. In some embodiments, the linker amino acid sequence is a 2A peptide. In some embodiments, the linker amino acid sequence is T2A, P2A, E2A, or F2A (SEQ ID NO: 1-4). In some embodiments, the linker amino acid sequence further comprises the amino acid sequence GSG at the N-terminus. In some embodiments, the composition further comprises a tether amino acid sequence between the signal 2 co-stimulatory molecule amino acid sequence and the signal 3 soluble molecule amino acid sequence. In some embodiments, the tether amino acid sequence comprises the amino acid sequence GGGGS (SEQ ID NO: 14). In some embodiments, the tether amino acid sequence comprises the amino acid sequence (GGGGS)n (SEQ ID NO: 14), where n=2 to 10. In some embodiments, the signal 2 co-stimulatory molecule is 4-1BBL, CD28, CD80, CD86, OX40L, or GITRL. In some embodiments, the signal 3 soluble molecule is TGF-ß1, TGF-ß2, TGF-ß3, TGF-ß4, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IFN-α, or IFN-ß.

Some embodiments provide a method for reprogramming one or more cancer cells into one or more tumor-derived antigen-presenting cells (tAPCs). In some embodiments, the one or more tAPCs mimic a natural antigen presenting cell (APC) and direct an immune response against themselves and other cancer cells. For instance, in some embodiments, methods comprise transfecting the one or more cancer cells with a composition as described herein (e.g., a composition comprising a nucleic acid comprising a first nucleotide sequence encoding a signal 2 polypeptide and a second nucleotide sequence encoding a signal 3 polypeptide). As described herein, the signal 2 transgene and the signal 3 transgene are provided on a single (i.e., the same) nucleic acid construct. That is, one nucleic acid construct comprises both the signal 2 transgene and the signal 3 transgene. In some embodiments, transfecting the one or more cancer cells promotes an immune cell activation against one or more antigens expressed on the one or more cancer cells. In some embodiments, the one or more tAPCs activate an antigen-specific T-cell response against MHC 1+ tumor cells. In some embodiments, the one or more tAPCs provide an activating signal to one or more natural killer (NK) cells to induce anti-tumor cytotoxicity therein. In some embodiments, the one or more tAPCs activate an antigen-independent NK cell response against MHC I-/low tumor cells. In some embodiments, methods further comprise inducing a systemic immune response resulting in cell death of distant metastases.

Some embodiments provide methods of treating a cancer. For instance, in some embodiments, methods comprise administering to a subject in need of treatment a composition as described herein (e.g., a composition comprising a nucleic acid comprising a first nucleotide sequence encoding a signal 2 polypeptide and a second nucleotide sequence encoding a signal 3 polypeptide). As described herein, the signal 2 transgene and the signal 3 transgene are provided on a single (i.e., the same) nucleic acid construct. That is, one nucleic acid construct comprises both the signal 2 transgene and the signal 3 transgene. In some embodiments, the cancer is selected from the group consisting of a melanoma, a breast cancer, a colorectal cancer, a liver cancer, and a brain cancer.

Embodiments further provide a pharmaceutical formulation comprising a composition as described herein (e.g., a composition comprising a nucleic acid comprising a first nucleotide sequence encoding a signal 2 polypeptide and a second nucleotide sequence encoding a signal 3 polypeptide) and a pharmaceutically acceptable carrier. As described herein, the signal 2 transgene and the signal 3 transgene are provided on a single (i.e., the same) nucleic acid construct. That is, one nucleic acid construct comprises both the signal 2 transgene and the signal 3 transgene.

Embodiments provide a kit comprising a composition as described herein (e.g., a composition comprising a nucleic acid comprising a first nucleotide sequence encoding a signal 2 polypeptide and a second nucleotide sequence encoding a signal 3 polypeptide). As described herein, the signal 2 transgene and the signal 3 transgene are provided on a single (i.e., the same) nucleic acid construct. That is, one nucleic acid construct comprises both the signal 2 transgene and the signal 3 transgene.

Some embodiments provide use of a composition as described herein (e.g., a composition comprising a nucleic acid comprising a first nucleotide sequence encoding a signal 2 polypeptide and a second nucleotide sequence encoding a signal 3 polypeptide) in combination with one or more anti cancer immune checkpoint inhibitor molecules. As described herein, the signal 2 transgene and the signal 3 transgene are provided on a single (i.e., the same) nucleic acid construct. That is, one nucleic acid construct comprises both the signal 2 transgene and the signal 3 transgene. In some embodiments, the checkpoint inhibitor molecule comprises an anti-PD-1 antibody.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A is a schematic drawing of a plasmid comprising a nucleotide sequence encoding signal 2 (e.g., 4-1BBL), a T2A peptide linker, and signal 3 (e.g., IL-12). Signal 2 is upstream of signal 3.

FIG. 1B is a schematic drawing of a plasmid comprising a nucleotide sequence encoding signal 3 (e.g., IL-12), a T2A peptide linker, and signal 2 (e.g., 4-1BBL). Signal 3 is upstream of signal 2.

FIG. 1C is a schematic drawing of a plasmid comprising a nucleotide sequence encoding signal 2 (e.g., 4-1BBL) a tether linker (e.g., comprising a repeated sequence of GGGGS (SEQ ID NO: 14)), and signal 3 (IL-12).

FIG. 2 is a bar plot showing immune activation by construct 41BBL T2A IL12 (i.e., a “skip” approach in which the nucleic acid construct expresses two separate signal 2 (1BBL) and signal 3 (IL12) proteins from the same DNA molecule) and construct 1BBL G4S IL12 (i.e., a protein “fusion” approach using an amino acid tether of glycine and serine linking the signal 2 (1BBL) and signal 3 (IL12) proteins for expression as a single polypeptide). Immune activation was measured using IFN-gamma levels.

FIG. 3A is a bar plot of antibody staining for 4-1BBL to measure 4-1BBL expression.

FIG. 3B is a bar plot showing IL12 expression as measured by ELISA after dual transgene construct delivery.

FIG. 3C is a bar plot showing the results of an ELISA assay to measure IFN-gamma.

FIG. 3D is a bar plot showing the results of an ELISA assay to measure IFN-gamma in experiments in which doses of dual transgene DNA constructs were varied to improve IFN-gamma expression.

FIG. 3E is a bar plot showing IL12 ELISA data of cell media and of cells after lysis of tethered dual transgene constructs comprising human 41BBL and IL12.

FIG. 3F is a bar plot showing IL12 ELISA data of cell media and of cells after lysis of tethered dual transgene constructs comprising mouse 41BBL and IL12.

FIG. 4A to 4I show in vitro validation of delivery and function of bicistronic plasmids. (FIG. 4A) Mouse 4-1BBL and mouse IL-12 were assembled onto a single plasmid separated with 2A peptide. (FIG. 4B) Mouse 4-1BBL and mouse IL-12 were assembled onto a single plasmid separated by a (G4S)n linker. (FIG. 4C) B16-F10 cells were transfected with bicistronic plasmids as shown in the experimental timeline. (FIG. 4D) 4-1BBL expression levels after co-delivery of 4-1BBL and IL-12 as separate plasmids or bicistronic plasmids as determined by flow cytometry (one-way ANOVA, Dunnett's test, compared to 4-1BBL/IL-12 as separate plasmids). (FIG. 4E) Surface IL-12 expression after co-delivery of 4-1BBL and IL-12 as separate plasmids or bicistronic plasmids as determined by flow cytometry (one-way ANOVA, Dunnett's test, compared to 4-1BBL/IL-12 as separate plasmids). (FIG. 4F) IL-12 expression in cell culture supernatant after co delivery of 4-1BBL and IL-12 as separate plasmids or bicistronic plasmids as determined by enzyme-linked immunoassay (ELISA) (one-way ANOVA, Dunnett's test, compared to 4-1BBL/IL-12 as separate plasmids). (FIG. 4G) B16-F10 cells were reprogrammed and co-cultured with splenocytes. (FIG. 4H) Comparison of IFNγ expression in cell culture supernatant between plasmids as determined by ELISA (one-way ANOVA, Dunnett's test, compared to 4-1BBL/IL-12 as separate plasmids). (FIG. 41) Comparison of IFNγ expression between plasmids with differing lengths of the (G4S)n tether as determined by ELISA (one-way ANOVA, Dunnett's test, compared to 4G512. Significance in FIG. 4A to 4I is represented by *p≤0.05, **p≤0.01, ***p≤0.001, and ****p<0.0001. Each data bar represents means±standard error of the mean with four technical replicates.

FIG. 5A to 5C show in vitro delivery efficiency for various PBAE polymers. (FIG. 5A) Transfection of B16-F10 cells by seven PBAE polymers, as determined by flow cytometry. (FIG. 5B) Mean fluorescence intensity of PBAE polymers after transfection are shown. (FIG. 5C) B16-F10 cell viability after transfection with PBAE polymers.

FIG. 6A to I show survival of B16-F10 tumor-bearing mice after intratumoral injection of 4-1BBL- and IL-12-loaded NPs or recombinant rIL-12. (FIG. 6A) Experimental timeline of mice inoculated with B16-F10 tumors is shown (FIG. 6B) Growth curves of B16-F10 tumors treated with systemic recombinant IL-12 (rIL-12), luciferase NPs, 4-1BBL/IL-12 NPs, 4G512 NPs, or 4T12 NPs (two-way ANOVA, Dunnett's test, compared to 4-1BBL/IL-12 treated mice). Each data bar represents mean±SEM with eight replicates. (FIG. 6C) Survival curves for mice treated with rIL-12, luciferase NPs, or tAPC NPs (log-rank test). (FIG. 6D to FIG. 61) Tumor growth curves for each treatment group. Data points represent mean±SEM with four technical replicates. Significance in FIG. 6A to 6I is represented by *p≤0.05, **p≤0.01, ***p≤0.001, and ****p≤0.0001.

FIG. 7A to 7E show characterization of nanoparticle formulations for in vivo delivery. (FIG. 7A) Nanoparticles were ~200 nm in hydrodynamic diameter (One-way ANOVA, Dunnett's test, compared to 4-1BBL/IL-12). (FIG. 7B) Polydispersity indices are shown (One-way ANOVA, Dunnett's test, compared to 4-1BBL/IL-12). (FIG. 7C) Zeta potentials of nanoparticles are neutral to negative (One-way ANOVA, Dunnett's test, compared to 4-1BBL/IL-12). (FIG. 7D) TEM imaging of dried NPs formed with two separate plasmids for 4-1BBL and IL-12. (FIG. 7E) TEM imaging of dried NPs formed with a bicistronic plasmid encoding both 4-1BBL and IL-12. Each data bar represents means #standard error of the mean with four technical replicates. Significance in FIG. 7A to 7E is represented by *p≤0.05, **p≤0.01, ***p≤0.001, and ****p≤0.0001.

FIG. 8 shows immunohistochemistry staining of harvested B16-F10 tumors reveals immune infiltration. Tumor tissue extracted from mice treated systemically with 3 ng/kg or 500 ng/kg of recombinant IL-12 or intratumorally with luciferase NPs, co-delivered 4-1BBL and IL12 plasmid NPs, 4G512 NPs or 4T12 NPs. B16-F10 cells are shown in beige brown. CD8+ cells are stained purple, CD4+ cells stained teal, and FOXP3+ cells stained dark brown.

FIG. 9A to 9F show data collected for tumor rechallenge experiments to assess durability of tAPC NP-induced anti-tumor effects. (FIG. 9A) Long-term survivors were rechallenged with B16-F10 tumors on day 100 alongside age-matched naïve mice (two-way ANOVA, Dunnett's test, compared to naïve mice). (FIG. 9B) Average size new tumor formation in naïve mice compared to 4-1BBL/IL-12-, 4G512- or 4T12-treated mice. Individual tumor growths in (FIG. 9C) naïve mice, (FIG. 9D) 4-1BBL/IL-12 NP-treated mice, (FIG. 9E) 4G512 NP-treated mice, and (FIG. 9F) 4T12 NP-treated mice.

FIG. 10A to FIG. 10F show survival in a B16-F10 mouse model with antibiotic resistance-gene free Nanoplasmids. (FIG. 10A) Transfection of B16-F10 cells using pUNO1 vs Nanoplasmid backbones as determined by flow cytometry. (FIG. 10B) GFP expression as determined by flow cytometry. (FIG. 10C) Average tumor growth of tumors treated with tAPC NPs vs luciferase NPs. (FIG. 10D) Average tumor growth of tumors treated with tAPC NPs+anti-PD1 vs luciferase NPs+anti-PD1. (FIG. 10E) Survival curves of mice treated with tAPC NPs vs luciferase NPs. (FIG. 10F) Survival curves of mice treated with tAPC NPs+anti-PD1 vs luciferase NPs+anti-PD1. Each data bar represents means±standard error of the mean with four technical replicates. Significance in FIG. 10A to 10F is represented by *p≤0.05, **p≤0.01, ***p≤0.001, and ****p≤0.0001.

FIG. 11A to 11I show In vitro validation of delivery and function of bicistronic plasmids. (A) Mouse 4-1BBL and mouse IL-12 were assembled onto a single plasmid separated with 2A peptide. (B) Mouse 4-1BBL and mouse IL-12 were assembled onto a single plasmid separated by a (G4S)n linker. (C) B16-F10 cells were transfected with bicistronic plasmids as shown in the experimental timeline. (D) 4-1BBL expression levels after co-delivery of 4-1BBL and IL-12 as separate plasmids or bicistronic plasmids as determined by flow cytometry (one-way ANOVA, Dunnett's test, compared to 4-1BBL/IL-12 as separate plasmids). (E) Surface IL-12 expression after co-delivery of 4-1BBL and IL-12 as separate plasmids or bicistronic plasmids as determined by flow cytometry (one-way ANOVA, Dunnett's test, compared to 4-1BBL/IL-12 as separate plasmids). (F) IL-12 expression in cell culture supernatant after co-delivery of 4-1BBL and IL-12 as separate plasmids or bicistronic plasmids as determined by Enzyme-Linked Immunosorbent Assay (ELISA) (one-way ANOVA, Dunnett's test, compared to 4-1BBL/IL-12 as separate plasmids). (G) B16-F10 cells were reprogrammed and co-cultured with splenocytes. (H) Comparison of IFNγ expression in cell culture supernatant between plasmids as determined by ELISA (one-way ANOVA, Dunnett's test, compared to 4-1BBL/IL-12 as separate plasmids). (I) Comparison of IFNγ expression between plasmids with differing lengths of the (G4S)n tether as determined by ELISA (one-way ANOVA, Dunnett's test, compared to 4G₅12). Significance is represented by *p≤0.05, **p≤0.01, ***p≤0.001, and ****p≤0.0001. Each data bar represents mean±standard error of the mean (SEM) with four biological replicates.

FIG. 12A-12I shows that B16-F10 tumor-bearing mice survived longer after intratumoral injection of 4-1BBL- and IL-12-loaded NPs. (A) Experimental timeline of mice inoculated with B16-F10 tumors is shown (B) Growth curves of B16-F10 tumors treated with systemic recombinant IL-12 (rIL-12), luciferase (fLuc) NPs, 4-1BBL/IL-12 NPs, 4G₅12 NPs, or 4T12 NPs (two-way ANOVA, Dunnett's test, compared to 4-1BBL/IL-12-treated mice). Each data bar represents mean±SEM with eight biological replicates. (C) Survival curves for mice treated with rIL-12, luciferase NPs, or tAPC NPs (log-rank test with Bonferroni correction for multiple comparisons). (D to I) Tumor growth curves for each treatment group. Data points represent mean±SEM with eight biological replicates. Significance is represented by *p≤0.05, **p≤0.01, ***p≤0.001, and ****p≤0.0001.

FIG. 13 shows Immunohistochemistry staining of harvested B16-F10 tumors reveals immune infiltration.

FIG. 14A-14F shows a tumor rechallenge to assess durability of tAPC NP-induced anti-tumor effects. (A) Long-term survivors were rechallenged with B16-F10 tumors on day 100 alongside age-matched naïve mice (two-way ANOVA, Dunnett's test, compared to naïve mice). (B) Average size of new tumors in naïve mice compared to 4-1BBL/IL-12-, 4G₅12- or 4T12-treated mice. Individual tumor growth in (C) naïve mice (n=3), (D) 4-1BBL/IL-12 NP-treated mice (n=3), (E) 4G₅12 NP-treated mice (n=2), and (F) 4T12 NP-treated mice (n=2). Data points represent mean±SEM. Significance is represented by *p≤0.05, **p≤0.01, ***p≤0.001, and ****p≤0.0001.

FIG. 15A-15C shows survival in a B16-F10 mouse model with antibiotic-gene free Nanoplasmids. (A) Transfection of B16-F10 cells using 600 ng of pUNO1 or Nanoplasmid (NanoP) backbones as determined by flow cytometry. (B) GFP expression, determined by flow cytometry. Each data bar represents mean±SEM with four biological replicates. (C) Average tumor growth of tumors treated with Nanoplasmid 4T12 NPs+anti-PD1 vs Nanoplasmid luciferase NPs+anti-PD1. Each data curve represents eight biological replicates. Significance is represented by *p≤0.05, **p≤0.01, ***p≤0.001, and ****p≤0.0001.

FIG. 16 shows that bicistronic IL-12 and 4-1BBL plasmid DNA result in tumor reprogramming into tumor-associated antigen presenting cells (tAPC) and stimulate an anti-tumor response.

FIG. 17A-17F shows In vitro delivery efficiency for various PBAE polymers. (A) PBAE synthesis via Michael addition reaction. (B) PBAE monomer structures. (C) PBAE 4-4-6 and 5-3-6 structures. (D) Transfection of B16-F10 cells by seven PBAE polymers, as determined by flow cytometry. (E) Normalized GFP mean fluorescence intensity after transfection with PBAE NPs. (F) B16-F10 cell viability after transfection with PBAE polymers, normalized to untreated. Each data bar represents means±standard error of the mean with four biological replicates.

FIG. 18A-18E shows characterization of nanoparticle formulations for in vivo delivery. (A) Nanoparticles were measured via DLS (One-way ANOVA, Dunnett's test, compared to 4-1BBL/IL-12). (B) Polydispersity indices are shown (One-way ANOVA, Dunnett's test, compared to 4-1BBL/IL-12). (C) Zeta potentials of nanoparticles shown (One-way ANOVA, Dunnett's test, compared to 4-1BBL/IL-12). (D) TEM imaging of dried NPs formed with two separate plasmids for 4-1BBL and IL-12. (E) TEM imaging of dried NPs formed with a bicistronic plasmid encoding both 4-1BBL and IL-12. Each data bar represents means±standard error of the mean with four technical replicates. Significance is represented by *p≤0.05, **p≤0.01, ***p≤0.001, and ****p≤0.0001.

FIG. 19A-19D shows survival in B16-F10 mouse model with antibiotic-gene free Nanoplasmids delivering 4-1BBL and IL-12 on separate plasmids. (A) Average tumor growth of tumors treated with tAPC NPs vs luciferase NPs (Two-way ANOVA, Dunnett's test, compared to control). (B) Average tumor growth of tumors treated with tAPC NPs+anti-PD1 vs luciferase NPs+anti-PD1 (Two-way ANOVA, Dunnett's test, compared to control). (C) Survival curves of mice treated with tAPC NPs vs luciferase NPs. (D) Survival curves of mice treated with tAPC NPs+anti-PD1 vs luciferase NPs+anti-PD1. Each data bar represents mean±SEM with four biological replicates. Significance is represented by *p≤0.05, **p≤0.01, ***p≤0.001, and ****p≤0.0001.

FIG. 20A-20B shows tumor growth curves in B16-F10 mouse model comparing Nanoplasmid fLuc to Nanoplasmid. (A) Average tumor growth in mice treated with Nanoplasmid fLuc NPs+anti-PD1 vs Nanoplasmid 4T12 NPs+anti-PD1 (Two-way ANOVA, Sidak's test). (B) Individual tumor growth in mice treated with Nanoplasmid fLuc NPs+anti-PD1 vs Nanoplasmid 4T12 NPs+anti-PD1. Each data bar represents mean #SEM with eight biological replicates. Significance is represented by *p≤0.05, **p≤0.01, ***p≤0.001, and ****p≤0.0001.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

T-cell activation requires the coordination of three signals between T cells and antigen presenting cells (APCs). Signal 1 is provided by T-cell receptor interacting with an MHC:antigen complex. Signal 2 is provided by co-stimulatory receptors interacting with their respective ligands. Signal 3 is provided by secreted cytokine.

Prior research has demonstrated that poly(8-amino ester) nanoparticle (PBAE NP)-mediated co-delivery of plasmids encoding 4-1BBL (signal 2) and IL-12 (signal 3) can reprogram tumor cells into tumor-associated antigen presenting cells (tAPCs) and generate an anti-tumor immune response. This approach has been validated in a variety of solid tumors, including melanoma, Merkel cell carcinoma, colorectal carcinoma, and breast cancer.

However, translating gene therapy strategies into the clinic requires improving the scalability and safety profiles of genetic constructs. Current dual-signal delivery methods comprise co-delivering two separate genetic constructs: one plasmid DNA encoding 4-1BBL; and a second plasmid DNA encoding IL-12. Preparing clinical grade plasmid DNA comprises amplifying and purifying each plasmid, and a dual-plasmid system effectively doubles the DNA manufacturing cost compared to a single-plasmid system. Furthermore, the presence of antibiotic resistance genes in plasmids may also elicit regulatory and safety concerns for clinical applications. To address these translational barriers, the technology described herein provides several bicistronic versions of 4-1BBL and IL-12.

In particular, the technology described herein combines two signals onto a single plasmid using two methods: 1) a self-cleaving 2A sequence; and 2) a Glycine-Glycine-Glycine-Glycine-Serine (G4S)n peptide tether. In the 2A format, 4-1BBL and IL-12 are transcribed from a single plasmid and form two distinct proteins. In the (G4S)n format, the normally secreted IL-12 is tethered to the surface-bound 4-1BBL as a fusion protein. 2A peptides have been widely explored to express recombinant proteins in a multi-cistronic manner, such as expressing the four CD3 protein subunits to rescue T-cell function. 2A peptides have also been utilized to express both the heavy and light chains of an antibody ex vivo from the same plasmid. GS linkers have been widely explored in protein engineering as flexible linkers to connect protein domains. Embodiments of the technology described herein use (Gly-Gly-Gly-Gly-Ser)n, or (G4S)n , for the tethering of IL-12 to 4-1BBL15 because this linker has been validated extensively in both research and pharmaceutical applications.

Using a linker to tether IL-12 to 4-1BBL provides two advantages: 1) ensuring IL-12 remains local to the tumor due to tethering to the cell surface; and 2) enhancing T-cell activation by increasing the effective concentration of 4-1BBL and IL-12 through their spatial proximity.

Another barrier to clinical translation for many research grade non viral gene therapies, including the current tAPC approach, is the presence of antibiotic resistance genes in the plasmids used for gene therapy. These antibiotic resistance genes pose a risk of horizontal transfer to bacterial populations in the human gut microbiome. Thus, removing antibiotic resistance genes from plasmid systems will help prevent the spread of antibiotic resistance, avoid potential allergic reactions in patients, and streamline compliance with good manufacturing process (GMP). To eliminate the antibiotic resistance gene from the plasmid backbone, the Nanoplasmid Vector System from Aldevron, which has already been utilized in several clinical trials was used.

Taken together, the improved plasmids and methods of use described herein overcome critical barriers to clinical translation of DNA tAPC NPs and other co-delivery-based therapeutic strategies by streamlining the manufacturing pipeline and improving the regulatory readiness of the technology. Coupled with nanoparticle-mediated administration, the bicistronic, antibiotic-free plasmid design strategy offers a potent and localized immunomodulatory effect with the potential for improved safety and translational feasibility of nonviral gene therapy approaches.

Accordingly, provided herein is technology relating to therapies for activating a human immune system and particularly, but not exclusively, to polypeptide constructs designed to co-express costimulatory surface proteins (signal 2) and secreted cytokines (signal 3), nucleic acids encoding the polypeptide constructs, cells expressing the polypeptide constructs, and methods of using the polypeptide constructs and related nucleic acids and cells to reprogram cells to function as antigen presenting cells. The technology finds use in the treatment of human diseases that have an immune system component (e.g., cancer, infectious diseases, and autoimmune diseases).

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “.free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the term “number” shall mean one or an integer greater than one (e.g., a plurality). As used herein, the term “plurality” shall mean more than one.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre determined threshold. The pre determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

A T cell or T lymphocyte is a cell that belongs to a group of white blood cells known as lymphocytes and plays a central role in cell-mediated immunity. Different types of T cells include, but are not limited to, T helper cells, cytotoxic T cells, memory T cells, regulatory T cells (also known as suppressor cells), and natural killer T cells. A T cell can be distinguished from other lymphocytes by the presence of a T cell receptor (TCR) on its cell surface. A T cell receptor is a protein that is found on the surface of a T cell and it is responsible for recognizing antigens bound to MHC molecules. This recognition ensures that only a T cell with a TCR specific to a particular antigen is activated. In some embodiments, the interaction of the TCR with a MHC:antigen complex is the first signal in the activation or modulation of a T cell. The antigen can be presented to the T cell by a MHC-dimer or -tetramer molecule. The MHC-dimer or -tetramer molecule can be easily loaded with any MHC-restricted peptide of interest. By loaded, it is meant that the peptide is attached in some way to the MHC-dimer or -tetramer, whether by covalent interactions or by noncovalent interactions or both.

As used herein, the term “naïve T cell” refers to a T cell that can respond to a novel pathogen that the immune system has not yet encountered. Recognition by a naïve T cell of its cognate antigen results in the initiation of an immune response, which may include modulation of a T cell and/or proliferation of the T cell.

As used herein, “contacting” or “interacting” means any action that results in at least one molecule affecting another molecule, either by physically contacting, or by getting in close enough proximity that one molecule affects the actions of another molecule. For example, the TCR of a T cell may interact with a MHC:antigen complex in such a way that the antigen of the MHC:antigen complex activates the T cell without physically binding to the TCR. In another example, the antigen physically contacts the TCR.

As used herein, the term “co-stimulatory signal” refers to a signal that leads to T cell proliferation and/or upregulation or downregulation of key molecules. As used herein, the term “co-stimulatory molecule” refers to a molecule on an antigen presenting cell that specifically binds a cognate co-stimulatory molecule on a T cell, thereby providing a signal which mediates a T cell response, including, but not limited to, proliferation, activation, differentiation, and the like. A co-stimulatory molecule can include, but is not limited to, anti-CD28, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory molecule may also be an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

As used herein, the term “cancer” refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells are in the form of a tumor; such cells may exist locally within a subject or circulate in the blood stream as independent cells, for example, leukemic cells. A cancer can include, but is not limited to, acute lymphocytic leukemia, acute myelogenous leukemia, angiosarcoma, basal cell carcinoma, bladder cancer, brain cancer (e.g., gliomas), breast cancer, cervical cancer, choriocarcinoma, colon cancer, colorectal cancer, corpus uteri cancer, endocrine cancer, esophageal cancer, Ewing's Sarcoma, eye or ocular cancer, gastrointestinal cancer, head cancer, head and neck cancer, hemangioendothelioma, hemangiomas, hepatocellular carcinoma (HCC), Kaposi's Sarcoma, larynx cancer, leukemia/lymphoma, liver cancer, lung cancer, lymphoma, lymphangiogenesis, melanoma, mouth/pharynx cancer, neck cancer, neuroblastoma, neurofibromatosis, oral cancer, ovarian cancer, pancreatic cancer, prostate cancer, rectal cancer, renal cancer, rhabdomyosarcoma, stomach cancer, skin cancer, small cell lung cancer, squamous cell carcinoma, testicular cancer, throat cancer, tuberous sclerosis, urinary cancer, uterine cancer, Wilms Tumor, benign and malignant tumors, and adenomas. In particular embodiments, the cancer is selected from the group consisting of a melanoma, a breast cancer (including triple-negative breast cancer), colorectal cancer, liver cancer, and brain cancer (including a glioma).

As used herein, the term “treating” refers to reversing, alleviating, inhibiting the progression of, preventing or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the therapeutic compositions and methods can be used prophylactically to prevent or reduce the incidence or recurrence of the disease, disorder, or condition.

As used herein, the term “inhibit” and grammatical derivations thereof refer to the ability of a therapeutic composition or method described herein to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

As used herein, the term “effective amount” of a therapeutic composition refers to the amount necessary to elicit a desired biological response. As will be appreciated by those of ordinary skill in this art, the effective amount of a therapeutic composition may vary depending on such factors as the desired biological endpoint, the agent to be delivered, the makeup of the pharmaceutical composition, the target tissue, and the like.

As used herein, the term “combination” is used in its broadest sense and means that a subject is administered at least two agents, more particularly a therapeutic as described herein and at least one second therapeutic agent. More particularly, the term “in combination” refers to the concomitant administration of two (or more) therapeutic agents for the treatment of, e.g., a single disease state. As used herein, the therapeutic agents may be combined and administered in a single dosage form, may be administered as separate dosage forms at the same time, or may be administered as separate dosage forms that are administered alternately or sequentially on the same or separate days. In some embodiments, the therapeutic agents are combined and administered in a single dosage form. In some embodiments, the therapeutic agents are administered in separate dosage forms (e.g., wherein it is desirable to vary the amount of one but not the other). The single dosage form may include additional therapeutic and/or active agents for the treatment of the disease state.

As used herein, the term “nanoparticle” refers to a particle having at least one dimension in the range of approximately 1 nm to approximately 1000 nm, including any integer value between 1 nm and 1000 nm (including approximately 1, 2, 5, 10, 20, 50, 60, 70, 80, 90, 100, 200, 500, and 1000 nm and all integers and fractional integers in between).

In some embodiments, the nanoparticle has at least one dimension, e.g., a diameter, of approximately 100 nm. In some embodiments, the nanoparticle has a diameter of approximately 200 nm. In other embodiments, the nanoparticle has a diameter of approximately 500 nm. In some embodiments, the nanoparticle has a diameter of approximately 1000 nm (1 μm). In such embodiments, the particle also can be referred to as a “microparticle”. Thus, the term “microparticle” includes particles having at least one dimension in the range of approximately one micrometer (μm), i.e., 1×10⁻⁶ meters, to approximately 1000 μm. The term “particle” as used herein is meant to include nanoparticles and microparticles.

It will be appreciated by one of ordinary skill in the art that nanoparticles suitable for use with the presently disclosed methods can exist in a variety of shapes, including but not limited to, spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. In some embodiments, a nanoparticle has a spherical shape.

As used herein, the term “associated with” refers to a first entity being linked by a direct or indirect covalent or non-covalent interaction to a second entity. Preferably, the association is covalent. Desirable non-covalent interactions include hydrogen bonding, van der Waals interactions, hydrophobic interactions, magnetic interactions, electrostatic interactions, and the like.

A used herein, the term “biocompatible” refers to compounds that are not toxic to cells. Compounds are “biocompatible” if their addition to cells in vitro results in less than or equal to 20% cell death and their administration in vivo does not induce inflammation or other such adverse effects.

As used herein, the term “biodegradable” refers to compounds that, when introduced into cells, are broken down by the cellular machinery or by hydrolysis into components that the cells either reuse or eliminate without significant toxic effect on the cells (i.e., fewer than approximately 20% of the cells are killed when the components are added to cells in vitro). The components preferably do not induce inflammation or other adverse effects in vivo. In some embodiments, the chemical reactions relied upon to break down the biodegradable compounds are uncatalyzed.

As used herein, the term “peptide” or “protein” refers to a string of at least three amino acids linked together by peptide bonds. The terms “protein” and “peptide” may be used interchangeably. Peptide may refer to an individual peptide or a collection of peptides. In some embodiments, peptides contain only natural amino acids although peptides may also contain non-natural amino acids (i.e., compounds that do not occur in nature but that can be incorporated into a polypeptide chain) and/or amino acid analogs known in the art. Also, one or more of the amino acids in a peptide may be modified, for example, by the addition of a chemical entity such as a carbohydrate group, a phosphate group, a farnesyl group, an isofarnesyl group, a fatty acid group, or a linker for conjugation, functionalization, or other modification, etc. In some embodiments, the modifications of the peptide provide a more stable peptide (e.g., having an improved half-life in vivo). These modifications may include cyclization of the peptide, the incorporation of D-amino acids into the peptide, etc. None of the modifications should substantially interfere with the desired biological activity of the peptide.

As used herein, the term “polynucleotide” or “oligonucleotide” refers to a polymer of nucleotides. Typically, a polynucleotide comprises at least three nucleotides. A polynucleotide may contain natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

### DESCRIPTION

Despite increased immunotherapy approvals in recent years, many solid tumors remain difficult to treat due to a lack of effective antigen presentation and T-cell activation within the tumor. Local expression of 4-1BBL and IL-12 can reprogram tumor cells into tAPCs and effectively drive anti-tumor response. However, clinical translation of this strategy faces scalability and regulatory challenges. To address these barriers, the technology provides bicistronic constructs that co-express both immune modulating genes from a single plasmid, thus simplifying delivery and reducing manufacturing costs. Further, the technology provides constructs that do not comprise an antibiotic resistance gene, thus improving therapeutic efficacy. These findings support eventual translation of the tAPC NPs into clinical trials.

IL-12 has been explored as a treatment for its anti-cancer effects because it activates T cells and natural killer (NK) cells and enhances the immune system's ability to recognize and eliminate tumor cells. IL-12 has been delivered both alone as a recombinant protein or as a gene therapy, and synergistically with chemotherapy, radiotherapy, and other immunotherapies (e.g., cancer vaccines and checkpoint inhibitors). However, despite its potency, IL-12 in any form has not been approved for cancer treatment due to dose-limiting toxicity associated with systemic delivery. Prior studies have demonstrated that confinement of IL-12 to the tumor microenvironment via an intratumoral injection may limit systemic toxicities. The technology provided herein connects IL-12 to 4-1BBL via a flexible (G4S)5 linker, which is commonly used in protein engineering and design. The advantages of physically tethering the IL-12 to 4-1BBL were twofold: 1) to reduce systemic toxicities resulting from secreted IL-12 exiting the tumor microenvironment after intratumoral injection; and 2) to potentially increase T-cell activation due to the spatial proximity of signal 2 and signal 3. Altering the length of the GS tether did not appear to influence IFNγ expression in vitro, although the longest linker length tested exhibited greater variance and slightly (though not statistically significant) lower levels of IFNγ. While a decrease in secreted IL-12 was observed, the tethered construct ultimately did not result in a greater anti-tumor response compared to the co-delivered 4-1BBL and IL-12 formulations in vivo or in vitro. However, there may be potential to employ a tethered 4-1BBL and IL-12 to strengthen T-cell responses in applications that utilize other strategies for local IL-12 administration.

Bicistronic plasmids that comprise 2A peptides express two distinct proteins in approximately equimolar ratios. Ribosome skipping produces most of the 2A domain remaining on the C-terminus of the protein that is expressed first, while a single proline scar remains on the N-terminus of the protein expressed second. Due to the short sequence of the 2A peptide, it has been reported that protein functions are not significantly impacted. However, experiments conducted during the development of embodiments of the technology described herein to characterize the expression of 4-1BBL and IL-12 indicated that expression of 4-1BBL following the T2A domain was significantly lower than when 4-1BBL was located before the T2A domain.

In this study, data demonstrated that bicistronic plasmids produced significantly improved survival durations compared to luciferase NPs and systemic IL-12 delivery without losing therapeutic efficacy compared to co-delivered 4-1BBL/IL-12 plasmids. Interestingly, while IFNγ levels were significantly higher in vitro for 4-1BBL and IL-12 delivered as separate plasmids compared to the 4T12 or IL-12 T2A 41-BBL, this difference was not observed in vivo. The median survival time in mice treated with 4T12 was the longest: 53 days following a dosing regimen of three injections. PBAEs offer the advantage of being both biodegradable and biocompatible, which allows multiple administrations without observed toxic or immunogenic side effects. Administering additional injections could potentially extend the survival time even further without toxicity. Moreover, tumor rechallenge demonstrated slower formation of a new tumor compared to previously untreated mice, reflecting the establishment of anti-tumor memory response in some animals. Together, these studies demonstrate that the bicistronic 4T12 plasmid is a viable construct for translation into the clinic.

Although 4-4-6 NPs were used for in vitro validation of plasmids, 5-3-6 NPs were used in vivo because this formulation was previously validated for delivery in mouse models. Characterization of the NPs demonstrated that all NPs were ~200 nm in diameter and displayed a negative zeta potential, thus indicating that the therapeutic effects from NP groups are not due to differences in biophysical properties of the NPs. Previous studies have indicated that the negative charge of the particles may also help with solid tumor delivery in vivo. The tumor extracellular matrix (ECM) is generally negatively charged, and delivery of a negatively charged nanoparticle may reduce nanoparticle interactions with the ECM, leading to deeper tumor penetration.

Another gene therapy safety concern relates to the risk of transferring antibiotic resistance genes to bacteria in the human microbiome. Antibiotic resistance represents a growing global health concern, making the use of antibiotic resistance genes an important regulatory consideration in gene therapies. Research-grade plasmids often contain an antibiotic resistance gene as a selection marker. However, in the context of the human microbiome, these plasmids may undergo horizontal gene transfer of their antibiotic resistance genes. As a result, the use of antibiotic resistance genes is discouraged by regulatory agencies. Thus, the technology described herein provides new plasmid vectors without antibiotic selection markers to maximize compliance with regulatory requirements. The Nanoplasmid vector from Aldevron was used to deliver 4-1BBL and IL-12 and Nanoplasmid was evaluated compared to the pUNO1 plasmids in vivo. Data indicated that the Nanoplasmids exhibit similar response rates compared to pUNO1 plasmids, and Nanoplasmids may even have a therapeutic advantage due to improved delivery efficiency. Future work entails testing bicistronic Nanoplasmid constructs encoding 4T12.

Embodiments of the technology provided herein align with current GMP and regulatory standards and lay the groundwork for eventual translation into clinical trials. Safe and effective non-viral reprogramming of the tumor immune microenvironment has the potential to induce endogenous cellular immune responses to treat both local solid tumors and systemic metastatic disease.

As noted above, prior technologies have been developed to deliver signal 2 (e.g., 4-1BBL) and signal 3 (e.g., IL-12) co-stimulatory molecules to cells (e.g., using nanoparticles) on multiple (i.e., more than one) genetic constructs and induce anti-tumor immune responses through T-cell activation.

The present technology improves upon current methods by expressing the signal 2 and signal 3 co-stimulatory molecules from a single genetic construct (a single plasmid), which facilitates translation of the present technology to clinical use.

In some embodiments, the technology provides engineered fusion proteins that comprise: 1) a signal 2 immune co-stimulatory protein that is expressed on the surface of a cell (e.g., in the cell membrane); 2) a signal 3 soluble molecule that stimulates immune cells (e.g., a cytokine); and 3) a linker or tether linking the signal 2 and signal 3 polypeptides.

In some embodiments, the technology also provides nucleic acids comprising a nucleotide sequence encoding engineered fusion proteins that comprise: 1) a signal 2 immune co-stimulatory protein that is expressed on the surface of a cell (e.g., in the cell membrane); 2) a signal 3 soluble molecule that stimulates immune cells (e.g., a cytokine); and 3) a linker or tether linking the signal 2 and signal 3 polypeptides. In some embodiments, nucleic acids comprise other elements that modulate the expression of the engineered fusion protein.

In some embodiments, the technology provides nucleic acids comprising a bicistronic gene that encodes a signal 2 immune co-stimulatory protein that is expressed on the surface of a cell (e.g., in the cell membrane); and a signal 3 soluble molecule that stimulates immune cells (e.g., a cytokine) within one nucleic acid. In some embodiments, the nucleic acids comprise nucleotide sequences that encode other signals in addition to at least one signal 2 polypeptide and one signal 3 polypeptide.

Signal 2 is one of the two signals that bind to T cells for activating T cells in humans. signal 2 is a secondary co-activation signal protein that binds with signal 1 (i.e., an antigen-loaded major histocompatibility complex (MHC) (e.g., MHC-I, MHC-II)/human leukocyte antigens (HLA)) to a T cell receptor (TCR) to activate the T cell. Exemplary signal 2 proteins include 4-1BBL, CD28, CD80, CD86, OX40L, and GITRL. In some embodiments, signal 2 is 4-1BBL.

Signal 3 is a cytokine that binds to an immune cell and causes amplification of a T cell response. In some embodiments, signal 3 directs and amplifies T cell differentiation and expansion. Exemplary signal 3 proteins include interleukins and cytokines, such as the transforming growth factor (TGF) beta family of cytokines, including TGF-ß1, TGF-ß2, TGF-ß3, and TGF-ß4. Exemplary interleukins include, but are not limited to, IL-1, IL-2, IL; 3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In some embodiments, the signal 3 protein is selected from the group consisting of IL-6, IL-7, IL-15, IL-18, IL-21, IFN-α, and IFN-ß. In some embodiments, the signal 3 protein is IL-2 or IL-12. In some embodiments, signal 3 is IL-12.

The technology described herein finds use as a therapeutic for treating human diseases, e.g., any diseases that has an immune system component such as cancer, infectious diseases, and autoimmune diseases.

### Compositions

The technology provided herein relates to compositions comprising engineered proteins comprising a signal 2 immune co-stimulatory molecule expressed on the surface of a cell, a signal 3 soluble molecule that stimulates immune cells such as a cytokine, and a linker or tether between these two molecules. In some embodiments, the linker comprises an amino acid sequence that causes ribosome skipping during translation and thus produces two separate polypeptides from a single open reading frame. In some embodiments, the linker is a 2A peptide, e.g., T2A, P2A, E2A, or F2A (SEQ ID NO: 1-4). In some embodiments, the linker is a 2A peptide comprising the amino acid sequence GSG at the N-terminus to improve efficiency of ribosomal skipping. Thus, in some embodiments, the linker is an improved T2A, P2A, E2A, or F2A peptide as provided by SEQ ID NO: 6-9. See, e.g., Liu (2017) “Systematic comparison of 2A peptides for cloning multi-genes in a polycistronic vector”. Scientific Reports 7:2193; Karuna and Roy (eds) Live Imaging In Zebrafish: Insights Into Development And Disease (World Scientific, 2010), pages 51-52; Luke (2008) “Occurrence, function and evolutionary origins of ‘2A-like’ sequences in virus genomes” The Journal of General Virology 89:1036-42; Yang (2017) “Structures and Corresponding Functions of Five Types of Picornaviral 2A Proteins” Frontiers in Microbiology 8:1373; and Ryan (1991) “Cleavage of foot-and-mouth disease virus polyprotein is mediated by residues located within a 19 amino acid sequence” The Journal of General Virology 72:2727-32, each of which is incorporated herein by reference. In some embodiments, the polypeptide comprises signal 2, the linker, and signal 3 in order from N-terminus to C-terminus. In some embodiments, the polypeptide comprises signal 3, the linker, and signal 2 in order from N-terminus to C-terminus.

In some embodiments, the polypeptide comprises a tether sequence that tethers the signal 2 polypeptide and the signal 3 polypeptide. In some embodiments, the tether sequence comprises an amino acid sequence comprising a repeated sequence GGGGS (SEQ ID NO: 14). In some embodiments, the tether sequence comprises an amino acid sequence comprising (GGGGS)n (SEQ ID NO: 14), wherein n=1, 2 3, 4, 5, 6, 7, 8, 9, or 10. In some embodiments, tether sequence comprises the sequence GGGGS GGGGS GGGGS GGGGS GGGGS (SEQ ID NO: 10). In some embodiments, the polypeptide comprises signal 2, the tether, and signal 3 in order from N-terminus to C-terminus. In some embodiments, the polypeptide comprises signal 3, the tether, and signal 2 in order from N-terminus to C-terminus.

In some embodiments, the technology provided herein relates to a therapeutic agent comprising a single plasmid that reprograms cells (e.g., cancer cells) to activate an immune response to attack themselves and other cancer cells. As described below, the single plasmid comprises a nucleotide sequence that encodes a signal 2 polypeptide and a nucleotide sequence that encodes a signal 3 polypeptide.

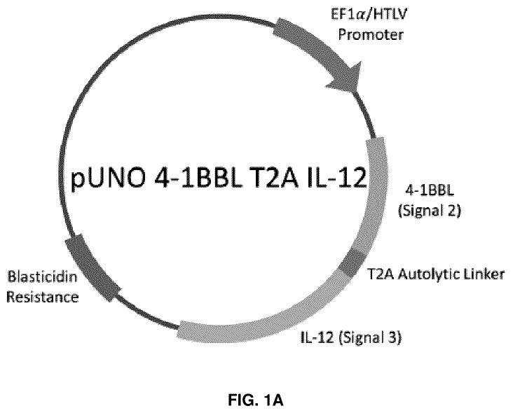

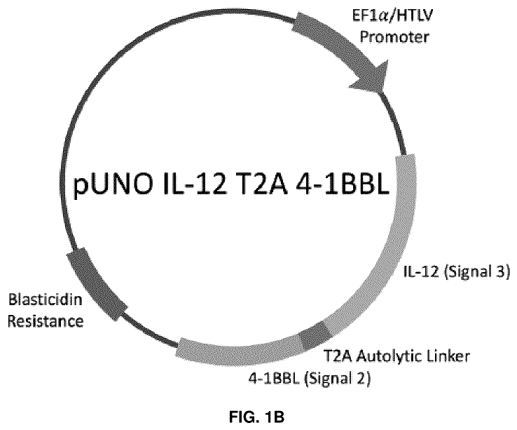

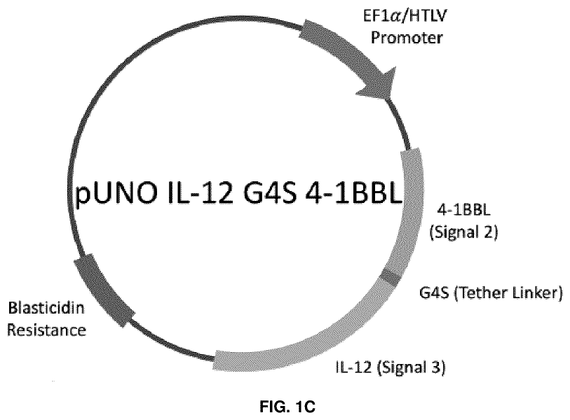

In some embodiments, the single plasmid comprises a nucleotide sequence that encodes a linker amino acid sequence that links the signal 2 polypeptide and the signal 3 polypeptide. See, e.g., FIG. 1A and FIG. 1B. In some embodiments, the linker comprises an amino acid sequence that causes ribosome skipping during translation and thus produces two separate polypeptides from a single open reading frame. In some embodiments, the linker is a 2A peptide, e.g., T2A, P2A, E2A, or F2A (SEQ ID NO: 1-4). In some embodiments, the linker is a 2A peptide comprising the amino acid sequence GSG at the N-terminus to improve efficiency of ribosomal skipping. Thus, in some embodiments, the linker is an improved T2A, P2A, E2A, or F2A peptide as provided by SEQ ID NO: 6-9. See, e.g., Liu (2017) “Systematic comparison of 2A peptides for cloning multi-genes in a polycistronic vector”. Scientific Reports 7: 2193; Karuna and Roy (eds) Live Imaging In Zebrafish: Insights Into Development And Disease (World Scientific, 2010), pages 51-52; Luke (2008) “Occurrence, function and evolutionary origins of ‘2A-like’ sequences in virus genomes” The Journal of General Virology 89: 1036-42; Yang (2017) “Structures and Corresponding Functions of Five Types of Picornaviral 2A Proteins” Frontiers in Microbiology 8: 1373; and Ryan (1991) “Cleavage of foot-and-mouth disease virus polyprotein is mediated by residues located within a 19 amino acid sequence” The Journal of General Virology 72: 2727-32, each of which is incorporated herein by reference. In some embodiments, the single plasmid comprises a nucleotide sequence encoding signal 2, the linker, and signal 3 in order from 5′ to 3′. In some embodiments, the single plasmid comprises a nucleotide sequence encoding signal 3, the linker, and signal 2 in order from 5′ to 3′. See, e.g., FIG. 1A and FIG. 1B.

In some embodiments, the single plasmid may comprise a nucleotide sequence that encodes a tether amino acid sequence that tethers the signal 2 polypeptide and the signal 3 polypeptide. In some embodiments, the tether sequence comprises an amino acid sequence comprising a repeated sequence GGGGS (SEQ ID NO: 14). In some embodiments, the tether sequence comprises an amino acid sequence comprising (GGGGS)n (SEQ ID NO: 14), wherein n=1, 2 3, 4, 5, 6, 7, 8, 9, or 10. In some embodiments, tether sequence comprises the sequence GGGGS GGGGS GGGGS GGGGS GGGGS (SEQ ID NO: 10). See, e.g., FIG. 1C. In some embodiments, the single plasmid comprises a nucleotide sequence encoding signal 2, the tether, and signal 3 in order from 5′ to 3′. See, e.g., FIG. 1C. In some embodiments, the single plasmid comprises a nucleotide sequence encoding signal 3, the tether, and signal 2 in order from 5′ to 3′.

Alternatively, the single plasmid may comprise a nucleotide sequence that is a bicistronic gene encoding the signal 2 polypeptide and the signal 3 polypeptide. That is, the single plasmid may comprise a nucleotide sequence that is a bicistronic gene encoding the signal 2 polypeptide and the signal 3 polypeptide and does not comprise a nucleotide sequence that encodes a linker that links the signal 2 polypeptide and the signal 3 polypeptide and does not comprise a nucleotide sequence that encodes a tether that tethers the signal 2 polypeptide and the signal 3 polypeptide (a “tether-free construct”).

In some embodiments, the therapeutic agent (i.e., a single plasmid comprising a nucleotide sequence that encodes a signal 2 polypeptide and a nucleotide sequence that encodes a signal 3 polypeptide) is provided as a gene delivery nanoparticle formulation comprising polymers and the single plasmid. The gene delivery nanoparticle formulation finds use in reprogramming cancer cells to activate an immune response to attack themselves and other cancer cells. The presently disclosed formulations find use in killing cancer cells and/or to reduce tumor size in various types of cancer.

For example, embodiments of the presently disclosed subject matter uses synthetic, biodegradable nanoparticles (NPs) to reprogram tumor cells into “tumor-derived APCs” (referred to herein as “tAPCs”) in vivo to activate T cells and natural killer (NK) cells for systemic tumor rejection. Antigen-presenting cells (APCs) activate T-cells by presenting coordinated signals, including antigen (signal 1); surface-bound co-stimulatory molecules (signal 2); and secreted cytokines (signal 3). Many tumor cells express signal 1 (tumor antigen associated with MHC I. Delivering signal 2 and signal 3 using the present technology increases signal 1 expression, further improving the immunogenicity of the tAPCs. Accordingly, the technology described herein provides an antigen agnostic therapy that elicits a systemic immune response targeting multiple antigens expressed by tumor cells. Moreover, the technology descried herein presents activating signals to NK cells, which often have been implicated in tumor control in cases of successful immunotherapy.

Previous research has indicated that the soluble signal 3 recruits cells and affects their cell fate, and signal 2 expression on cancer cells causes activation of immune cells directly against cancer. Thus, colocalized presentation of signal 2 and signal 3 with signal 1 provides a robust anti-cancer therapy. Accordingly, the technology described herein provides an approach for colocalized expression of signal 2 and signal 3 on signal 1-bearing tumor cells. As a result, T cells are directly activated in the context of the tumor antigen, which provides an antigen-specific cellular response. The colocalized expression of these immune-stimulatory molecules minimizes and/or eliminates unacceptable levels of adverse side effects associated with other therapies comprising systemic delivery of cytokines and signal 2 agonists.

Accordingly, the presently disclosed subject matter provides formulations (e.g., nanoparticle formulations) and methods of their use for inducing tumor cells to express co-stimulatory molecules and cytokines for T-cell and NK cell activation. In some embodiments, the presently disclosed subject matter provides a composition comprising a single plasmid comprising: 1) a genetic element that encodes a signal 2 (e.g., a cell surface bound protein that regulates immune cells, such as 4-1BBL, CD80, CD86, and OX40L); and 2) a genetic element that encodes a signal 3 (e.g., a secreted protein that regulates immune cells, such as IL-2, IL-12, IL-6, IL-7, IL-15, IL-18, IL-21, IFN-α, and IFN-b). In some embodiments, the single plasmid further comprises a linker or tether sequence.

In some embodiments, the present technology provides a composition comprising: 1) a single plasmid comprising a genetic element that encodes a signal 2 (e.g., a cell surface bound protein that regulates immune cells, such as 4-1BBL, CD80, CD86, and OX40L) and a genetic element that encodes a signal 3 (e.g., a secreted protein that regulates immune cells, such as IL-2, IL-12, IL-6, IL-7, IL-15, IL-18, IL-21, IFN-α, and IFN-b); and 2) a cationic biomaterial or biomaterial blend that encapsulates the single plasmid into a nanoparticle. In some embodiments, the single plasmid further comprises a linker or tether sequence. In some embodiments, the cationic biomaterial or biomaterial blend comprises a cationic polymer. In some embodiments, the cationic biomaterial or biomaterial blend comprises a cationic biodegradable polymer or a number of cationic biodegradable polymers. In some embodiments, the cationic biodegradable polymer is poly(lactic-co-glycolic acid) (PLGA), polycaprolactone (PCL), polyglycolide (PGA), poly(lactic acid) (PLA), polyhydroxyalkanoate (PHA) (e.g., poly-3-hydroxybutyrate (P3HB), poly(acrylic acid) (PAA), poly(3-hydroxybutyrate-co-3-hydroxyvalerate) (PHBV)), poly(beta-amino ester) (PBAE), or combinations thereof, or other hydrolytically biodegradable polymers. See, e.g., Int'l Pat. App. Pub. No. WO 2020/198145, which is incorporated herein by reference. As used herein, “biodegradable” polymers and/or nanoparticles are those that, when introduced into cells, are broken down by the cellular machinery or by hydrolysis into components that the cells can either reuse or dispose of without significant toxic effect on the cells (i.e., fewer than approximately 20% of the cells are killed when the components are added to cells in vitro). Such components preferably do not induce inflammation or other adverse effects in vivo. In certain preferred embodiments, the chemical reactions relied upon to break down the biodegradable compounds are uncatalyzed. In some embodiments, the biodegradable nanoparticles comprise a chemical moiety having one or more degradable linkages, such as an ester linkage, a disulfide linkage, an amide linkage, an anhydride linkage, and a linkage susceptible to enzymatic degradation. In some embodiments, the cationic biomaterial or biomaterial blend comprises a PBAE, e.g., as described in U.S. Pat. Nos. 9,884,118; 9,802,984; 9,717,694; 8,992,991; and 8,287,849; and in U.S. Pat. App. Pub. Nos. 20180256745; 20180112038; 20170216363; 20150273071, each of which is incorporated herein by reference.

In some embodiments, compositions comprise a nanoparticle comprising a polymer complexed with a single plasmid comprising a nucleotide sequence that encodes a signal 2 polypeptide and a nucleotide sequence that encodes a signal 3 polypeptide. In some embodiments, the single plasmid further comprises a linker or tether sequence. In some embodiments, the nanoparticle finds use in transfecting cancer cells in vivo and/or in vitro.

In embodiments, the nanoparticles are spherical in shape. In embodiments, the particles have a non-spherical shape. In embodiments, the particles have an ellipsoidal shape with an aspect ratio of the long axis to the short axis between 2 and 10. Spherical particles have a size characterized by a measurement of a diameter. Non-spherical particles have a size that may be characterized by a measurement in at least one dimension.

In some embodiments, the nanoparticle has a size (e.g., a diameter and/or at least one dimension) in the range of 50 to 500 nm. In embodiments, the nanoparticle has at least one dimension in the range of approximately 50 nm to approximately 500 nm or from approximately 50 to approximately 200 nm. Exemplary particles may have an average size (e.g., average diameter and/or at least one dimension) of approximately 50, approximately 75, approximately 100, approximately 125, approximately 150, approximately 200, approximately 250, approximately 300, approximately 400 or approximately 500 nm. In some embodiments, the nanoparticle has an average diameter and/or at least one dimension of from approximately 50 nm to approximately 500 nm, from approximately 50 nm to approximately 300 nm, from approximately 50 nm to approximately 200 nm, from approximately 50 nm to approximately 150 nm, or from approximately 70 to 100 nm. In embodiments, the nanoparticle has an average diameter and/or at least one dimension of from approximately 200 nm to approximately 500 nm. In embodiments, the nanoparticle has at least one dimension, e.g., average diameter, of approximately 50 to approximately 100 nm. Nanoparticles are usually desirable for in vivo applications. For example, a nanoparticle of less than approximately 200 nm will better distribute to target tissues in vivo.

In some embodiments, nanoparticles that comprise a single plasmid as described herein are further encapsulated into a larger nanoparticle, microparticle, or device. In some embodiments, the larger nanoparticle, microparticle, or device is degradable and in some embodiments the larger nanoparticle, microparticle, or device is not degradable and instead provides a reservoir that can be refilled with the nanoparticles. The larger nanoparticles, microparticles, and/or devices can be constructed with any biomaterials and methods known by one of ordinary skill in the art. In some embodiments, the larger nanoparticle, microparticle, or device is constructed with multi-component degradable cationic polymers as described herein.

In some embodiments, a nanoparticle is targeted to a particular cell type using through biomaterial selection, nanoparticle biophysical properties, and/or a targeting ligand). In some embodiments, a nanoparticle is targeted to a particular cell type using transcriptional targeting of a therapeutic gene to a particular cell type (e.g., cancer cells). Transcriptional targeting includes designing a nucleic acid cargo (e.g., a single plasmid as described herein) to comprise a promoter that is active in cells or tissue types of interest so that the delivered nanoparticles express the nucleic acid cargo in a tissue-specific manner.

### Methods

The technology provided herein relates to methods for treating a cancer. For example, in some embodiments, methods comprise administering a composition or formulation comprising the therapeutic agent described herein (i.e., a single plasmid comprising a nucleotide sequence that encodes a signal 2 polypeptide and a nucleotide sequence that encodes a signal 3 polypeptide) to a subject in need of a cancer treatment. In some embodiments, methods comprise administering a composition or formulation comprising the therapeutic agent described herein (i.e., a single plasmid comprising a nucleotide sequence that encodes a signal 2 polypeptide and a nucleotide sequence that encodes a signal 3 polypeptide) provided as a gene delivery nanoparticle formulation comprising polymers and the single plasmid.

More generally, the technology described herein provides a method for reprogramming one or more cancer cells into one or more tumor-derived antigen-presenting cells (tAPCs), wherein the one or more tAPCs mimic a natural antigen presenting cell (APC) and direct an immune response against themselves and other cancer cells. For example, embodiments of methods comprise transfecting one or more cancer cells with the therapeutic agent described herein (i.e., a single plasmid comprising a nucleotide sequence that encodes a signal 2 polypeptide and a nucleotide sequence that encodes a signal 3 polypeptide). In some embodiments, transfecting one or more cancer cells with the therapeutic agent described herein (i.e., a single plasmid comprising a nucleotide sequence that encodes a signal 2 polypeptide and a nucleotide sequence that encodes a signal 3 polypeptide) comprises contacting a cancer cell with the therapeutic agent described herein (i.e., a single plasmid comprising a nucleotide sequence that encodes a signal 2 polypeptide and a nucleotide sequence that encodes a signal 3 polypeptide) provided as a gene delivery nanoparticle formulation comprising polymers and the single plasmid.

In some embodiments, transfecting one or more cancer cells promotes an immune cell activation against one or more antigens expressed on one or more cancer cells. In some embodiments, the one or more tAPCs activate an antigen specific T-cell response against MHC 1+ tumor cells. In other embodiments, the one or more tAPCs provide an activating signal to one or more natural killer (NK) cells to induce anti-tumor cytotoxicity therein. In certain embodiments, the one or more tAPCs activate an antigen-independent NK cell response against MHC I-/low tumor cells.

Accordingly, the methods described herein induce a systemic immune response resulting in cell death of distant metastases. In particular, methods for treating a cancer comprise transfecting one or more cancer cells in a subject in need of treatment with a composition disclosed herein.

Any cancer may be treated using the methods described herein. A “cancer” in a subject refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within a subject, or circulate in the blood stream as independent cells, for example, leukemic cells.

A cancer can include, but is not limited to, acute lymphocytic leukemia, acute myelogenous leukemia, angiosarcoma, basal cell carcinoma, bladder cancer, brain cancer (e.g., gliomas), breast cancer, cervical cancer, choriocarcinoma, colon cancer, colorectal cancer, corpus uteri cancer, endocrine cancer, esophageal cancer, Ewing's Sarcoma, eye or ocular cancer, gastrointestinal cancer, head cancer, head and neck cancer, hemangioendothelioma, hemangiomas, hepatocellular carcinoma (HCC), Kaposi's Sarcoma, larynx cancer, leukemia/lymphoma, liver cancer, lung cancer, lymphoma, lymphangiogenesis, melanoma, mouth/pharynx cancer, neck cancer, neuroblastoma, neurofibromatosis, oral cancer, ovarian cancer, pancreatic cancer, prostate cancer, rectal cancer, renal cancer, rhabdomyosarcoma, stomach cancer, skin cancer, small cell lung cancer, squamous cell carcinoma, testicular cancer, throat cancer, tuberous sclerosis, urinary cancer, uterine cancer, Wilms Tumor, benign and malignant tumors, and adenomas.

In some embodiments, the cancer is selected from the group consisting of a melanoma, a breast cancer (e.g., triple-negative breast cancer), colorectal cancer, liver cancer, and brain cancer (e.g., a glioma).

In some embodiments, methods comprise administering to the subject one or more therapeutic agents simultaneously or sequentially with a composition or formulation comprising the therapeutic agent described herein (i.e., a single plasmid comprising a nucleotide sequence that encodes a signal 2 polypeptide and a nucleotide sequence that encodes a signal 3 polypeptide).

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit” and grammatical derivations thereof refer to the ability of a composition or formulation comprising the therapeutic agent described herein (i.e., a single plasmid comprising a nucleotide sequence that encodes a signal 2 polypeptide and a nucleotide sequence that encodes a signal 3 polypeptide) to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of a therapeutic agent or drug delivery device refers to the amount necessary to elicit the desired biological response. As will be appreciated by those of ordinary skill in this art, the effective amount of a therapeutic agent or drug delivery device may vary depending on such factors as the desired biological endpoint, the agent to be delivered, the makeup of the pharmaceutical composition, the target tissue, and the like.

As used herein, the term “combination” is used in its broadest sense and means that a subject is administered at least two agents, e.g., a composition or formulation comprising the therapeutic agent described herein (i.e., a single plasmid comprising a nucleotide sequence that encodes a signal 2 polypeptide and a nucleotide sequence that encodes a signal 3 polypeptide) and at least one second therapeutic agent. More particularly, the term “in combination” refers to the concomitant administration of two (or more) active agents for the treatment of, e.g., a disease state. As used herein, the active agents may be combined and administered in a single dosage form, may be administered as separate dosage forms at the same time, or may be administered as separate dosage forms that are administered alternately or sequentially on the same or separate days. In some embodiments, the active agents are combined and administered in a single dosage form. In some embodiments, the active agents are administered in separate dosage forms (e.g., wherein it is desirable to vary the amount of one but not the other). The single dosage form may include additional active agents for the treatment of the disease state.

Further, the composition or formulation comprising the therapeutic agent described herein (i.e., a single plasmid comprising a nucleotide sequence that encodes a signal 2 polypeptide and a nucleotide sequence that encodes a signal 3 polypeptide) can be administered alone or in combination with adjuvants that enhance the stability of the composition, formulation, and/or therapeutic agent or that facilitate administration of pharmaceutical compositions containing the composition, formulation, and/or therapeutic agent. In some embodiments, the composition or formulation comprising the therapeutic agent described herein (i.e., a single plasmid comprising a nucleotide sequence that encodes a signal 2 polypeptide and a nucleotide sequence that encodes a signal 3 polypeptide) can be administered alone or in combination with adjuvants that provide increased dissolution or dispersion, increase inhibitory activity, or provide adjunct therapy. Advantageously, in some embodiments, combination therapies comprise lower dosages of the conventional therapeutics than the dosages used for the conventional therapeutic alone, thus avoiding possible toxicity and adverse side effects incurred when the conventional therapeutic agents are used alone as monotherapies.

The timing of administration of the composition or formulation comprising the therapeutic agent described herein (i.e., a single plasmid comprising a nucleotide sequence that encodes a signal 2 polypeptide and a nucleotide sequence that encodes a signal 3 polypeptide) and at least one additional therapeutic agent can be varied so long as the beneficial effects of the combination of these agents are achieved. Accordingly, the phrase “in combination with” refers to the administration of composition or formulation comprising the therapeutic agent described herein and at least one additional therapeutic agent either simultaneously, sequentially, or a combination thereof.

Therefore, a subject administered a combination of composition or formulation comprising the therapeutic agent described herein and at least one additional therapeutic agent can receive a composition or formulation comprising the therapeutic agent described herein and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, or 240 minutes or longer of one another. In some embodiments, agents are administered sequentially and are administered within 1, 5, 10, 15, 20, or more days of one another. Where the composition or formulation comprising the therapeutic agent described herein and at least one additional therapeutic agent are administered simultaneously, they can be administered to the subject as separate pharmaceutical compositions, each comprising either a composition or formulation comprising the therapeutic agent described herein or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy”, “synergistic”, “synergistically”, and derivations thereof, such as in a “synergistic effect” or a “synergistic combination” or a “synergistic composition”, refer to circumstances under which the biological activity of a combination of a composition or formulation comprising the therapeutic agent described herein and at least one additional therapeutic agent is greater than the sum of the biological activities of the respective agents when administered individually.

In some embodiments, the presently disclosed composition or formulation comprising the therapeutic agent described herein (e.g., a single plasmid comprising a nucleotide sequence that encodes a signal 2 polypeptide and a nucleotide sequence that encodes a signal 3 polypeptide) is provided as a gene delivery nanoparticle formulation comprising polymers and the single plasmid and is administered in a variety of forms depending on the desired route and/or dose. For example, the nanoparticle gene delivery nanoparticle formulation can be administered in a pharmaceutically acceptable carrier. As used herein, “pharmaceutically acceptable carrier” is intended to include, but is not limited to, water, saline, dextrose solutions, human serum albumin, liposomes, hydrogels, microparticles, and nanoparticles.

Depending on the specific conditions being treated, the presently disclosed composition or formulation may be formulated into liquid or solid dosage forms and administered systemically or locally. The agents may be delivered, for example, in a timed-release or sustained-low release form as is known to those skilled in the art. Techniques for formulation and administration may be found in Remington: The Science and Practice of Pharmacy (20th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra articular, intra-sternal, intra-synovial, intra hepatic, intralesional, intracranial, intraperitoneal, intranasal, or intraocular injections or other modes of delivery.

While the form and/or route of administration can vary, in some embodiments the presently disclosed composition or formulation is administered parenterally (e.g., by subcutaneous, intravenous, or intramuscular administration) or in some embodiments is administered directly to the lungs. Local administration to the lungs can be achieved using a variety of formulation strategies including pharmaceutical aerosols, which may be solution aerosols or powder aerosols. Powder formulations typically comprise small particles. Suitable particles can be prepared using any means known in the art, for example, by grinding in an airjet mill, ball mill or vibrator mill, sieving, microprecipitation, spray-drying, lyophilization, or controlled crystallization. Typically, particles will be about 10 micrometers or less in diameter. Powder formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol, or sorbitol. Alternatively, solution aerosols may be prepared using any means known to those of skill in the art, for example, an aerosol vial provided with a valve adapted to deliver a metered dose of the composition. Where the inhalable form of the active ingredient is a nebulizable aqueous, organic, or aqueous/organic dispersion, the inhalation device may be a nebulizer, for example a conventional pneumatic nebulizer such as an airjet nebulizer, or an ultrasonic nebulizer, which may contain, for example, from 1 to 50 mL, commonly 1 to 10 mL, of the dispersion; or a hand-held nebulizer which allows smaller nebulized volumes, e.g. 10 μL to 100 μL. For injection, composition or formulation may be formulated and diluted in aqueous solutions, such as in physiologically compatible buffers such as Hank's solution, Ringer's solution, or physiological saline buffer.

Use of pharmaceutically acceptable inert carriers to formulate the compounds herein disclosed for the practice of the methods into dosages suitable for systemic administration is within the scope of the disclosure. With proper choice of carrier and suitable manufacturing practice, the compositions described herein, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the composition or formulation also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

In some embodiments, the technology provides a method of using and storing the polymers and particles described herein whereby a cryoprotectant (including, but not limited to, a sugar) is added to the polymer and/or particle solution and it is lyophilized and stored as a powder. Such a powder is designed to remain stable and be reconstituted easily with aqueous buffer as one skilled in the art could utilize.

### Kits

In some embodiments, the technology described herein provides a kit. For example, in some embodiments, kits comprise some or all of the components, reagents, supplies, and the like to practice a method as described herein. In some embodiments, the term “kit” refers to any intended article of manufacture (e.g., a package or a container) comprising a composition or formulation comprising the therapeutic agent described herein (i.e., a single plasmid comprising a nucleotide sequence that encodes a signal 2 polypeptide and a nucleotide sequence that encodes a signal 3 polypeptide) or a composition or formulation comprising the therapeutic agent described herein (i.e., a single plasmid comprising a nucleotide sequence that encodes a signal 2 polypeptide and a nucleotide sequence that encodes a signal 3 polypeptide) provided as a gene delivery nanoparticle formulation comprising polymers and the single plasmid.

In some embodiments, the kit is packaged in a divided or undivided container, such as a carton, bottle, ampule, tube, and the like. The presently disclosed composition or formulation can be packaged in dried, lyophilized, or liquid form. Additional components provided can include vehicles for reconstitution of dried components. Preferably, all such vehicles are sterile and apyrogenic so that they are suitable for injection into a patient without causing adverse reactions. In some embodiments, a kit further comprises one or more of multiple dosage units of the composition, a pharmaceutically acceptable carrier, a device for administration of the composition, instructions for use, and combinations thereof.

Although the disclosure herein refers to certain illustrated embodiments, it is to be understood that these embodiments are presented by way of example and not by way of limitation.

### EXAMPLES

Delivery of 4-1BB ligand (4-1BBL) and interleukin-12 (IL-12) via poly(8-amino ester) nanoparticles (PBAE-NPs) reprograms tumor cells into tumor-associated antigen-presenting cells (tAPCs), thus stimulating anti-tumor immune responses. The technology described herein provides an approach to address scalability and regulatory challenges for translating tAPC-delivering nanoparticles into the clinic by: (1) engineering bicistronic plasmids co-expressing 4-1BBL and IL-12 via a T2A peptide or a (G4S) 5 linker; and (2) utilizing antibiotic resistance-free Nanoplasmid backbones. During development of embodiments of this technology, experiments were conducted and data were collected that demonstrated bicistronic and Nanoplasmid-based tAPC NPs exhibited similar or improved survival compared to separate plasmid delivery of 4-1BBL and IL-12 in a B16-F10 tumor model. These results support the translation of bicistronic, antibiotic resistance gene-free plasmid strategies to advance tAPC PBAE-NPs and, more broadly, gene-delivered therapies toward the clinic. Accordingly, during the development of embodiments of the technology described herein, experiments were conducted to produce nucleic acid constructs expressing signal 2 and signal 3, test expression of signal 2 and signal 3 in melanoma cells, and test induction of an immune response in splenocytes.

### Materials and Methods

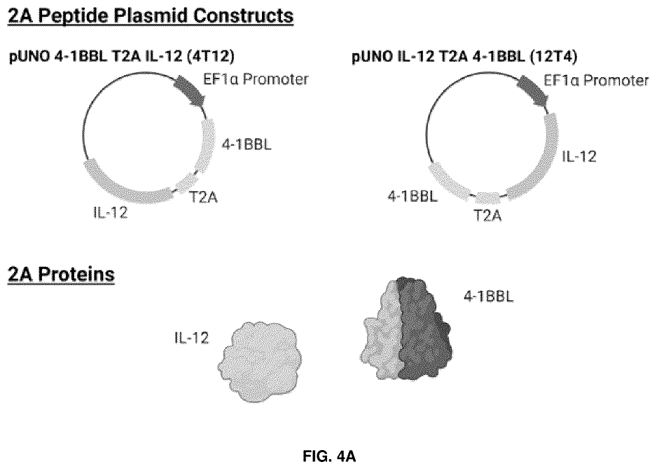

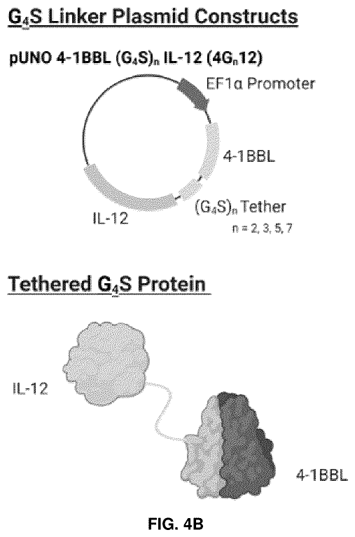

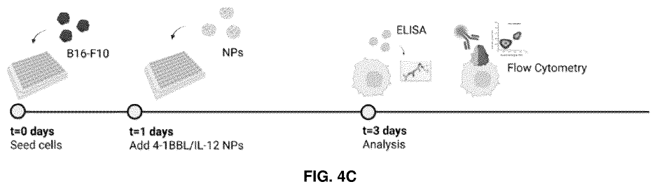

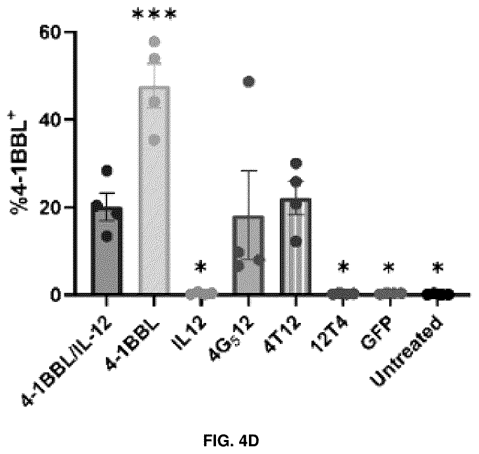

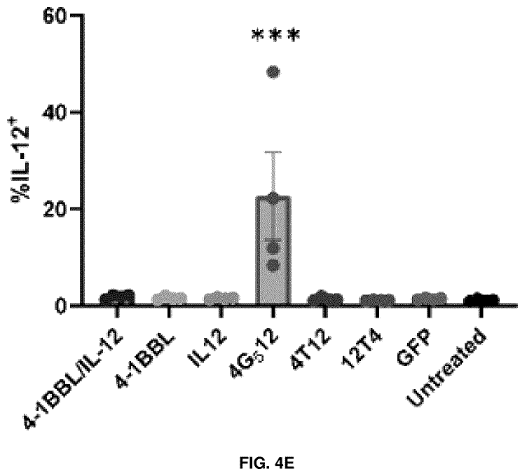

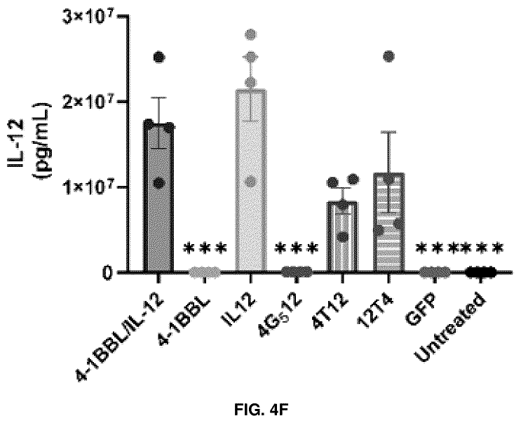

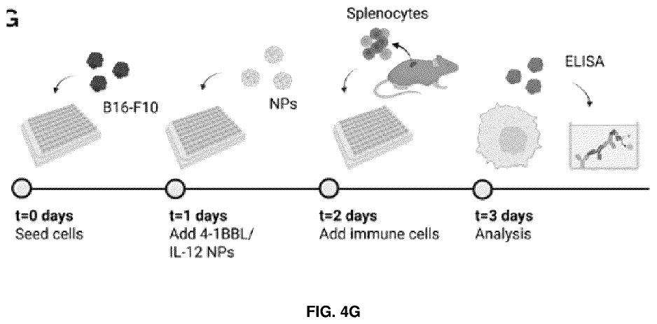

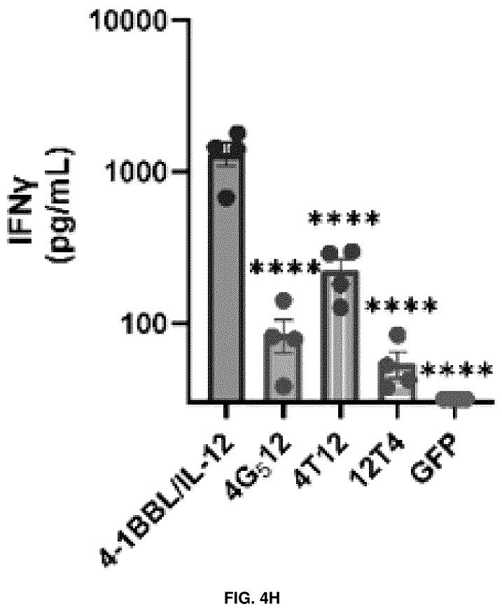

Plasmid Design—During the development of embodiments of the technology provided herein, plasmids were constructed using the pUNO1 backbone containing a Blasticydin antibiotic resistance gene, following standard cloning methods. Restriction enzymes NheI (NEB (New England Biolabs), catalog no. R3131S) and SalI (NEB, catalog no. R0138S) were used to remove the inserts from Clonal Genes (Twist Biosciences) encoding mouse 4-1BBL (residues 1-309 in 4G512, 4T12 and residues 566-875 in 12T4) and mouse IL-12 (P40: P35) (residues 335-851 in 4G512, residues 342-880, and residues 1-538 in 12T4) with the linkers (residues 310-334) and T2A sequences (residues 310-333 in 4T12 and 539-559 in 12T4) noted in FIG. 1 and FIG. 4A. Table 1 provides the coding region for all plasmids. In tandem, the pUNO1 backbone from the plasmid pUNO1 MCS (InvivoGen, catalog no. puno1-mcs) was digested with NheI and SalI. Ligation was performed using T4 DNA Ligase (NEB, M0202S). Sequences of plasmids were verified via Sanger sequencing.

TABLE 1
Encoded amino acid sequences
for 4-1BBL and IL-12 delivery

| | | SEQ |
|---|---|---|
| | IL-12 4-1BBL T2A GS Linker | ID NO: |
| 4G₅12 | MDQHTLDVEDTADARHPAGTSCPSDAALLRDTGLLADAALLSD | 11 |
| | TVRPTNAALPTDAAYPAVNVRDREAAWPPALNFCSRHPKLYGL | |
| | VALVLLLLIAACVPIFTRTEPRPALTITTSPNLGTRENNADQV | |
| | TPVSHIGCPNTTQQGSPVFAKLLAKNQASLCNTTLNWHSQDGA | |
| | GSSYLSQGLRYEEDKKELVVDSPGLYYVFLELKLSPTFTNTGH | |
| | KVQGWVSLVLQAKPQVDDFDNLALTVELFPCSMENKLVDRSWS | |
| | QLLLLKAGHRLSVGLRAYLHGAQDAYRDWELSYPNTTSFGLFL | |
| | VKPDNPWEGGGGSGGGGSGGGGSGGGGSGGGGSMWELEKDVYV | |
| | VEVDWTPDAPGETVNLTCDTPEEDDITWTSDQRHGVIGSGKTL | |
| | TITVKEFLDAGQYTCHKGGETLSHSHLLLHKKENGIWSTEILK | |
| | NFKNKTFLKCEAPNYSGRFTCSWLVQRNMDLKFNIKSSSSSPD | |
| | SRAVTCGMASLSAEKVTLDORDYEKYSVSCQEDVTCPTAEETL | |
| | PIELALEARQQNKYENYSTSFFIRDIIKPDPPKNLQMKPLKNS | |
| | QVEVSWEYPDSWSTPHSYFSLKFFVRIQRKKEKMKETEEGCNQ | |
| | KGAFLVEKTSTEVQCKGGNVCVQAQDRYYNSSCSKWACVPCRV | |
| | RSVPGVGVPGVGRVIPVSGPARCLSQSRNLLKTTDDMVKTARE | |
| | KLKHYSCTAEDIDHEDITRDQTSTLKTCLPLELHKNESCLATR | |
| | ETSSTTRGSCLPPQKTSLMMTLCLGSIYEDLKMYQTEFQAINA | |
| | ALQNHNHQQIILDKGMLVAIDELMQSLNHNGETLRQKPPVGEA | |
| | DPYRVKMKLCILLHAFSTRVVTINRVMGYLSSA | |
| 4T12 | MDQHTLDVEDTADARHPAGTSCPSDAALLRDTGLLADAALLSD | 12 |
| | TVRPTNAALPTDAAYPAVNVRDREAAWPPALNFCSRHPKLYGL | |
| | VALVLLLLIAACVPIFTRTEPRPALTITTSPNLGTRENNADQV | |
| | TPVSHIGCPNTTQQGSPVFAKLLAKNQASLCNTTLNWHSQDGA | |
| | GSSYLSQGLRYEEDKKELVVDSPGLYYVFLELKLSPTFTNTGH | |
| | KVQGWVSLVLQAKPQVDDFDNLALTVELFPCSMENKLVDRSWS | |
| | QLLLLKAGHRLSVGLRAYLHGAQDAYRDWELSYPNTTSFGLFL | |
| | VKPDNPWEELSGSGEGRGSLLTCGDVEENPGPLRSPGRVNMCP | |
| | QKLTISWFAIVLLVSPLMAMWELEKDVYVVEVDWTPDAPGETV | |
| | NLTCDTPEEDDITWTSDQRHGVIGSGKTLTITVKEFLDAGQYT | |
| | CHKGGETLSHSHLLLHKKENGIWSTEILKNFKNKTFLKCEAPN | |
| | YSGRFTCSWLVQRNMDLKFNIKSSSSSPDSRAVTCGMASLSAE | |
| | KVTLDQRDYEKYSVSCQEDVTCPTAEETLPIELALEARQQNKY | |
| | ENYSTSFFIRDIIKPDPPKNLQMKPLKNSQVEVSWEYPDSWST | |
| | PHSYFSLKFFVRIQRKKEKMKETEEGCNQKGAFLVEKTSTEVQ | |
| | CKGGNVCVQAQDRYYNSSCSKWACVPCRVRSVPGVGVPGVGRV | |
| | IPVSGPARCLSQSRNLLKTTDDMVKTAREKLKHYSCTAEDIDH | |
| | EDITRDQTSTLKTCLPLELHKNESCLATRETSSTTRGSCLPPQ | |
| | KTSLMMTLCLGSIYEDLKMYQTEFQAINAALQNHNHQQIILDK | |
| | GMLVAIDELMQSLNHNGETLRQKPPVGEADPYRVKMKLCILLH | |
| | AFSTRVVTINRVMGYLSSA | |
| 12T4 | MCPQKLTISWFAIVLLVSPLMAMWELEKDVYVVEVDWTPDAPG | 13 |
| | ETVNLTCDTPEEDDITWTSDQRHGVIGSGKTLTITVKEFLDAG | |
| | QYTCHKGGETLSHSHLLLHKKENGIWSTEILKNFKNKTFLKCE | |
| | APNYSGRFTCSWLVQRNMDLKFNIKSSSSSPDSRAVTCGMASL | |
| | SAEKVTLDQRDYEKYSVSCQEDVTCPTAEETLPIELALEARQQ | |
| | NKYENYSTSFFIRDIIKPDPPKNLQMKPLKNSQVEVSWEYPDS | |
| | WSTPHSYFSLKFFVRIQRKKEKMKETEEGCNQKGAFLVEKTST | |
| | EVQCKGGNVCVQAQDRYYNSSCSKWACVPCRVRSVPGVGVPGV | |
| | GRVIPVSGPARCLSQSRNLLKTTDDMVKTAREKLKHYSCTAED | |
| | IDHEDITRDQTSTLKTCLPLELHKNESCLATRETSSTTRGSCL | |
| | PPQKTSLMMTLCLGSIYEDLKMYQTEFQAINAALQNHNHQQII | |
| | LDKGMLVAIDELMQSLNHNGETLRQKPPVGEADPYRVKMKLCI | |
| | LLHAFSTRVVTINRVMGYLSSAGSGEGRGSLLTCGDVEENPGP | |
| | LRSPVTMDQHTLDVEDTADARHPAGTSCPSDAALLRDTGLLAD | |
| | AALLSDTVRPTNAALPTDAAYPAVNVRDREAAWPPALNFCSRH | |
| | PKLYGLVALVLLLLIAACVPIFTRTEPRPALTITTSPNLGTRE | |
| | NNADQVTPVSHIGCPNTTQQGSPVFAKLLAKNQASLCNTTLNW | |
| | HSQDGAGSSYLSQGLRYEEDKKELVVDSPGLYYVFLELKLSPT | |
| | FTNTGHKVQGWVSLVLQAKPQVDDFDNLALTVELFPCSMENKL | |
| | VDRSWSQLLLLKAGHRLSVGLRAYLHGAQDAYRDWELSYPNTT | |
| | SFGLFLVKPDNPWE | |

In Table 1, the IL-12 sequence is shown in plain Roman text, the 4-1BBL sequence is shown in bold text, the T2A sequence is shown in underlined text, and the GS linker sequence is shown in double underlined text.

Cloning—Nucleic acids for use as cloned gene inserts were ordered from a commercial source (Twist Bioscience) and comprised a nucleotide sequence encoding mouse or human 4-1BBL, a GS linker, and mouse or human IL12. The GS linker (“tether sequence”) consisted of a 25 amino acid repeat of Gly-Gly-Gly-Gly-Ser. The backbone plasmid (pUNO MCS, Invivogen, puno1-mcs) and the gene insert nucleic acids were double digested using the restriction enzymes NheI-HF and BamHI-HF (New England Biolobs catalog numbers R3131S and R3136S). The products of the restriction digests were separated using gel electrophoresis on an agarose gel (600 mg agarose (Invitrogen catalog number 11553277), 60 mL 1×TAE buffer (Biorad catalog number 1610743), and 6 μL ethidium bromide (Sigma Aldrich catalog number E7637). Gel bands containing the restriction products to be used for cloning were excised and extracted using a QIAquick Gel Extraction Kit (Qiagen catalog number 28704). After determining the concentrations of extracted nucleic acids using a spectrophotometer (NanoDrop, ThermoFisher), 100 ng of insert was mixed with 50 ng of the backbone and ligated using T4 Ligase (New England Biolabs catalog number M0202S). The ligation product was transformed into Subcloning Efficiency DH5alpha Competent Cells (ThermoFisher catalog number 18265017). Small cultures were grown and miniprepped (Qiagen catalog number 27104), and the nucleotide sequences were confirmed by the Johns Hopkins University Sequencing Core. Samples were maxiprepped (Zymo Research catalog number D4202) to provide nucleic acid constructs for transfecting cells. Exemplary nucleic acid constructs are shown in FIGS. 1A, 1B, 1C, and 4A.

Polymer Synthesis—PBAEs were synthesized based on previously published protocols (see Green (2008) “A Combinatorial Polymer Library Approach Yields Insight into Nonviral Gene Delivery” Acc. Chem. Res. 41 (6): 749-59, incorporated herein by reference). Briefly, seven PBAEs were synthesized by Michael Addition reactions: 4-4-6, Apr. 4, 2027, 4-5-6, 4-5-7, Apr. 5, 2027, Apr. 5, 1939, and 5-3-6, with these numerical codes denoting the polymer backbones, sidechains, and end caps in that order (see, e.g., Karlsson (2020) “Poly(Beta-Amino Ester) s as Gene Delivery Vehicles: Challenges and Opportunities” Expert Opin Drug Deliv 17 (10): 1395-1410, incorporated herein by reference).

Cells—B16-F10 mouse melanoma cells were obtained from ATCC (ATCC, catalog no. CRL-6475). Cells were cultured in DMEM (Thermo Fisher Scientific, catalog no. 11965092) supplemented with 10% fetal bovine serum (Milliporesigma, catalog no. F4135-500ML) and 1% penicillin/streptomycin (Thermo Fisher Scientific, catalog no. 15140122) at 37° C. and a humidified environment.

In vitro NP formulation and transfection—B16-F10 cells were seeded at a cell density of 10,000 cells per well on day 0. On day 1, PBAEs and DNA were diluted in 25 mM sodium acetate, mixed, and self-assembled within minutes to form PBAE NPs. 600 ng of DNA were delivered to B16-F10 melanoma cells at 30, 60, and 90 w/w (weight/weight polymer mass to DNA mass ratio) in 20 μL of NPs. After 2 hours, all media was changed. On day 2, cell viability was measured via MTS assay (see below), and on day 3, cells or cell lysate were harvested for ELISA or flow cytometry for further analysis.

Viability (MTS)—24 hours following cell transfection, cell media in each well was removed and replaced with a 100 μL of a 1:10 ratio of MTS assay buffer (Promega, catalog no. G3582) to cell media. The plate was then placed in an incubator at 37° C. After one hour, absorbance was measured at 490 nm wavelength.

Flow cytometry and antibody staining—Cells were washed with 200 μL of PBS and detached from the plate using 30 μL of Trypsin-EDTA (Thermo Fischer Scientific, catalog no. 25300054). The cells were then quenched with 170 μL of FACS buffer (PBS+10% FBS) and transferred to a v-bottom plate to be centrifuged at 300 rcf for 5 minutes at 4° C. The supernatant was removed, and cells were resuspended in 100 μL of FACS buffer. Cells were stained with antibodies against 4-1BBL and IL-12. Antibodies are listed in Table 2.

TABLE 2
Antibody Table

| | | | | Catalog | |
|---|---|---|---|---|---|
| Fluorophore | Target | Clone | Manufacturer | No. | Dilution |
| PE | anti-mouse 4-1BB | 5F4 | Biolegend | 107105 | 80 |
| | Ligand | | | | |
| ALEXA | Anti-mouse IL-12/ | C15.6 | Biolegend | 505214 | 100 |
| FLUOR | IL-23 p40 | | | | |

ELISA—Cells were lysed using InstantOne ELISA Cell Lysis Buffer (ThermoFisher catalog number IOCLB1), and both the transfection media and cell lysates were assayed by the ELISA. For mouse IL-12 detection, cell supernatant was harvested 48 hours after cell transfection and measured via ELISA (Biolegend, catalog no. 433604). For mouse IFNγ detection, mouse splenocytes were harvested from the spleen of one C57BL/6 mouse. The spleen was pressed through a 70 μm filter, incubated for one minute with ACK buffer (Quality Biological, catalog no. 118-156-721) for red blood cell (RBC) lysis, diluted in PBS, and passed through a 40 μm filter. Cells were centrifuged, resuspended in complete DMEM and 200K splenocytes co-cultured with B16-F10 cells 24 hours after transfection. Cell supernatant was harvested 24 hours after co-culture and measured via ELISA (Biolegend, catalog no. 430804) according to the manufacturer's instructions. For 4-1BBL detection, two days after transfection of B16 F10 cells, the cells were harvested, stained with anti-41BBL antibody, and analyzed using flow cytometry. Cells were first washed with 100 μL PBS (Gibco catalog number 10-010-023), then trypsinized with 30 μL Trypsin (ThermoFisher catalog number 25300054). Cells were collected by centrifugation and the supernatant was removed. The cells were resuspended in FACS buffer, 2% FBS (Millipore Sigma catalog number F4135) in PBS. The cells were then stained with PE anti-mouse 4-1BB Ligand (Biolegend catalog number 107105) according to the Biolegend antibody staining protocol.

Co-Culture Experiment to detect IFNγ production—The day following cell transfection with nanoparticles, mouse splenocytes were isolated and added to the cells. Spleens were pressed and washed with PBS through a 70 μm cell strainer and centrifuged at 500×g for 5 minutes. The red blood cells were lysed with 1 mL ACK buffer (Quality Biological catalog number 118-156-721) and diluted with PBS after 1 minute. The lysate comprising lysed red blood cells and intact splenocyte cells was pressed through a 40-μm cell strainer and centrifuged at 500×g for 5 minutes. Cells were counted and 200,000 splenocytes were added in a volume of 100 μL to each well. 48 hours after transfection, 100 μL of cell media were harvested for an IFNγ ELISA (Biolegend catalog number 88-7314-88).

NP Sizing and Characterization—NPs were prepared with 0.2 mg/mL DNA in 75 μL, at 30 w/w of 5-3-6 PBAE polymer. After 10 minutes of complexation, NPs were diluted 1:100 in 0.1×PBS and measured on a Malvern ZetaSizer Pro. For each NP formulation, n=4 replicates were performed for both size and Zeta Potential.

Transmission Electron Microscope (TEM) Imaging—5-3-6 NPs were formulated in 25 mM sodium acetate and diluted in deionized water to a 0.2 mg/mL DNA concentration. Samples were transferred onto a carbon film 400 mesh copper grid (Electron Microscopy Sciences; Hatfield, PA), and dried at room temperature for 5-6 hours. Grids were washed with deionized water to remove excess salts, followed by staining in 1% Uranyl Acetate solution (Electron Microscopy Sciences; Hatfield, PA). The grids were then dried overnight, and samples were imaged on the Hitachi 7600 transmission electron microscope (TEM) (Hitachi High-Tech; Tokyo, Japan).

Survival study & monitoring—All animal studies were performed within the guidelines of the Johns Hopkins Animal Care and Use Committee under approved protocol numbers MO23E357 and MO24M284. 300K B16-F10 cells were implanted subcutaneously into the flanks of seven groups of C57BL/6J mice (t=0). The groups were 1) PBAE NPs encoding 4-1BBL and IL-12 (distinct plasmids), 2) PBAE NPs encoding 4T12, 3) PBAE NPs encoding 12T4, 4) PBAE NPs encoding 4G512, 5) PBAE NPs encoding luciferase, 6) maximum tolerable dose (MTD) of IL-12 protein, and 7) a low dose (3 ng/kg) of IL-12 protein. 5-3-6 was used for the polymer in the PBAE NP groups. All groups received 100 μg of systemic anti-PD1. 5-3-6 PBAE polymers were formulated with 0.2 mg/mL DNA, at 30 w/w in 2.7 mLs to form NPs. All dilutions were done with Magnesium Acetate (Sigma Aldrich, catalog no. 63052-100ML) at pH 7.5 and 25 mM. Recombinant IL-12 protein (Thermo Fisher Scientific, catalog no. 210-12-10UG) was diluted in PBS. 50 μL of PBAE NPs were injected intratumorally (t=7, 9, 11 in bicistronic study, t=9, 11, 16, 18 in Nanoplasmid study). IL-12 protein groups were delivered on t=7, 11, 14, 18, 21, and 25 systemically, either by retroorbital or tail vein injection. This IL-12 dosing strategy was selected to be delivered systemically and twice weekly based on previous studies reported in literature23,44. Every two days, surface area was measured as tumor length×width. Mice were euthanized when the area of the tumor exceeded 200 mm² or for moribund states.

Tumor Rechallenge—In the survival study comparing different bicistronic plasmids, seven mice exhibited regressed tumors. These mice were rechallenged with 300K B16-F10 cells on the left flank, opposite from the initial study. Age matched C57BL/6J mice were similarly implanted with B16-F10 cells. Every two days, surface area was measured as described above, and mice were euthanized when the tumor exceeded 200 mm² or for moribund states.

### Example 1

During the development of embodiments of the technology described herein, experiments were conducted to detect IFNγ production by mouse splenocytes contacted by cells transfected by dual transgene constructs using nanoparticles. The following DNA constructs were delivered in gene delivery particles to B16-F10 melanoma cells: a single plasmid encoding GFP (“GFP”); a single plasmid encoding mouse 41BBL (“41BBL”); a single plasmid encoding mouse IL12 (“IL12”); a mixture of a first plasmid encoding mouse 41BBL and a second plasmid encoding mouse IL12 (“½ 41BBL ½ IL12”); a plasmid encoding mouse 41BBL linked by an autocatalytic linker to mouse IL12 (“41BBL T2A IL12”); and a plasmid encoding mouse 41BBL linked by a tether sequence (GS linker) to mouse IL12 (“mTether (41BBL G4S IL12)”). In 41BBL T2A IL12, the nucleic acid comprises an ordered arrangement of the three coding sequences for mouse 41BBL, T2A, and mouse IL12 in order from 5′ to 3′ to produce an amino acid sequence of mouse 41BBL, T2A, and mouse IL12 in order from the amino terminus to the carboxyl terminus of the polypeptide. Similarly, in mTether (41BBL G4S IL12), the nucleic acid comprises an ordered arrangement of the three coding sequences for mouse 41BBL, G4S, and mouse IL12 in order from 5′ to 3′ to produce an amino acid sequence of mouse 41BBL, G4S, and mouse IL12 in order from the amino terminus to the carboxyl terminus of the polypeptide.

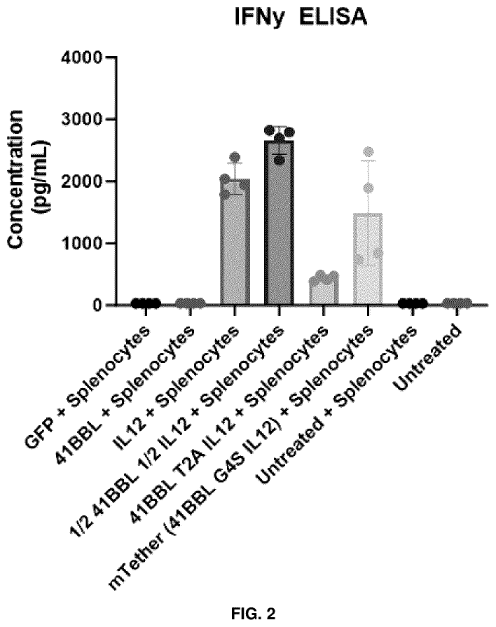

B6 mouse splenocytes were then added to produce a coculture for evaluating immune responses. Sample “41BBL T2A IL12” demonstrates a “skip” approach in which the nucleic acid construct expresses two separate signal 2 (1BBL) and signal 3 (IL12) proteins from the same DNA molecule. Sample “mTether (41BBL G4S IL12)” demonstrates the protein “fusion” approach using an amino acid tether of glycine and serine linking the signal 2 (1BBL) and signal 3 (IL12) proteins for expression as a single polypeptide. Both novel constructs led to immune activation as indicated by the elevated interferon gamma levels compared to the controls. FIG. 2.

### Example 2

During the development of embodiments of the technology provided herein, experiments were conducted to test additional nucleic acid constructs comprising coding sequences for 41BBL and IL12 in which both sequences were terminated by a stop codon. Bicistronic constructs were produced in which both transgene coding sequences comprised a stop codon, and monocistronic constructs were produced in which the single transgene coding sequence comprised a stop codon. In particular, the following monocistronic constructs were designed, produced in the backbone plasmid (pUNO MCS, Invivogen, puno1-mcs), and tested: pUNO GFP, pUNO 41BBL, and pUNO IL12. All monocistronic plasmids comprised the indicated GFP, mouse 41BBL, or mouse IL12 coding sequence terminated by a stop codon. The following bicistronic constructs were designed, produced in the backbone plasmid (pUNO MCS, Invitrogen, puno1-mcs): pUNO IL12 T2A 41BBL; pUNO 41BBL T2A IL12; pUNO 41BBL G4S IL12 (mouse); and pUNO 41BBL G4S IL12 (human).

In pUNO IL12 T2A 41BBL, the nucleic acid comprises an ordered arrangement of the three coding sequences for mouse IL12, T2A, and mouse 41BBL in order from 5′ to 3′ to produce an amino acid sequence of mouse IL12, T2A, and mouse 41BBL in order from the amino terminus to the carboxyl terminus of the polypeptide and in which each of mouse IL12 and mouse 41BBL nucleotide sequences have a stop codon. In pUNO 41BBL T2A IL12, the nucleic acid comprises an ordered arrangement of the three coding sequences for mouse 41BBL, T2A, and mouse IL12 in order from 5′ to 3′ to produce an amino acid sequence of mouse 41BBL, T2A, and mouse IL12 in order from the amino terminus to the carboxyl terminus of the polypeptide and in which each of mouse 41BBL and mouse IL12 nucleotide sequences have a stop codon. In pUNO 41BBL G4S IL12 (mouse), the nucleic acid comprises an ordered arrangement of the three coding sequences for the mouse homolog of 41BBL, G4S, and the mouse homolog of IL12 in order from 5′ to 3′ to produce an amino acid sequence of mouse 41BBL, G4S, and mouse IL12 in order from the amino terminus to the carboxyl terminus of the polypeptide and in which each of mouse 41BBL and mouse IL12 nucleotide sequences have a stop codon. Similarly, in pUNO 41BBL G4S IL12 (human), the nucleic acid comprises an ordered arrangement of the three coding sequences for the human homolog of 41BBL, G4S, and the human homolog of IL12 in order from 5′ to 3′ to produce an amino acid sequence of human 41BBL, G4S, and human IL12 in order from the amino terminus to the carboxyl terminus of the polypeptide and in which each of human 41BBL and human IL12 nucleotide sequences have a stop codon.

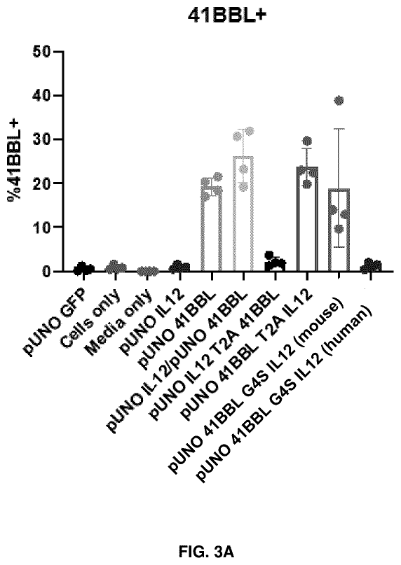

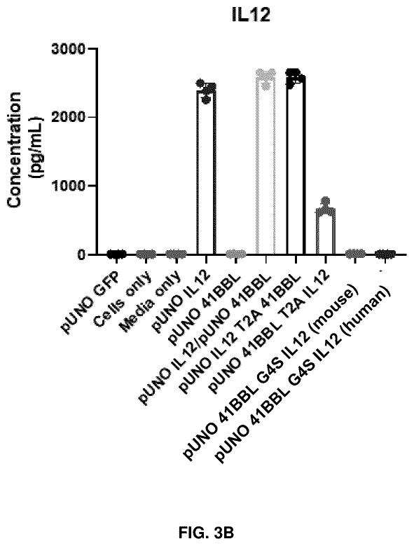

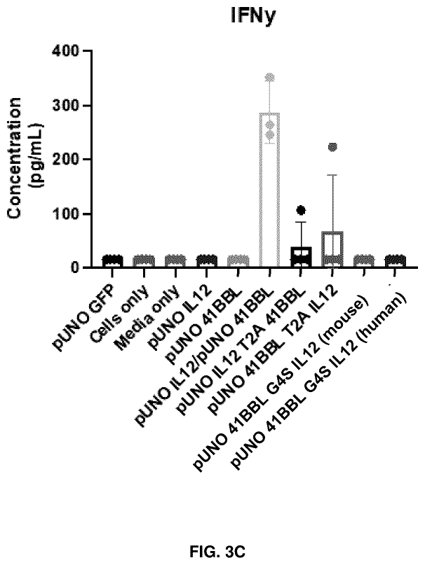

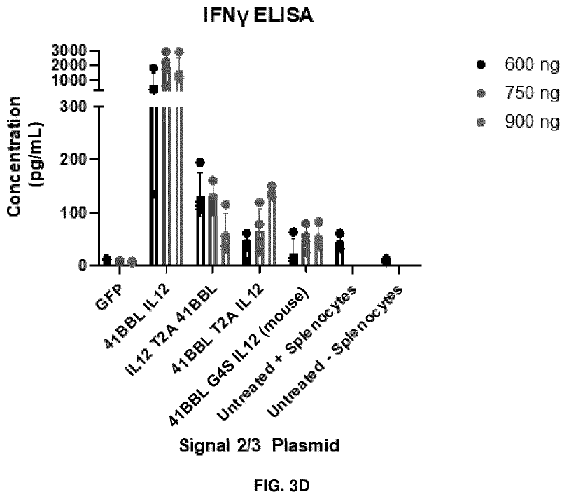

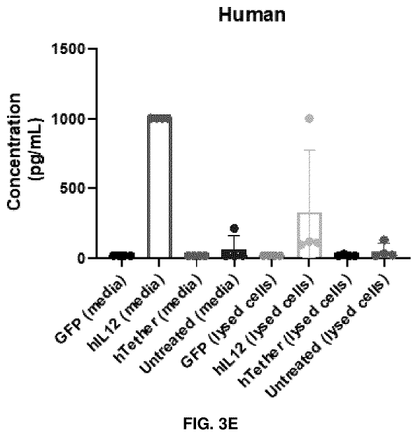

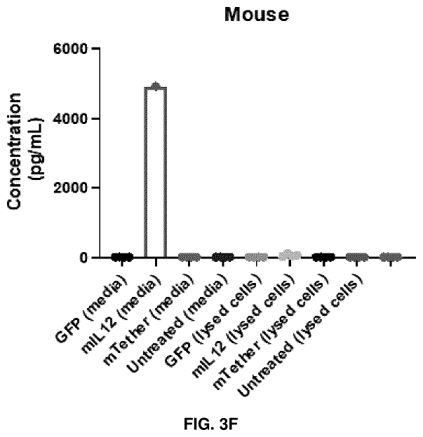

As in Example 1, experiments were conducted to detect IFNγ production by mouse splenocytes contacted by B16 F10 mouse melanoma cells transfected by the monocistronic and bicistronic transgene constructs comprising stop codons after each coding sequence using nanoparticles. The DNA constructs were delivered in gene delivery particles to B16-F10 melanoma cells as described in the methods and Example 1 above, and B6 mouse splenocytes were then added to produce a coculture for evaluating immune responses (e.g., production of interferon gamma). FIG. 3A-3F. As shown in FIG. 3A, antibody staining for 4-1BBL demonstrates expression. FIG. 3B shows ELISA testing indicated IL12 expression after dual transgene construct delivery. FIG. 3C shows ELISA testing for IFNγ demonstrated low levels of immune response for all constructs except for 4-1BBL and IL-12 delivered on and expressed from separate plasmids. FIG. 3D shows that data from experiments in which doses of dual transgene DNA constructs were varied to improve IFNγ expression. FIG. 3E and FIG. 3F show IL12 ELISA of cell media and of cells after lysis of the tethered dual transgene constructs comprising human and mouse 41BBL and IL12, respectively. All experiments were performed in B16 F10 melanoma cells using the PBAE polymer 4-4-6 at 60:1 w/w ratio of polymer to DNA. These data indicated that bicistronic constructs comprising a stop codon after each signal 2 (41BBL) and signal 3 (IL12) transgene were much less effective that the bicistronic constructs comprising signal 2 (41BBL) and signal 3 (IL12) linked by an autocatalytic linker or a tether. Without being limited by theory, it is contemplated that linking signal 2 (41BBL) and signal 3 (IL12) linked by an autocatalytic linker or a tether increases colocalization of signal 2 (41BBL) and signal 3 (IL12) at the cell surface where they are more effective in inducing an immune response than when signal 2 (41BBL) and signal 3 (IL12) are expressed in the cytoplasm as separate polypeptides.

### Example 3

During the development of embodiments of the technology described herein, experiments were conducted to engineer bicistronic plasmids for co-expression of 4-1BBL and IL-12. Six plasmid constructs containing 4-1BBL and IL-12 were produced. Two constructs comprised the 2A peptide sequence, and four constructs comprised the (G4S)n peptide tether (FIG. 4A and FIG. 4B. See also Table 1). T2A, derived from thosea asigna virus, was chosen as the 2A sequence since it has the highest cleavage efficiency among the 2A peptides, thus ensuring that the proteins would not remain linked. In plasmids with the T2A sequence, one was formed with 4-1BBL at the N-terminus (4T12), and the other was formed with IL-12 at the N-terminus (12T4) to explore if sequence order affected expression and efficacy of tumor reprogramming. In the plasmids encoding the fusion protein with the (G4S)n tether, four lengths of the (G4S)n repeat (n=2, 3, 5, 7), corresponding to 10, 15, 25, and 35 amino acids were tested to evaluate which tether length resulted in the most effective tumor reprogramming. These constructs were labeled as 4Gn12 and designed so the (G4S)n repeat is positioned between the C terminus of 4-1BBL and the N terminus of IL-12 in order to enforce extracellular expression of IL-12, as 4-1BBL is a Type 2 transmembrane protein with a C-terminal extracellular domain22. All plasmids were cloned into the pUNO1 backbone.

### Example 4

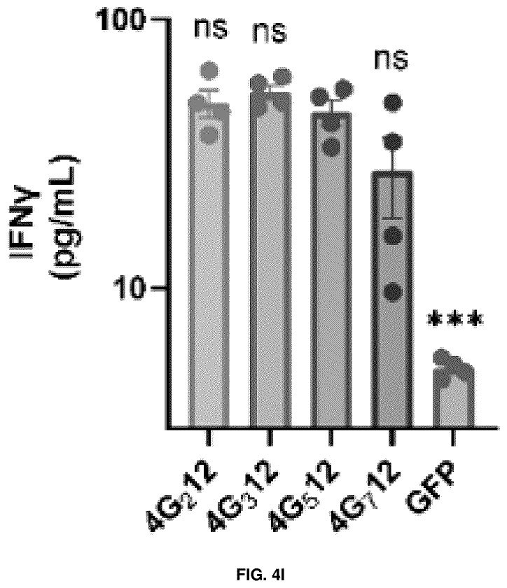

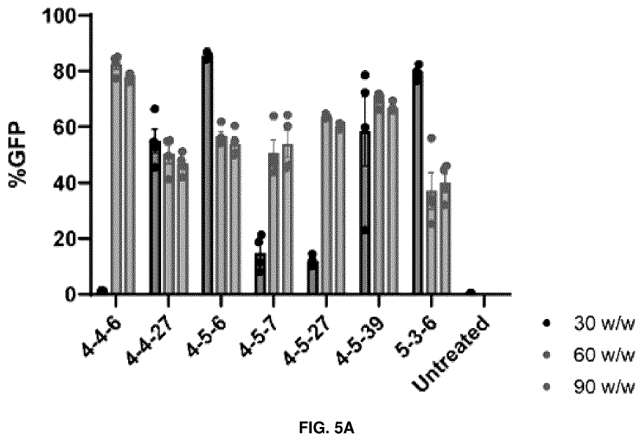

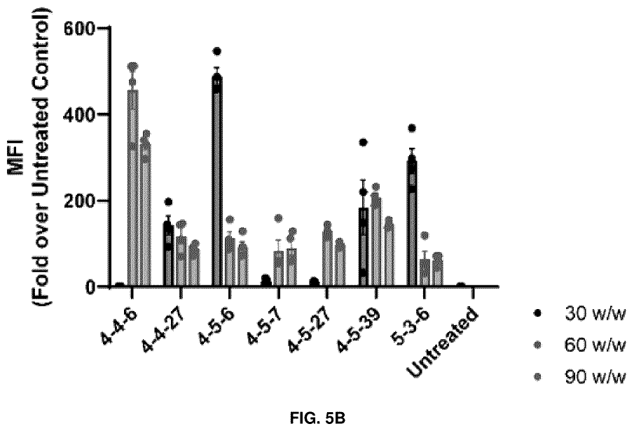

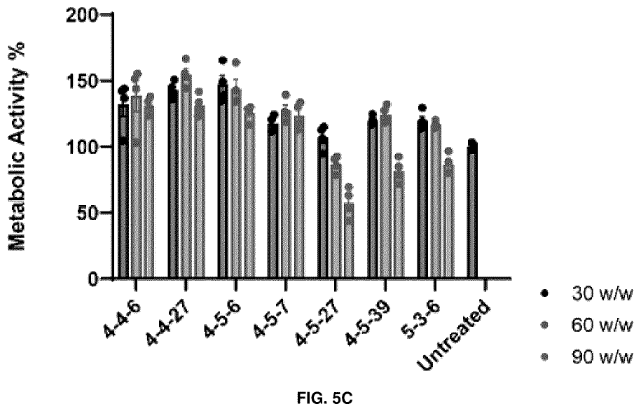

During the development of embodiments of the technology described herein, experiments were conducted to optimize the PBAE NP formulation to maximize the efficiency of in vitro transfection. B16-F10 melanoma cells were selected to test the transfection efficiency of the engineered bicistronic plasmids because the tAPC platform has previously been validated in this model. GFP plasmid delivery via seven PBAE NPs was evaluated to select a formulation that demonstrated robust transfection that would facilitate downstream analysis of bicistronic plasmid protein expression. Of the tested NPs, the 60 w/w 4-4-6 NP condition was chosen for further in vitro testing because this condition demonstrated 82±2% (mean±SEM) transfection efficiency (FIG. 5A), mean fluorescence intensity of ~460-fold over untreated cells (FIG. 5B), and relative metabolic activity that was ~140% of the untreated cells (FIG. 5C).

### Example 5

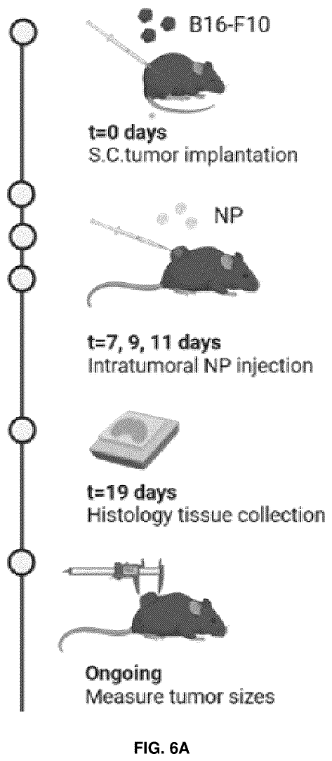

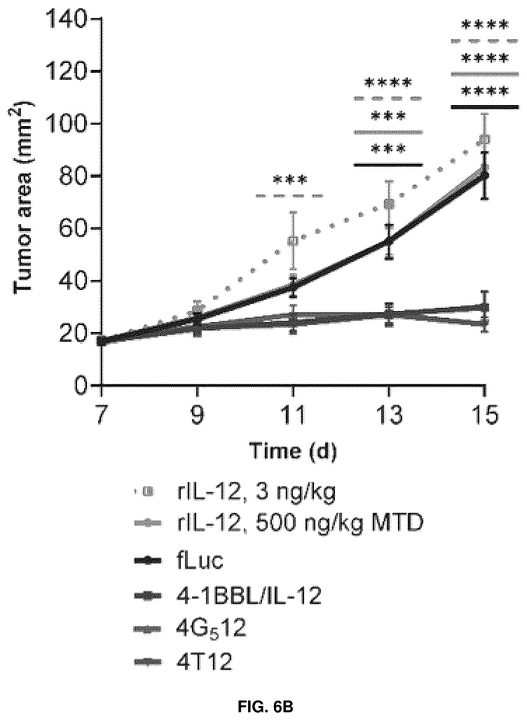

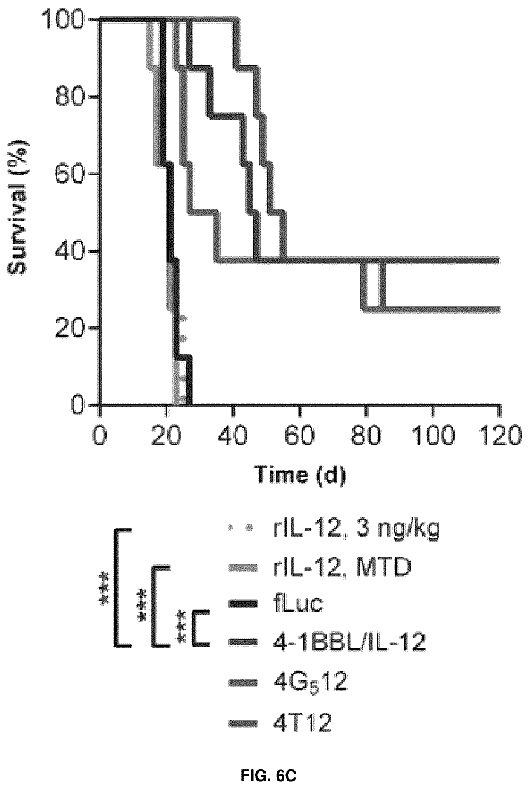

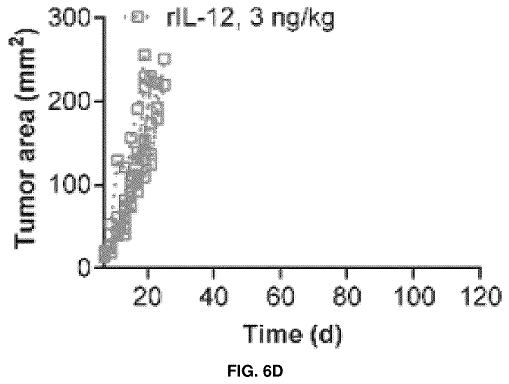

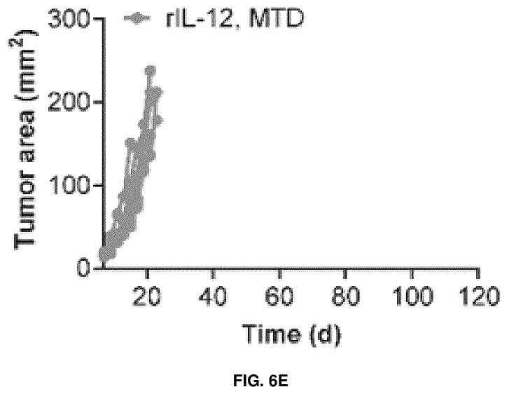

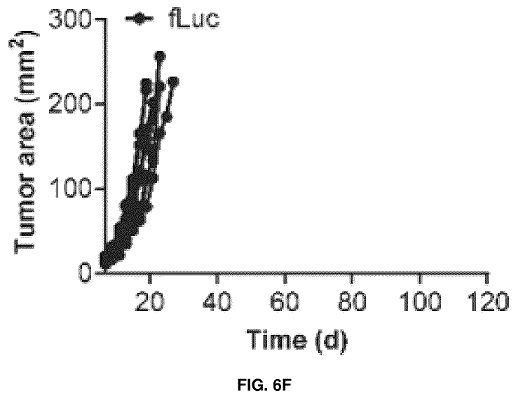

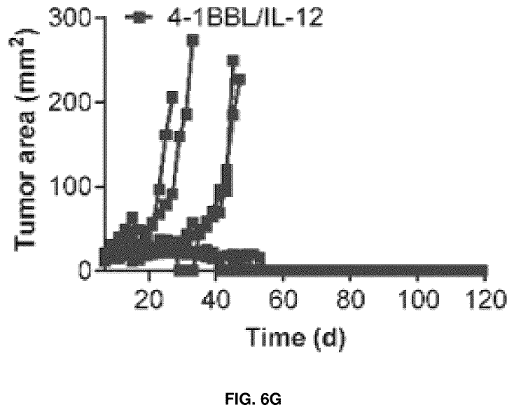

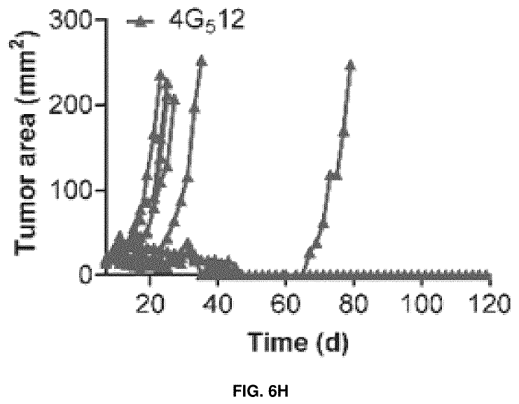

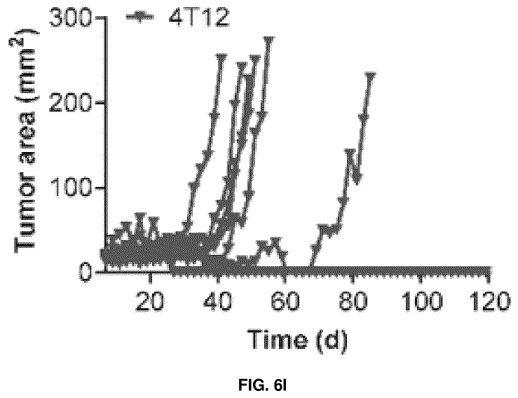

During the development of embodiments of the technology described herein, experiments were conducted to use bicistronic plasmids to co-express 4-1BBL and IL-12 in multiple formats. Embodiments of the plasmids described herein were assessed in vitro to compare 4-1BBL and IL-12 expression when the two immunomodulatory signals were delivered on a single plasmid or on separate plasmids (FIG. 4C). Flow cytometry analysis of transfected cells showed that 20±6% (mean±SEM) of cells were 4-1BBL+ when 4-1BBL and IL-12 were delivered on separate plasmids (4-1BBL/IL-12) (FIG. 6D). Notably, when compared to co-delivery of 4-1BBL and IL-12 individual plasmids, the percentage of 4-1BBL+ cells was not significantly different following transfection with 4G512 (18±10%) or 4T12 (22±4%). However, the percentage of 4-1BBL+ cells was significantly lower in the 12T4 condition (0.2±0.1%) compared to co-delivery of the two plasmids. Detailed statistical analysis is provided in Table 3.

TABLE 3
Statistical analysis

| Statistical | | | | Adjusted | |
|---|---|---|---|---|---|
| test | Measurement | Comparison | Summary | P Value | Figure |
| One-way | %4-1BBL⁺ | 4-1BBL/IL-12 vs. 4-1BBL | *** | 0.001 | 4D |
| ANOVA | Expression | | | | |
| (Dunnett post | | | | | |
| hoc test) | | | | | |
| One-way | %4-1BBL⁺ | 4-1BBL/IL-12 vs. IL12 | * | 0.0214 | 4D |
| ANOVA | Expression | | | | |
| (Dunnett post | | | | | |
| hoc test) | | | | | |
| One-way | %4-1BBL⁺ | 4-1BBL/IL-12 vs. 4G₅12 | ns | 0.9998 | 4D |
| ANOVA | Expression | | | | |
| (Dunnett post | | | | | |
| hoc test) | | | | | |
| One-way | %4-1BBL⁺ | 4-1BBL/IL-12 vs. 4T12 | ns | 0.9996 | 4D |
| ANOVA | Expression | | | | |
| (Dunnett post | | | | | |
| hoc test) | | | | | |
| One-way | %4-1BBL⁺ | 4-1BBL/IL-12 vs. 12T4 | * | 0.0208 | 4D |
| ANOVA | Expression | | | | |
| (Dunnett post | | | | | |
| hoc test) | | | | | |
| One-way | %4-1BBL⁺ | 4-1BBL/IL-12 vs. GFP | * | 0.0214 | 4D |
| ANOVA | Expression | | | | |
| (Dunnett post | | | | | |
| hoc test) | | | | | |
| One-way | %4-1BBL⁺ | 4-1BBL/IL-12 vs. Untreated | * | 0.02 | 4D |
| ANOVA | Expression | | | | |
| (Dunnett post | | | | | |
| hoc test) | | | | | |
| One-way | % IL-12⁺ | 4-1BBL/IL-12 vs. 4-1BBL | ns | >0.9999 | 4E |
| ANOVA | Expression | | | | |
| (Dunnett post | | | | | |
| hoc test) | | | | | |
| One-way | % IL-12⁺ | 4-1BBL/IL-12 vs. IL12 | ns | >0.9999 | 4E |
| ANOVA | Expression | | | | |
| (Dunnett post | | | | | |
| hoc test) | | | | | |
| One-way | % IL-12⁺ | 4-1BBL/IL-12 vs. 4G512 | *** | 0.0008 | 4E |
| ANOVA | Expression | | | | |
| (Dunnett post | | | | | |
| hoc test) | | | | | |
| One-way | % IL-12⁺ | 4-1BBL/IL-12 vs. 4T12 | ns | >0.9999 | 4E |
| ANOVA | Expression | | | | |
| (Dunnett post | | | | | |
| hoc test) | | | | | |
| One-way | % IL-12⁺ | 4-1BBL/IL-12 vs. 12T4 | ns | >0.9999 | 4E |
| ANOVA | Expression | | | | |
| (Dunnett post | | | | | |
| hoc test) | | | | | |
| One-way | % IL-12⁺ | 4-1BBL/IL-12 vs. GFP | ns | >0.9999 | 4E |
| ANOVA | Expression | | | | |
| (Dunnett post | | | | | |
| hoc test) | | | | | |
| One-way | % IL-12⁺ | 4-1BBL/IL-12 vs. Untreated | ns | >0.9999 | 4E |
| ANOVA | Expression | | | | |
| (Dunnett post | | | | | |
| hoc test) | | | | | |
| One-way | IL-12 | 4-1BBL/IL-12 vs. 4-1BBL | *** | 0.0002 | 4F |
| ANOVA | Concentration | | | | |
| (Dunnett post | | | | | |
| hoc test) | | | | | |
| One-way | IL-12 | 4-1BBL/IL-12 vs. IL12 | ns | 0.7648 | 4F |
| ANOVA | Concentration | | | | |
| (Dunnett post | | | | | |
| hoc test) | | | | | |
| One-way | IL-12 | 4-1BBL/IL-12 vs. 4G512 | *** | 0.0002 | 4F |
| ANOVA | Concentration | | | | |
| (Dunnett post | | | | | |
| hoc test) | | | | | |
| One-way | IL-12 | 4-1BBL/IL-12 vs. 4T12 | ns | 0.074 | 4F |
| ANOVA | Concentration | | | | |
| (Dunnett post | | | | | |
| hoc test) | | | | | |
| One-way | IL-12 | 4-1BBL/IL-12 vs. 12T4 | ns | 0.4154 | 4F |
| ANOVA | Concentration | | | | |
| (Dunnett post | | | | | |
| hoc test) | | | | | |
| One-way | IL-12 | 4-1BBL/IL-12 vs. GFP | *** | 0.0002 | 4F |
| ANOVA | Concentration | | | | |
| (Dunnett post | | | | | |
| hoc test) | | | | | |
| One-way | IL-12 | 4-1BBL/IL-12 vs. Untreated | *** | 0.0002 | 4F |
| ANOVA | Concentration | | | | |
| (Dunnett post | | | | | |
| hoc test) | | | | | |
| One-way | IFNγ | 4-1BBL/IL-12 vs. 4G512 | **** | <0.0001 | 4H |
| ANOVA | Concentration | | | | |
| (Dunnett post | | | | | |
| hoc test) | | | | | |
| One-way | IFNγ | 4-1BBL/IL-12 vs. 4T12 | **** | <0.0001 | 4H |
| ANOVA | Concentration | | | | |
| (Dunnett post | | | | | |
| hoc test) | | | | | |
| One-way | IFNγ | 4-1BBL/IL-12 vs. 12T4 | **** | <0.0001 | 4H |
| ANOVA | Concentration | | | | |
| (Dunnett post | | | | | |
| hoc test) | | | | | |
| One-way | IFNγ | 4-1BBL/IL-12 vs. GFP | **** | <0.0001 | 4H |
| ANOVA | Concentration | | | | |
| (Dunnett post | | | | | |
| hoc test) | | | | | |
| One-way | IFNγ | 4G512 vs. GFP | *** | 0.0004 | 4I |
| ANOVA | Concentration | | | | |
| (Dunnett post | | | | | |
| hoc test) | | | | | |
| One-way | IFNγ | 4G512 vs. 4G212 | ns | 0.9704 | 4I |
| ANOVA | Concentration | | | | |
| (Dunnett post | | | | | |
| hoc test) | | | | | |
| One-way | IFNγ | 4G512 vs. 4G312 | ns | 0.6529 | 4I |
| ANOVA | Concentration | | | | |
| (Dunnett post | | | | | |
| hoc test) | | | | | |
| One-way | IFNγ | 4G512 vs. 4G712 | ns | 0.1085 | 4I |
| ANOVA | Concentration | | | | |
| (Dunnett post | | | | | |
| hoc test) | | | | | |
| Two-way | Tumor Area | t = 7 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 9 | ns | 0.9853 | 6B |
| ANOVA | | Signal 2 + Signal 3 vs. fLuc | | | |
| (Dunnett post | | | | | |
| hoc test) | | | | | |
| Two-way | Tumor Area | t = 9 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 9 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 9 | ns | 0.8192 | 6B |
| ANOVA | | Signal 2 + Signal 3 vs. fLuc | | | |
| (Dunnett post | | | | | |
| hoc test) | | | | | |
| Two-way | Tumor Area | t = 9 | ns | 0.971 | 6B |
| ANOVA | | Signal 2 + Signal 3 vs. fLuc | | | |
| (Dunnett post | | | | | |
| hoc test) | | | | | |
| Two-way | Tumor Area | t = 11 | ns | 0.2341 | 6B |
| ANOVA | | Signal 2 + Signal 3 vs. fLuc | | | |
| (Dunnett post | | | | | |
| hoc test) | | | | | |
| Two-way | Tumor Area | t = 11 | ns | 0.992 | 6B |
| ANOVA | | Signal 2 + Signal 3 vs. fLuc | | | |
| (Dunnett post | | | | | |
| hoc test) | | | | | |
| Two-way | Tumor Area | t = 11 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 11 | *** | 0.0001 | 6B |
| ANOVA | | Signal 2 + Signal 3 vs. fLuc | | | |
| (Dunnett post | | | | | |
| hoc test) | | | | | |
| Two-way | Tumor Area | t = 11 | ns | 0.1833 | 6B |
| ANOVA | | Signal 2 + Signal 3 vs. fLuc | | | |
| (Dunnett post | | | | | |
| hoc test) | | | | | |
| Two-way | Tumor Area | t = 13 | *** | 0.0009 | 6B |
| ANOVA | | Signal 2 + Signal 3 vs. fLuc | | | |
| (Dunnett post | | | | | |
| hoc test) | | | | | |
| Two-way | Tumor Area | t = 13 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 13 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 13 | **** | <0.0001 | 6B |
| ANOVA | | Signal 2 + Signal 3 vs. fLuc | | | |
| (Dunnett post | | | | | |
| hoc test) | | | | | |
| Two-way | Tumor Area | t = 13 | *** | 0.0009 | 6B |
| ANOVA | | Signal 2 + Signal 3 vs. fLuc | | | |
| (Dunnett post | | | | | |
| hoc test) | | | | | |
| Two-way | Tumor Area | t = 15 | **** | <0.0001 | 6B |
| ANOVA | | Signal 2 + Signal 3 vs. fLuc | | | |
| (Dunnett post | | | | | |
| hoc test) | | | | | |
| Two-way | Tumor Area | t = 15 | ns | >0.9999 | 6B |
| ANOVA | | Signal 2 + Signal 3 vs. fLuc | | | |
| (Dunnett post | | | | | |
| hoc test) | | | | | |
| Two-way | Tumor Area | t = 15 | ns | 0.8476 | 6B |
| ANOVA | | Signal 2 + Signal 3 vs. fLuc | | | |
| (Dunnett post | | | | | |
| hoc test) | | | | | |
| Two-way | Tumor Area | t = 15 | **** | <0.0001 | 6B |
| ANOVA | | Signal 2 + Signal 3 vs. fLuc | | | |
| (Dunnett post | | | | | |
| hoc test) | | | | | |
| Two-way | Tumor Area | t = 15 | **** | <0.0001 | 6B |
| ANOVA | | Signal 2 + Signal 3 vs. fLuc | | | |
| (Dunnett post | | | | | |
| hoc test) | | | | | |
| Kaplan Meier, | Survival | 4-1BBL/IL-12 vs. 3 | *** | 0.0005 | 6C |
| Logrank test | | ng/kg rIL-12 | | | |
| (Bonferroni, | | | | | |
| k = 5) | | | | | |
| Kaplan Meier, | Survival | 4-1BBL/IL-12 vs. 500 | *** | 0.001 | 6C |
| Logrank test | | ng/kg rIL-12 | | | |
| (Bonferroni, | | | | | |
| k = 5) | | | | | |
| Kaplan Meier, | Survival | 4-1BBL/IL-12 vs. fLuc | *** | 0.0005 | 6C |
| Logrank test | | | | | |
| (Bonferroni, | | | | | |
| k = 5) | | | | | |
| Kaplan Meier, | Survival | 4-1BBL/IL-12 vs. 4G₅12 | ns | >0.9999 | 6C |
| Logrank test | | | | | |
| (Bonferroni, | | | | | |
| k = 5) | | | | | |
| Kaplan Meier, | Survival | 4-1BBL/IL-12 vs. 4T12 | ns | >0.9999 | 6C |
| Logrank test | | | | | |
| (Bonferroni, | | | | | |
| k = 5) | | | | | |
| Two-way | Tumor Area: | t = 11 | ns | 0.845 | 9A |
| ANOVA | Rechallenge | Naive vs. 4-1BBL/IL-12 | | | |
| (Dunnett post | | | | | |
| hoc test) | | | | | |
| Two-way | Tumor Area: | t = 11 | ns | 0.8822 | 9A |
| ANOVA | Rechallenge | Naive vs. 4G₅12 | | | |
| (Dunnett post | | | | | |
| hoc test) | | | | | |
| Two-way | Tumor Area: | t = 11 | ns | 0.8822 | 9A |
| ANOVA | Rechallenge | Naive vs. 4T12 | | | |
| (Dunnett post | | | | | |
| hoc test) | | | | | |
| Two-way | Tumor Area: | t = 13 | ns | 0.6636 | 9A |
| ANOVA | Rechallenge | Naive vs. 4-1BBL/IL-12 | | | |
| (Dunnett post | | | | | |
| hoc test) | | | | | |
| Two-way | Tumor Area: | t = 13 | ns | 0.7322 | 9A |
| ANOVA | Rechallenge | Naive vs. 4G512 | | | |
| (Dunnett post | | | | | |
| hoc test) | | | | | |
| Two-way | Tumor Area: | t = 13 | ns | 0.7322 | 9A |
| ANOVA | Rechallenge | Naive vs. 4T12 | | | |
| (Dunnett post | | | | | |
| hoc test) | | | | | |
| Two-way | Tumor Area: | t = 15 | ns | 0.1796 | 9A |
| ANOVA | Rechallenge | Naive vs. 4-1BBL/IL-12 | | | |
| (Dunnett post | | | | | |
| hoc test) | | | | | |
| Two-way | Tumor Area: | t = 15 | ns | 0.2552 | 9A |
| ANOVA | Rechallenge | Naive vs. 4G512 | | | |
| (Dunnett post | | | | | |
| hoc test) | | | | | |
| Two-way | Tumor Area: | t = 15 | ns | 0.2552 | 9A |
| ANOVA | Rechallenge | Naive vs. 4T12 | | | |
| (Dunnett post | | | | | |
| hoc test) | | | | | |
| Two-way | Tumor Area: | t = 17 | ns | 0.297 | 9A |
| ANOVA | Rechallenge | Naive vs. 4-1BBL/IL-12 | | | |
| (Dunnett post | | | | | |
| hoc test) | | | | | |
| Two-way | Tumor Area: | t = 17 | ns | 0.3847 | 9A |
| ANOVA | Rechallenge | Naive vs. 4G512 | | | |
| (Dunnett post | | | | | |
| hoc test) | | | | | |
| Two-way | Tumor Area: | t = 17 | ns | 0.3847 | 9A |
| ANOVA | Rechallenge | Naive vs. 4T12 | | | |
| (Dunnett post | | | | | |
| hoc test) | | | | | |
| Two-way | Tumor Area: | t = 19 | * | 0.0447 | 9A |
| ANOVA | Rechallenge | Naive vs. 4-1BBL/IL-12 | | | |
| (Dunnett post | | | | | |
| hoc test) | | | | | |
| Two-way | Tumor Area: | t = 19 | ns | 0.0808 | 9A |
| ANOVA | Rechallenge | Naive vs. 4G512 | | | |
| (Dunnett post | | | | | |
| hoc test) | | | | | |
| Two-way | Tumor Area: | t = 19 | ns | 0.0808 | 9A |
| ANOVA | Rechallenge | Naive vs. 4T12 | | | |
| (Dunnett post | | | | | |
| hoc test) | | | | | |
| Two-way | Tumor Area: | t = 21 | ** | 0.0014 | 9A |
| ANOVA | Rechallenge | Naive vs. 4-1BBL/IL-12 | | | |
| (Dunnett post | | | | | |
| hoc test) | | | | | |
| Two-way | Tumor Area: | t = 21 | ** | 0.0045 | 9A |
| ANOVA | Rechallenge | Naive vs. 4G512 | | | |
| (Dunnett post | | | | | |
| hoc test) | | | | | |
| Two-way | Tumor Area: | t = 21 | ** | 0.0045 | 9A |
| ANOVA | Rechallenge | Naive vs. 4T12 | | | |
| (Dunnett post | | | | | |
| hoc test) | | | | | |
| One-way | Transfection | pUNO GFP vs. Nanoplasmid GFP | **** | <0.0001 | 10A |
| ANOVA | (% GFP) | | | | |
| (Dunnett post | | | | | |
| hoc test) | | | | | |
| One-way | Transfection | pUNO GFP vs. Untreated | **** | <0.0001 | 10A |
| ANOVA | (% GFP) | | | | |
| (Dunnett post | | | | | |
| hoc test) | | | | | |
| One-way | GFP MFI | pUNO GFP vs. Nanoplasmid GFP | ** | 0.0063 | 10B |
| ANOVA | | | | | |
| (Dunnett post | | | | | |
| hoc test) | | | | | |
| One-way | GFP MFI | pUNO GFP vs. Untreated | * | 0.0108 | 1B |
| ANOVA | | | | | |
| (Dunnett post | | | | | |
| hoc test) | | | | | |
| Two-way | Tumor | t = 9 | ns | 0.9911 | 10C |
| ANOVA | Growth | fLuc vs pUNO 4-1BBL/IL-12 | | | |
| (Dunnett post | | | | | |
| hoc test) | | | | | |
| Two-way | Tumor | t = 9 | ns | 0.6423 | 10C |
| ANOVA | Growth | fLuc vs Nanoplasmid 4-1BBL/IL-12 | | | |
| (Dunnett post | | | | | |
| hoc test) | | | | | |
| Two-way | Tumor | t = 11 | ns | 0.9792 | 10C |
| ANOVA | Growth | fLuc vs pUNO 4-1BBL/IL-12 | | | |
| (Dunnett post | | | | | |
| hoc test) | | | | | |
| Two-way | Tumor | t = 11 | ns | 0.4661 | 10C |
| ANOVA | Growth | fLuc vs Nanoplasmid 4-1BBL/IL-12 | | | |
| (Dunnett post | | | | | |
| hoc test) | | | | | |
| Two-way | Tumor | t = 13 | ns | 0.2959 | 10C |
| ANOVA | Growth | fLuc vs pUNO 4-1BBL/IL-12 | | | |
| (Dunnett post | | | | | |
| hoc test) | | | | | |
| Two-way | Tumor | t = 13 | ns | 0.9987 | 10C |
| ANOVA | Growth | fLuc vs Nanoplasmid 4-1BBL/IL-12 | | | |
| (Dunnett post | | | | | |
| hoc test) | | | | | |
| Two-way | Tumor | t = 15 | ** | 0.0051 | 10C |
| ANOVA | Growth | fLuc vs pUNO 4-1BBL/IL-12 | | | |
| (Dunnett post | | | | | |
| hoc test) | | | | | |
| Two-way | Tumor | t = 15 | ns | 0.0762 | 10C |
| ANOVA | Growth | fLuc vs Nanoplasmid 4-1BBL/IL-12 | | | |
| (Dunnett post | | | | | |
| hoc test) | | | | | |
| Two-way | Tumor | t = 17 | **** | <0.0001 | 10C |
| ANOVA | Growth | fLuc vs pUNO 4-1BBL/IL-12 | | | |
| (Dunnett post | | | | | |
| hoc test) | | | | | |
| Two-way | Tumor | t = 17 | **** | <0.0001 | 10C |
| ANOVA | Growth | fLuc vs Nanoplasmid 4-1BBL/IL-12 | | | |
| (Dunnett post | | | | | |
| hoc test) | | | | | |
| Two-way | Tumor | t = 9 | ns | 0.7767 | 10L |
| ANOVA | Growth | fLuc + aPD1 vs pUNO 4- | | | |
| (Dunnett post | | 1BBL/IL-12 + aPD1 | | | |
| hoc test) | | | | | |
| Two-way | Tumor | t = 9 | ns | 0.972 | 10L |
| ANOVA | Growth | fLuc + aPD1 vs Nanoplasmid | | | |
| (Dunnett post | | 4-1BBL/IL-12 + aPD1 | | | |
| hoc test) | | | | | |
| Two-way | Tumor | t = 11 | ns | 0.4766 | 10D |
| ANOVA | Growth | fLuc + aPD1 vs pUNO 4- | | | |
| (Dunnett post | | 1BBL/IL-12 + aPD1 | | | |
| hoc test) | | | | | |
| Two-way | Tumor | t = 11 | ns | 0.8949 | 10D |
| ANOVA | Growth | fLuc + aPD1 vs Nanoplasmid | | | |
| (Dunnett post | | 4-1BBL/IL-12 + aPD1 | | | |
| hoc test) | | | | | |
| Two-way | Tumor | t = 13 | ns | 0.8156 | 10D |
| ANOVA | Growth | fLuc + aPD1 vs pUNO 4- | | | |
| (Dunnett post | | 1BBL/IL-12 + aPD1 | | | |
| hoc test) | | | | | |
| Two-way | Tumor | t = 13 | ns | 0.8531 | 10D |
| ANOVA | Growth | fLuc + aPD1 vs Nanoplasmid | | | |
| (Dunnett post | | 4-1BBL/IL-12 + aPD1 | | | |
| hoc test) | | | | | |
| Two-way | Tumor | t = 15 | ns | 0.0641 | 10D |
| ANOVA | Growth | fLuc + aPD1 vs pUNO 4- | | | |
| (Dunnett post | | 1BBL/IL-12 + aPD1 | | | |
| hoc test) | | | | | |
| Two-way | Tumor | t = 15 | ns | 0.11 | 10D |
| ANOVA | Growth | fLuc + aPD1 vs Nanoplasmid | | | |
| (Dunnett post | | 4-1BBL/IL-12 + aPD1 | | | |
| hoc test) | | | | | |
| Two-way | Tumor | t = 17 | **** | <0.0001 | 10D |
| ANOVA | Growth | fLuc + aPD1 vs pUNO 4- | | | |
| (Dunnett post | | 1BBL/IL-12 + aPD1 | | | |
| hoc test) | | | | | |
| Two-way | Tumor | t = 17 | **** | <0.0001 | 10D |
| ANOVA | Growth | fLuc + aPD1 vs Nanoplasmid | | | |
| (Dunnett post | | 4-1BBL/IL-12 + aPD1 | | | |
| hoc test) | | | | | |
| Kaplan Meier, | Survival | fLuc vs pUNO 4-1BBL/IL-12 | 0.0022 | ** | 10E |
| Logrank test | | | | | |
| (Bonferroni, | | | | | |
| k = 2) | | | | | |
| Kaplan Meier, | Survival | fLuc vs Nanoplasmid | 0.0002 | *** | 10E |
| Logrank test | | 4-1BBL/IL-12 | | | |
| (Bonferroni, | | | | | |
| k = 2) | | | | | |
| Kaplan Meier, | Survival | fLuc + aPD1 vs pUNO 4- | 0.0158 | * | 10F |
| Logrank test | | 1BBL/IL-12 + aPD1 | | | |
| (Bonferroni, | | | | | |
| k = 2) | | | | | |
| Kaplan Meier, | Survival | fLuc + aPD1 vs Nanoplasmid 4- | 0.0004 | *** | 10F |
| Logrank test | | 1BBL/IL-12 + aPD1 | | | |
| (Bonferroni, | | | | | |
| k = 2) | | | | | |

Flow cytometry staining for IL-12 revealed that 23±9% of cells that received 4G512 expressed IL-12 on the cell surface (FIG. 6E), indicating that the naturally secreted cytokine was successfully tethered to 4-1BBL and expressed on the extracellular side of the cell.

ELISA measurement detected no significant differences in secreted IL-12 when the cytokine was delivered on a separate plasmid (18±3 μg/mL) compared to delivery of 4T12 (8±2 μg/mL) and 12T4 (12±5 μg/mL) (FIG. 6F). However, IL-12 concentration was significantly lower (0.030±0.004 μg/mL) upon delivery of 4G512. Paired with the IL-12 staining on flow cytometry, this result indicated that IL-12 was successfully tethered to 4-1BBL in the context of this fusion protein.

### Example 6

During the development of embodiments of the technology described herein, experiments were conducted to show that tAPC delivery to cancer cells induces robust immune cell activation in vitro. Upon confirming the expression of 4-1BBL and IL-12 in the bicistronic plasmids, experiments were conducted to assess if T-cell activation was impacted by delivery of various plasmid formats. B16-F10 melanoma cells were treated with NPs delivering 4-1BBL and IL-12 as separate plasmids, 4G512, 4T12, 12T4, or GFP, and then co-cultured with C57BL/6J splenocytes (FIG. 6G). IFNγ secretion was quantified as a measure of immune stimulation typical of an anti-tumor response. IFNγ expression was significantly increased when the plasmids were delivered separately, (1300±200 pg/mL) compared to delivery of 4G512 (80±20 pg/mL), 4T12 (220±40 pg/mL), or 12T4 (50±10 pg/mL) (FIG. 6H).

Next, the effects of modifying linker length were investigated using the 4-1BBL (G4S)n IL-12 construct. IFNγ levels were not significantly different between the different linker lengths, although they trended towards reduced levels and variation was greater for the longest linker length (n=7) construct (FIG. 6I). Hence, the 4G512 construct was used for subsequent studies.

### Example 7

During the development of embodiments of the technology described herein, experiments were conducted to show that NP delivery of bicistronic 4-1BBL and IL-12 plasmids extends survival in B16-F10 tumor model. To determine whether in vitro immune cell activation by tAPCs would provide anti-tumor activity in animal models, C57BL/6J mice were inoculated with B16-F10 melanoma cells (t=0) and then received intratumoral injections (t=7, 9, 11 days) of 5-3-6 PBAE NPs delivering luciferase (as an experimental control), 4-1BBL and IL-12 (as separate plasmids, denoted 4-1BBL/IL-12), 4G512, 4T12, or (FIG. 6A). Additionally, recombinant IL-12 (rIL-12) (t=7, 11, 14, 18, 21, 25 days) were administered systemically because this therapy has been previously evaluated in a Phase 1 clinical treatment in various cancer types including melanoma. Two rIL-12 treatment cohorts were included and they were dosed at either the previously reported maximum tolerated dose (MTD) of IL-12 (500 ng/kg) or a significantly lower dose (3 ng/kg).

Mice that received a systemic injection of rIL-12 or luciferase NPs demonstrated faster tumor growth compared to those that received gene delivered 4-1BBL and IL-12 in any of the plasmid formats (FIG. 6B). While there was no significant difference in tumor growth between mice receiving 4-1BBL/IL-12 NPs and either 4G512 NPs or 4T12 NPs, significantly faster tumor growth was observed in mice treated with either dose of systemic rIL-12 or luciferase NPs. Similarly, mice receiving 3 ng/kg of IL-12 and 500 ng/kg rIL-12 both exhibited a median survival of 21 days, and mice receiving luciferase NPs also exhibited an average survival of 21 days (FIG. 6C). Moreover, there were no long-term survivors in the rIL-12- or luciferase NP-treated groups. Mice that received 4-1BBL/IL-12 (median survival of 46 days and 3 long term survivors), 4T12 (median survival of 53 days and 2 long term survivors), and 4G512 NPs (median survival of 31 days and 2 long term survivors) showed considerably better outcomes. Furthermore, no significant difference in survival was observed between mice that received 4-1BBL/IL-12 NPs and mice that received 4G512 NPs or 4T12 NPs. Individual mouse tumor plots demonstrated a delay in tumor growth for mice treated with gene-delivered 4-1BBL and IL. 12 compared to the rIL-12 and luciferase NP cohorts (FIG. 6D to FIG. 6I).

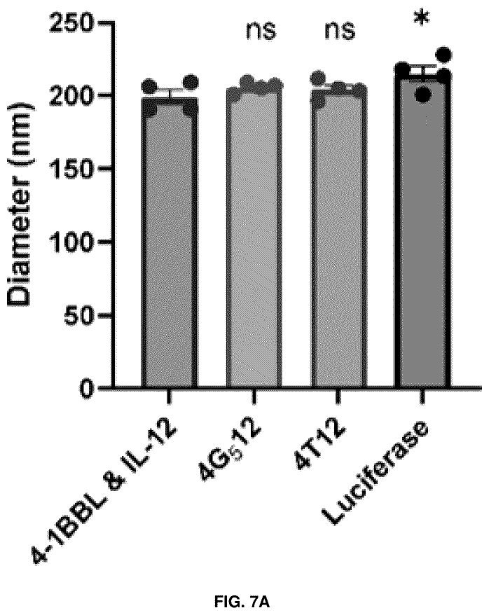

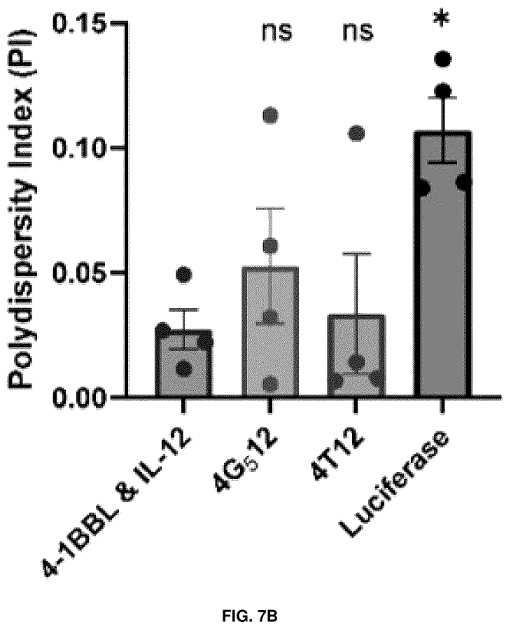

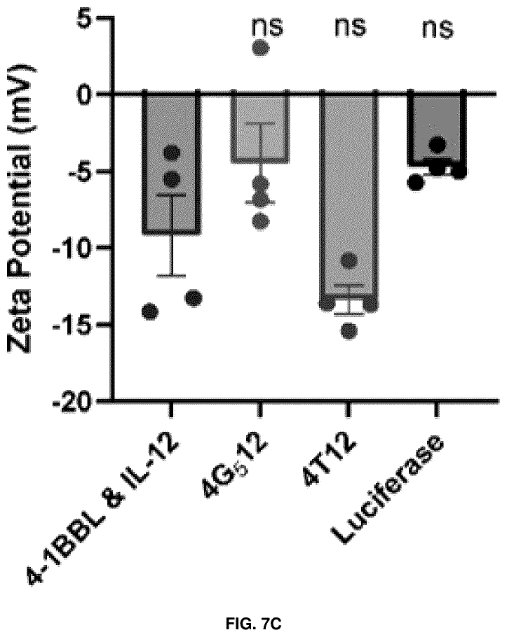

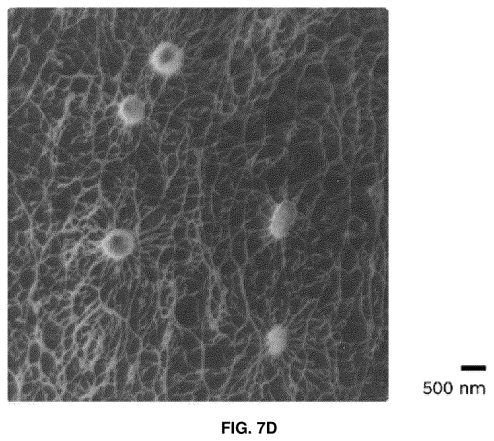

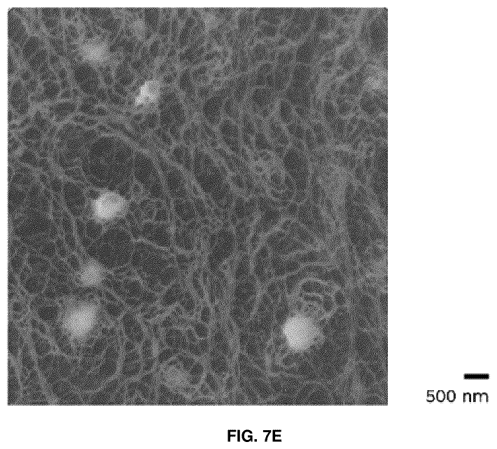

Dynamic light scattering (DLS) analysis revealed that PBAE NPs were in the size range of 200 nm in hydrodynamic diameter, with slightly negative zeta potentials. Nanoparticle diameters were measured for co-delivered 4-1BBL/IL-12 NPs (199±5 nm), 4G512 NPs (205±2 nm), 4T12 NPs (204±3 nm), and luciferase NPs (215±6 nm) (FIG. 7A). The polydispersity indices were all below 0.15, indicating a narrow size distribution (FIG. 7B). Zeta potentials were also measured for co-delivered 4-1BBL/IL-12 NPs (−9±3 mV), 4G512 (−4.5±3 mV), 4T12 (−13±1 mV), and luciferase (−5±1 mV) (FIG. 7C). No statistically significant differences were detected among the 4-1BBL/IL-12 NP sizes, polydispersity indices, or zeta potentials compared to either of the bicistronic plasmids.

Transmission electron microscopy (TEM) was used to visualize dried NPs formed with separated 4-1BBL and IL-12 plasmids (FIG. 7D) and NPs formed with the bicistronic 4T12 plasmid (FIG. 7E). Spherical shapes without aggregation were observed, confirming the successful formation of distinct NPs.

### Example 8

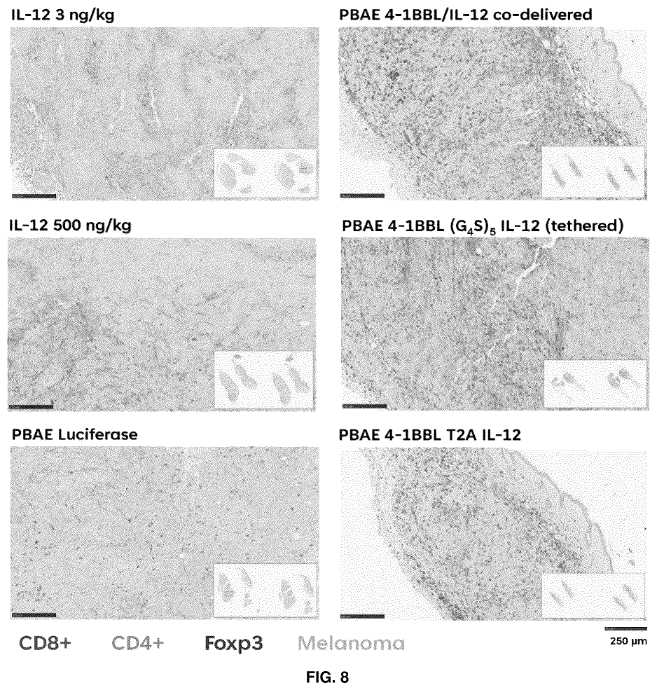

During the development of embodiments of the technology described herein, experiments were conducted to show that NP delivery of bicistronic 4-1BBL and IL-12 plasmids leads to CD4+ and CD8+ T cell infiltration into tumor. Tumors were harvested one day after the last injection of rIL-12 or NPs and stained for CD8+ (purple), CD4+ (teal), and FOXP3+ (dark brown) T cells. Melanoma was visualized as the beige/light brown regions due to melanin deposits (FIG. 8). Tumors in mice that had received rIL-12 or luciferase NPs exhibit little to no T-cell infiltration. Tumors in mice that received 4-1BBL/IL-12 NPs demonstrated high levels of CD4+ and CD8+ T cell infiltration into the tumor. Tumors treated with 4G512 NPs showed similar CD4+ T cell infiltration compared to the 4-1BBL/IL-12 group, although fewer CD8+ T cells were observed. Tumors in the 4T12 group had similar levels of CD4+ expression and CD8+ expression as those in the 4-1BBL/IL-12 group.

### Example 9

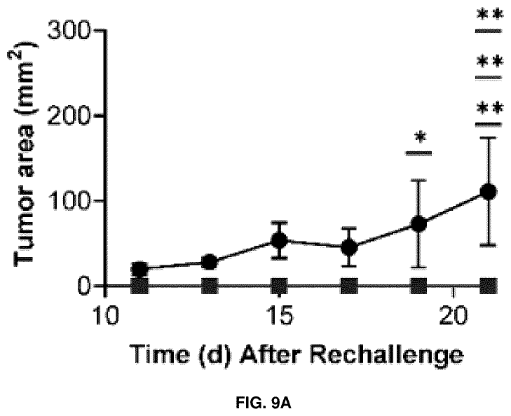

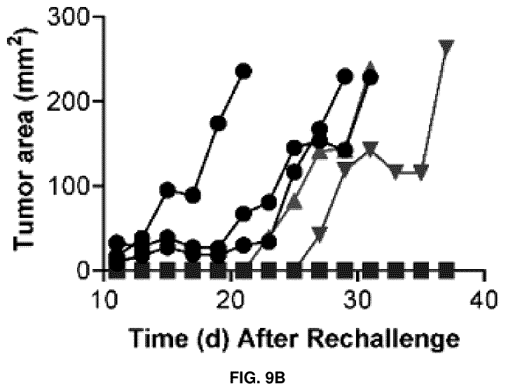

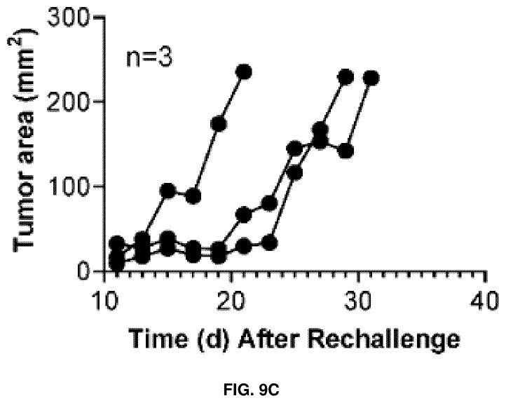

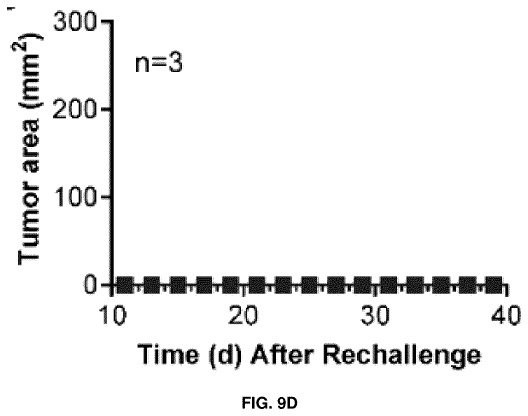

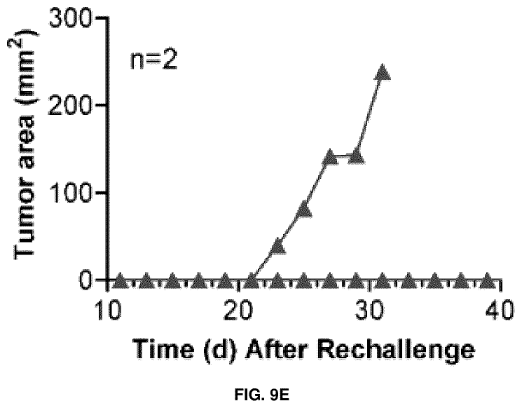

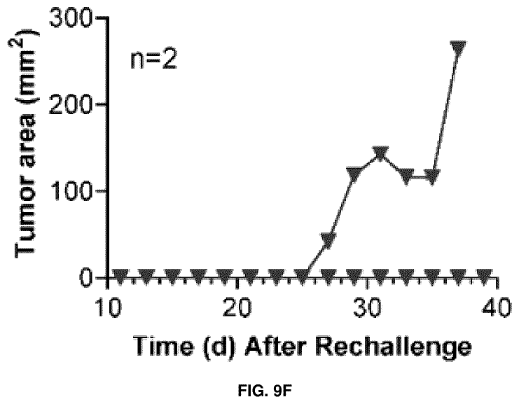

During the development of embodiments of the technology described herein, experiments were conducted to show that tAPC NP delivery elicits immune memory. One hundred (100) days after the initial tumor implantation, seven long-term survivors and three age-matched naïve mice were inoculated with B16-F10 tumors on the opposite flank. Three long-term survivors belonged to the group treated with 4-1BBL/IL-12, two to the group treated with 4G512, and two to the group treated with 4T12. All naïve mice developed tumors with a median survival of 25 days. Tumor growth in naïve mice was also significantly faster than in mice that had been previously treated (p<0.0001) (FIG. 9A). In groups that were previously treated, if a new tumor was formed, it grew later than in the previously untreated naïve mice (FIG. 9B). All three naive mice developed tumors, no mice treated with 4-1BBL/IL-12 NPs developed tumors, and one of the two mice developed a tumor in the groups treated with 4G512 or 4T12 (FIG. 9D to 9F).

### Example 10

During the development of embodiments of the technology described herein, experiments were conducted to show that translationally relevant antibiotic resistance gene-free Nanoplasmids demonstrate similar efficacy to pUNO1 plasmids. To ensure compliance with GMP production, Nanoplasmids from Aldeveron were used to create an antibiotic resistance gene-free version of the 4T12 bicistronic plasmid, a minimal plasmid without extra bacterial elements. GFP versions of the Nanoplasmid and PUNO1 plasmids were delivered to B16-F10 cells to compare expression. NPs were formulated with 600 ng of plasmid and 60 w/w of 4-4-6 PBAE in 20 μL. Nanoplasmid NPs demonstrated 70±2% transfection efficiency, while pUNO1 NPs demonstrated 85±1% efficiency (FIG. 5A). However, MFI with Nanoplasmid NPs was more than 2-fold greater than with pUNO1 NPs (FIG. 5B).

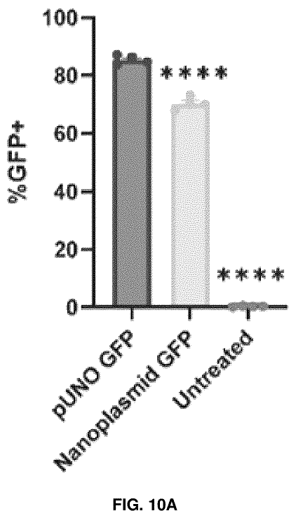

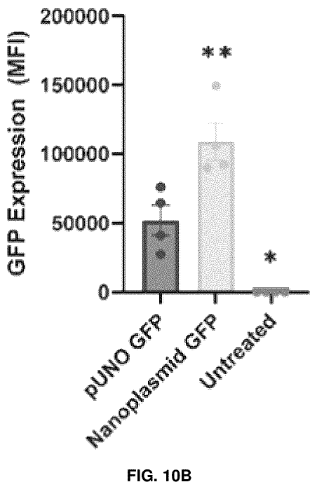

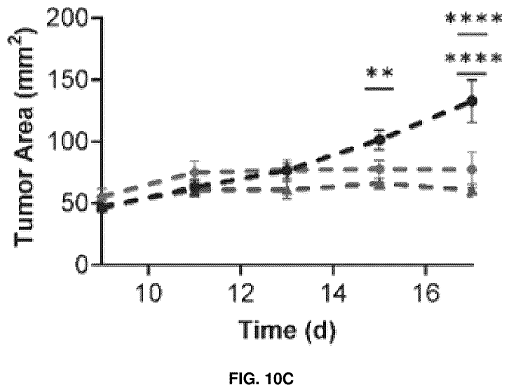

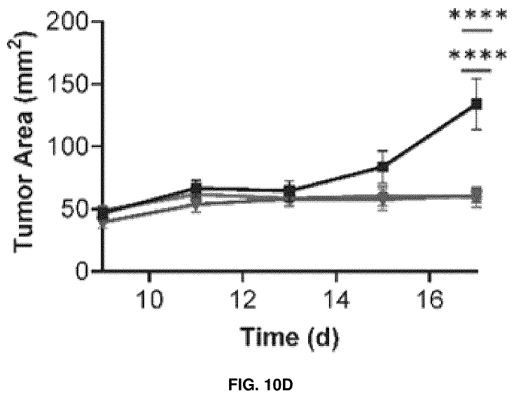

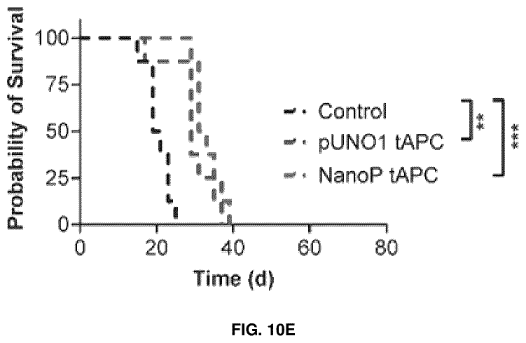

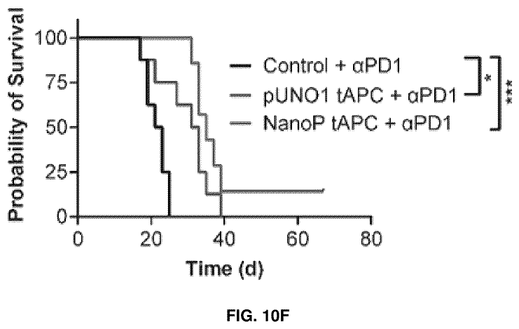

C57BL/6J mice were inoculated with B16-F10 melanoma cells on t=0, treated intratumorally (t=9, 11, 16, 18) with 5-3-6 PBAE NPs encoding co-delivered 4-1BBL/IL-12 using either the pUNO1 or Nanoplasmid backbone vectors. Tumors in mice that were treated with luciferase NPs grew faster than those treated with tAPC NPs, regardless of plasmid used (FIG. 10C and FIG. 10D). Mice that were treated with luciferase NPs demonstrated a median survival time of 20 days, which increased to 22 days with the additional administration of anti-PD1 (FIG. 10E and FIG. 10F). Mice treated with tAPC NPs formed with pUNO1 plasmids demonstrated a median survival time of 29 days, increasing to 32 days with anti-PD1, and mice treated with tAPC NPs formed with Nanoplasmids demonstrated a median survival time of 32 days, increasing to 35 days with addition of anti-PD1. Only the group treated with Nanoplasmid tAPC NPs and anti-PD1 had a long-term survivor.

### Example 11

Additional experiments were conducted to characterize bicistronic plasmids and fusion proteins for use in tumor immune reprogramming.

### Engineering Bicistronic Plasmids for Co-Expression of 4-1BBL and IL-12

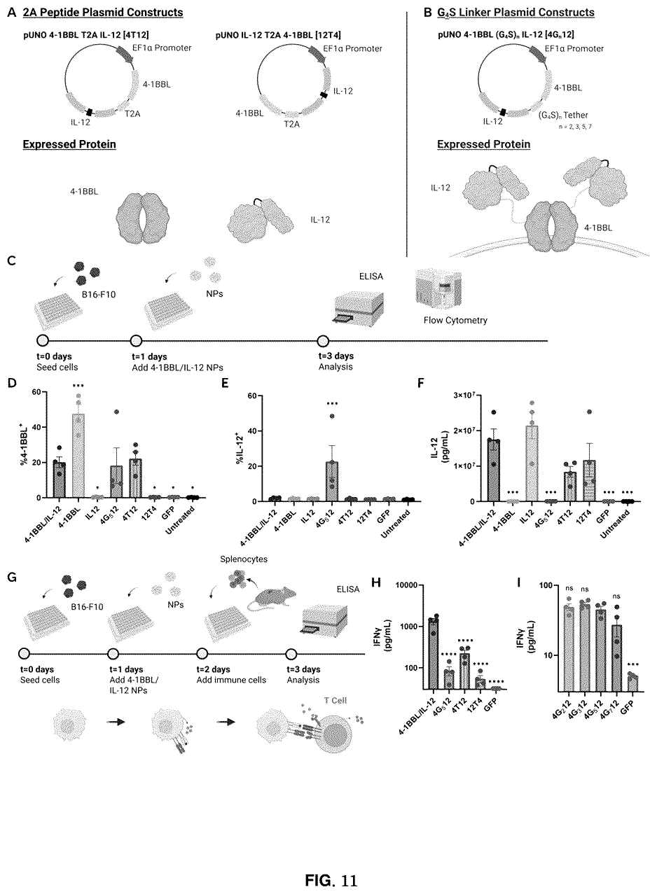

Six plasmid constructs containing 4-1BBL and IL-12 were generated, two of which incorporated the 2A peptide sequence, and four with varying lengths of the (G₄S)ₙ peptide tether (FIGS. 11A and B; Table 1). T2A, derived from thosea asigna virus, was chosen as the 2A sequence since it has the highest cleavage efficiency among the 2A peptides, thus ensuring that the proteins would not remain linked. In plasmids with the T2A sequence, one was formed with 4-1BBL at the N-terminus (4T12), and the other was formed with IL-12 at the N-terminus (12T4) to explore if sequence order affected expression and efficacy of tumor reprogramming. In the plasmids encoding the fusion protein with the (G₄S)ₙ tether, four lengths of the (G₄S)ₙ repeat (n=2, 3, 5, 7), corresponding to 10, 15, 25, and 35 amino acids were tested to evaluate which tether length resulted in the most effective tumor reprogramming. These constructs were labeled as 4Gn12 and designed so the (G₄S)ₙ repeat is positioned between the C terminus of 4-1BBL and the N terminus of IL-12 in order to enforce extracellular expression of IL-12, as 4-1BBL is a Type 2 transmembrane protein with a C-terminal extracellular domain. All plasmids were cloned into the pUNO1 backbone.

### Optimized PBAE NP Formulation Enhances In Vitro Transfection

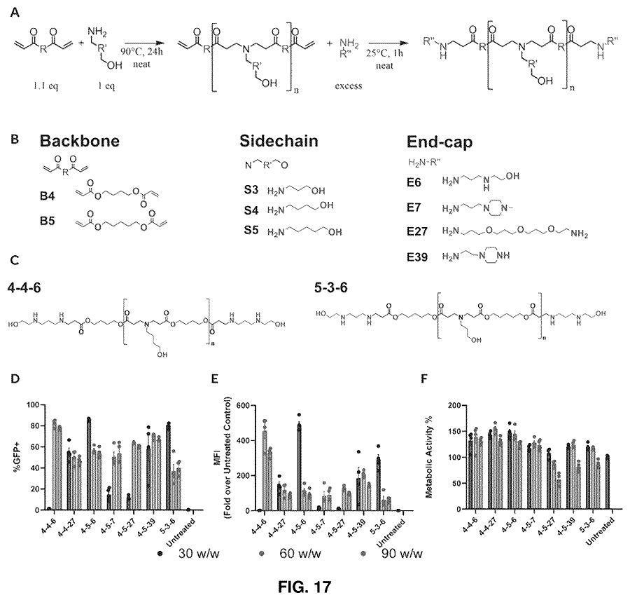

B16-F10 melanoma cells were selected to test the transfection efficiency of the engineered bicistronic plasmids since the tAPC platform has previously been validated in this model. GFP plasmid delivery via seven PBAE NPs was evaluated in order to select a formulation that enabled robust transfection and would facilitate downstream analysis of bicistronic plasmid protein expression. PBAE synthesis and structures can be found in FIG. 17A-C. Each PBAE was evaluated at 30, 60, and 90 w/w (weight/weight polymer mass to DNA mass ratio) with 600 ng of DNA. The five formulations that achieved the highest transfection efficacy were PBAE 4-5-6 at 30 w/w (85 (1) %), PBAE 4-4-6 at 60 w/w (82 (2) %), PBAE 5-3-6 at 30 w/w (79 (1) %), PBAE 4-4-6 at 90 w/w (78 (1) %), and PBAE Apr. 5, 1939 at 60 w/w (70 (1) %) (FIG. 17D). PBAE 4-4-6, also known as 2-(3-Aminopropylamino) ethanol end-terminated poly(1,4-butanediol diacrylate-co-4-amino-1-butanol), constituted two of the five formulations with the highest transfection and was thus chosen for further in vitro testing. The 60 w/w formulation was selected due to higher GFP geometric mean fluorescence intensity over untreated cells (~460-fold) (FIG. 17E) and higher cellular viability (FIG. 17F) compared to the 90 w/w formulation.

### Bicistronic Plasmids Enable Co-Expression of 4-1BBL and IL-12 in Multiple Formats

The plasmids were assessed in vitro to compare 4-1BBL and IL-12 expression when the two immunomodulatory signals were delivered on a single plasmid or on separate plasmids (FIG. 11C). Flow cytometry analysis of transfected cells showed that 20 (6) % of cells were 4-1BBL⁺ when 4-1BBL and IL-12 were delivered on separate plasmids (4-1BBL/IL-12) (FIG. 11D). In the 4-1BBL⁺ only group, expression of 4-1BBL⁺ was significantly higher, as approximately twice the mass of 4-1BBL plasmid was delivered than in the group with 4-1BBL/IL-12. Notably, when compared to co-delivery of 4-1BBL and IL-12 individual plasmids, the percentage of 4-1BBL⁺ cells was not significantly different following transfection with 4G₅12 (18 (10) %) or 4T12 (22 (4) %). However, the percentage of 4-1BBL⁺ cells was significantly lower in the 12T4 condition (0.2 (0.1) %) compared to co-delivery of the two plasmids (detailed statistical analysis is provided in Table 4). These results indicate that 4-1BBL can be delivered in a bicistronic format without negatively impacting levels of protein expression.

Flow cytometry staining for IL-12 revealed that 23 (9) % of cells that received 4G₅12 expressed IL-12 on the cell surface (FIG. 11E). Although IL-12 is naturally a secreted cytokine, these results indicate that IL-12 was successfully tethered, thus not secreted, to the c-terminus of 4-1BBL via the GS linker. Additionally, this demonstrates the presence of IL-12 on the extracellular side of the cell.

ELISA measurement detected no significant differences in secreted IL-12 when the cytokine was delivered on a separate plasmid (18 (3) μg/mL) compared to delivery of 4T12 (8 (2) μg/mL) and 12T4 (12 (5) μg/mL) (FIG. 11F). On the other hand, IL-12 concentration in the supernatant was significantly lower (0.030 (0.004) μg/mL) upon delivery of 4G₅12. Paired with the detection of cell-surface IL-12 via flow cytometry, this result confirmed that IL-12 was successfully tethered to 4-1BBL in the context of this fusion protein.

Fluorescence microscopy further verified the presence of IL-12 only on the cell surface in B16-F10 cells that were transfected with 4G₅12.

TABLE 4

| | | | | Adjusted | |
|---|---|---|---|---|---|
| Statistical test | Measurement | Comparison | Summary | P Value | Figure |
| One-way | %4-1BBL⁺ | 4-1BBL/IL-12 vs. 4-1BBL | *** | 0.001 | 11D |
| ANOVA | Expression | | | | |
| (Dunnett post | | | | | |
| hoc test) | | | | | |
| One-way | %4-1BBL⁺ | 4-1BBL/IL-12 vs. IL12 | * | 0.0214 | 11D |
| ANOVA | Expression | | | | |
| (Dunnett post | | | | | |
| hoc test) | | | | | |
| One-way | %4-1BBL⁺ | 4-1BBL/IL-12 vs. 4G₅12 | ns | 0.9998 | 11D |
| ANOVA | Expression | | | | |
| (Dunnett post | | | | | |
| hoc test) | | | | | |
| One-way | %4-1BBL⁺ | 4-1BBL/IL-12 vs. 4T12 | ns | 0.9996 | 11D |
| ANOVA | Expression | | | | |
| (Dunnett post | | | | | |
| hoc test) | | | | | |
| One-way | %4-1BBL⁺ | 4-1BBL/IL-12 vs. 12T4 | * | 0.0208 | 11D |
| ANOVA | Expression | | | | |
| (Dunnett post | | | | | |
| hoc test) | | | | | |
| One-way | %4-1BBL⁺ | 4-1BBL/IL-12 vs. GFP | * | 0.0214 | 11D |
| ANOVA | Expression | | | | |
| (Dunnett post | | | | | |
| hoc test) | | | | | |
| One-way | %4-1BBL⁺ | 4-1BBL/IL-12 vs. Untreated | * | 0.02 | 11D |
| ANOVA | Expression | | | | |
| (Dunnett post | | | | | |
| hoc test) | | | | | |
| One-way | % IL-12⁺ | 4-1BBL/IL-12 vs. 4-1BBL | ns | >0.9999 | 11E |
| ANOVA | Expression | | | | |
| (Dunnett post | | | | | |
| hoc test) | | | | | |
| One-way | % IL-12⁺ | 4-1BBL/IL-12 vs. IL12 | ns | >0.9999 | 11E |
| ANOVA | Expression | | | | |
| (Dunnett post | | | | | |
| hoc test) | | | | | |
| One-way | % IL-12⁺ | 4-1BBL/IL-12 vs. 4G512 | *** | 0.0008 | 11E |
| ANOVA | Expression | | | | |
| (Dunnett post | | | | | |
| hoc test) | | | | | |
| One-way | % IL-12⁺ | 4-1BBL/IL-12 vs. 4T12 | ns | >0.9999 | 11E |
| ANOVA | Expression | | | | |
| (Dunnett post | | | | | |
| hoc test) | | | | | |
| One-way | % IL-12⁺ | 4-1BBL/IL-12 vs. 12T4 | ns | >0.9999 | 11E |
| ANOVA | Expression | | | | |
| (Dunnett post | | | | | |
| hoc test) | | | | | |
| One-way | % IL-12⁺ | 4-1BBL/IL-12 vs. GFP | ns | >0.9999 | 11E |
| ANOVA | Expression | | | | |
| (Dunnett post | | | | | |
| hoc test) | | | | | |
| One-way | % IL-12⁺ | 4-1BBL/IL-12 vs. Untreated | ns | >0.9999 | 11E |
| ANOVA | Expression | | | | |
| (Dunnett post | | | | | |
| hoc test) | | | | | |
| One-way | IL-12 Concentration | 4-1BBL/IL-12 vs. 4-1BBL | *** | 0.0002 | 11F |
| ANOVA | | | | | |
| (Dunnett post | | | | | |
| hoc test) | | | | | |
| One-way | IL-12 Concentration | 4-1BBL/IL-12 vs. IL12 | ns | 0.7648 | 11F |
| ANOVA | | | | | |
| (Dunnett post | | | | | |
| hoc test) | | | | | |
| One-way | IL-12 Concentration | 4-1BBL/IL-12 vs. 4G512 | *** | 0.0002 | 11F |
| ANOVA | | | | | |
| (Dunnett post | | | | | |
| hoc test) | | | | | |
| One-way | IL-12 Concentration | 4-1BBL/IL-12 vs. 4T12 | ns | 0.074 | 11F |
| ANOVA | | | | | |
| (Dunnett post | | | | | |
| hoc test) | | | | | |
| One-way | IL-12 Concentration | 4-1BBL/IL-12 vs. 12T4 | ns | 0.4154 | 11F |
| ANOVA | | | | | |
| (Dunnett post | | | | | |
| hoc test) | | | | | |
| One-way | IL-12 Concentration | 4-1BBL/IL-12 vs. GFP | *** | 0.0002 | 11F |
| ANOVA | | | | | |
| (Dunnett post | | | | | |
| hoc test) | | | | | |
| One-way | IL-12 Concentration | 4-1BBL/IL-12 vs. Untreated | *** | 0.0002 | 11F |
| ANOVA | | | | | |
| (Dunnett post | | | | | |
| hoc test) | | | | | |
| One-way | IFNγ Concentration | 4-1BBL/IL-12 vs. 4G512 | **** | <0.0001 | 11H |
| ANOVA | | | | | |
| (Dunnett post | | | | | |
| hoc test) | | | | | |
| One-way | IFNγ Concentration | 4-1BBL/IL-12 vs. 4T12 | **** | <0.0001 | 11H |
| ANOVA | | | | | |
| (Dunnett post | | | | | |
| hoc test) | | | | | |
| One-way | IFNγ Concentration | 4-1BBL/IL-12 vs. 12T4 | **** | <0.0001 | 11H |
| ANOVA | | | | | |
| (Dunnett post | | | | | |
| hoc test) | | | | | |
| One-way | IFNγ Concentration | 4-1BBL/IL-12 vs. GFP | **** | <0.0001 | 11H |
| ANOVA | | | | | |
| (Dunnett post | | | | | |
| hoc test) | | | | | |
| One-way | IFNγ Concentration | 4G512 vs. GFP | *** | 0.0004 | 11I |
| ANOVA | | | | | |
| (Dunnett post | | | | | |
| hoc test) | | | | | |
| One-way | IFNγ Concentration | 4G512 vs. 4G212 | ns | 0.9704 | 11I |
| ANOVA | | | | | |
| (Dunnett post | | | | | |
| hoc test) | | | | | |
| One-way | IFNγ Concentration | 4G512 vs. 4G312 | ns | 0.6529 | 11I |
| ANOVA | | | | | |
| (Dunnett post | | | | | |
| hoc test) | | | | | |
| One-way | IFNy Concentration | 4G512 vs. 4G712 | ns | 0.1085 | 11I |
| ANOVA | | | | | |
| (Dunnett post | | | | | |
| hoc test) | | | | | |
| Two-way | Tumor Area | t = 7 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 7 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 9 | ns | 0.9853 | 12B |
| ANOVA | | Signal 2 + Signal 3 vs. fLuc | | | |
| (Dunnett post | | | | | |
| hoc test) | | | | | |
| Two-way | Tumor Area | t = 9 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 9 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 9 | ns | 0.8192 | 12B |
| ANOVA | | Signal 2 + Signal 3 vs. fLuc | | | |
| (Dunnett post | | | | | |
| hoc test) | | | | | |
| Two-way | Tumor Area | t = 9 | ns | 0.971 | 12B |
| ANOVA | | Signal 2 + Signal 3 vs. fLuc | | | |
| (Dunnett post | | | | | |
| hoc test) | | | | | |
| Two-way | Tumor Area | t = 11 | ns | 0.2341 | 12B |
| ANOVA | | Signal 2 + Signal 3 vs. fLuc | | | |
| (Dunnett post | | | | | |
| hoc test) | | | | | |
| Two-way | Tumor Area | t = 11 | ns | 0.992 | 12B |
| ANOVA | | Signal 2 + Signal 3 vs. fLuc | | | |
| (Dunnett post | | | | | |
| hoc test) | | | | | |
| Two-way | Tumor Area | t = 11 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 11 | *** | 0.0001 | 12B |
| ANOVA | | Signal 2 + Signal 3 vs. fLuc | | | |
| (Dunnett post | | | | | |
| hoc test) | | | | | |
| Two-way | Tumor Area | t = 11 | ns | 0.1833 | 12B |
| ANOVA | | Signal 2 + Signal 3 vs. fLuc | | | |
| (Dunnett post | | | | | |
| hoc test) | | | | | |
| Two-way | Tumor Area | t = 13 | *** | 0.0009 | 12B |
| ANOVA | | Signal 2 + Signal 3 vs. fLuc | | | |
| (Dunnett post | | | | | |
| hoc test) | | | | | |
| Two-way | Tumor Area | t = 13 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 13 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 13 | **** | <0.0001 | 12B |
| ANOVA | | Signal 2 + Signal 3 vs. fLuc | | | |
| (Dunnett post | | | | | |
| hoc test) | | | | | |
| Two-way | Tumor Area | t = 13 | *** | 0.0009 | 12B |
| ANOVA | | Signal 2 + Signal 3 vs. fLuc | | | |
| (Dunnett post | | | | | |
| hoc test) | | | | | |
| Two-way | Tumor Area | t = 15 | **** | <0.0001 | 12B |
| ANOVA | | Signal 2 + Signal 3 vs. fLuc | | | |
| (Dunnett post | | | | | |
| hoc test) | | | | | |
| Two-way | Tumor Area | t = 15 | ns | >0.9999 | 12B |
| ANOVA | | Signal 2 + Signal 3 vs. fLuc | | | |
| (Dunnett post | | | | | |
| hoc test) | | | | | |
| Two-way | Tumor Area | t = 15 | ns | 0.8476 | 12B |
| ANOVA | | Signal 2 + Signal 3 vs. fLuc | | | |
| (Dunnett post | | | | | |
| hoc test) | | | | | |
| Two-way | Tumor Area | t = 15 | **** | <0.0001 | 12B |
| ANOVA | | Signal 2 + Signal 3 vs. fLuc | | | |
| (Dunnett post | | | | | |
| hoc test) | | | | | |
| Two-way | Tumor Area | t = 15 | **** | <0.0001 | 12B |
| ANOVA | | Signal 2 + Signal 3 vs. fLuc | | | |
| (Dunnett post | | | | | |
| hoc test) | | | | | |
| Kaplan Meier, | Survival | 4-1BBL/IL-12 vs. 3 ng/kg | *** | 0.0005 | 12C |
| Logrank test | | rIL-12 | | | |
| (Bonferroni, | | | | | |
| k = 5) | | | | | |
| Kaplan Meier, | Survival | 4-1BBL/IL-12 vs. 500 ng/kg | *** | 0.001 | 12C |
| Logrank test | | rIL-12 | | | |
| (Bonferroni, | | | | | |
| k = 5) | | | | | |
| Kaplan Meier, | Survival | 4-1BBL/IL-12 vs. fLuc | *** | 0.0005 | 12C |
| Logrank test | | | | | |
| (Bonferroni, | | | | | |
| k = 5) | | | | | |
| Kaplan Meier, | Survival | 4-1BBL/IL-12 vs. 4G₅12 | ns | >0.9999 | 12C |
| Logrank test | | | | | |
| (Bonferroni, | | | | | |
| k = 5) | | | | | |
| Kaplan Meier, | Survival | 4-1BBL/IL-12 vs. 4T12 | ns | >0.9999 | 12C |
| Logrank test | | | | | |
| (Bonferroni, | | | | | |
| k = 5) | | | | | |
| Two-way | Tumor Area: | t = 11 | ns | 0.845 | 14A |
| ANOVA | Rechallenge | Naive vs. 4-1BBL/IL-12 | | | |
| (Dunnett post | | | | | |
| hoc test) | | | | | |
| Two-way | Tumor Area: | t = 11 | ns | 0.8822 | 14A |
| ANOVA | Rechallenge | Naive vs. 4G₅12 | | | |
| (Dunnett post | | | | | |
| hoc test) | | | | | |
| Two-way | Tumor Area: | t = 11 | ns | 0.8822 | 14A |
| ANOVA | Rechallenge | Naive vs. 4T12 | | | |
| (Dunnett post | | | | | |
| hoc test) | | | | | |
| Two-way | Tumor Area: | t = 13 | ns | 0.6636 | 14A |
| ANOVA | Rechallenge | Naive vs. 4-1BBL/IL-12 | | | |
| (Dunnett post | | | | | |
| hoc test) | | | | | |
| Two-way | Tumor Area: | t = 13 | ns | 0.7322 | 14A |
| ANOVA | Rechallenge | Naive vs. 4G512 | | | |
| (Dunnett post | | | | | |
| hoc test) | | | | | |
| Two-way | Tumor Area: | t = 13 | ns | 0.7322 | 14A |
| ANOVA | Rechallenge | Naive vs. 4T12 | | | |
| (Dunnett post | | | | | |
| hoc test) | | | | | |
| Two-way | Tumor Area: | t = 15 | ns | 0.1796 | 14A |
| ANOVA | Rechallenge | Naive vs. 4-1BBL/IL-12 | | | |
| (Dunnett post | | | | | |
| hoc test) | | | | | |
| Two-way | Tumor Area: | t = 15 | ns | 0.2552 | 14A |
| ANOVA | Rechallenge | Naive vs. 4G512 | | | |
| (Dunnett post | | | | | |
| hoc test) | | | | | |
| Two-way | Tumor Area: | t = 15 | ns | 0.2552 | 14A |
| ANOVA | Rechallenge | Naive vs. 4T12 | | | |
| (Dunnett post | | | | | |
| hoc test) | | | | | |
| Two-way | Tumor Area: | t = 17 | ns | 0.297 | 14A |
| ANOVA | Rechallenge | Naive vs. 4-1BBL/IL-12 | | | |
| (Dunnett post | | | | | |
| hoc test) | | | | | |
| Two-way | Tumor Area: | t = 17 | ns | 0.3847 | 14A |
| ANOVA | Rechallenge | Naive vs. 4G512 | | | |
| (Dunnett post | | | | | |
| hoc test) | | | | | |
| Two-way | Tumor Area: | t = 17 | ns | 0.3847 | 14A |
| ANOVA | Rechallenge | Naive vs. 4T12 | | | |
| (Dunnett post | | | | | |
| hoc test) | | | | | |
| Two-way | Tumor Area: | t = 19 | * | 0.0447 | 14A |
| ANOVA | Rechallenge | Naive vs. 4-1BBL/IL-12 | | | |
| (Dunnett post | | | | | |
| hoc test) | | | | | |
| Two-way | Tumor Area: | t = 19 | ns | 0.0808 | 14A |
| ANOVA | Rechallenge | Naive vs. 4G512 | | | |
| (Dunnett post | | | | | |
| hoc test) | | | | | |
| Two-way | Tumor Area: | t = 19 | ns | 0.0808 | 14A |
| ANOVA | Rechallenge | Naive vs. 4T12 | | | |
| (Dunnett post | | | | | |
| hoc test) | | | | | |
| Two-way | Tumor Area: | t = 21 | ** | 0.0014 | 14A |
| ANOVA | Rechallenge | Naive vs. 4-1BBL/IL-12 | | | |
| (Dunnett post | | | | | |
| hoc test) | | | | | |
| Two-way | Tumor Area: | t = 21 | ** | 0.0045 | 14A |
| ANOVA | Rechallenge | Naive vs. 4G512 | | | |
| (Dunnett post | | | | | |
| hoc test) | | | | | |
| Two-way | Tumor Area: | t = 21 | ** | 0.0045 | 14A |
| ANOVA | Rechallenge | Naive vs. 4T12 | | | |
| (Dunnett post | | | | | |
| hoc test) | | | | | |
| One-way | Transfection (% GFP) | pUNO GFP vs. Nanoplasmid | **** | <0.0001 | 15A |
| ANOVA | | GFP | | | |
| (Dunnett post | | | | | |
| hoc test) | | | | | |
| One-way | Transfection (% GFP) | PUNO GFP vs. Untreated | **** | <0.0001 | 15A |
| ANOVA | | | | | |
| (Dunnett post | | | | | |
| hoc test) | | | | | |
| One-way | GFP MFI | pUNO GFP vs. Nanoplasmid | ** | 0.0063 | 15B |
| ANOVA | | GFP | | | |
| (Dunnett post | | | | | |
| hoc test) | | | | | |
| One-way | GFP MFI | PUNO GFP vs. Untreated | * | 0.0108 | 15B |
| ANOVA | | | | | |
| (Dunnett post | | | | | |
| hoc test) | | | | | |
| Kaplan Meier, | Survival | Nanoplasmid fLuc + aPD1 | * | 0.0358 | 15C |
| Logrank test | | vs Nanoplasmid 4-1BBL/IL- | | | |
| (Bonferroni, | | 12 + aPD1 | | | |
| k = 1) | | | | | |
| One-way | Nanoparticle | 4-1BBL & IL-12 vs. | * | 0.0475 | 18A |
| ANOVA | Diameter | Luciferase | | | |
| (Dunnett post | | | | | |
| hoc test) | | | | | |
| One-way | Nanoparticle | 4-1BBL & IL-12 vs. 4T12 | ns | 0.7458 | 18A |
| ANOVA | Diameter | | | | |
| (Dunnett post | | | | | |
| hoc test) | | | | | |
| One-way | Nanoparticle | 4-1BBL & IL-12 vs. 4G512 | ns | 0.5886 | 18A |
| ANOVA | Diameter | | | | |
| (Dunnett post | | | | | |
| hoc test) | | | | | |
| One-way | Nanoparticle PDI | 4-1BBL & IL-12 vs. | * | 0.0248 | 18B |
| ANOVA | | Luciferase | | | |
| (Dunnett post | | | | | |
| hoc test) | | | | | |
| One-way | Nanoparticle PDI | 4-1BBL & IL-12 vs. 4T12 | ns | 0.9899 | 18B |
| ANOVA | | | | | |
| (Dunnett post | | | | | |
| hoc test) | | | | | |
| One-way | Nanoparticle PDI | 4-1BBL & IL-12 vs. 4G512 | ns | 0.6515 | 18B |
| ANOVA | | | | | |
| (Dunnett post | | | | | |
| hoc test) | | | | | |
| One-way | Nanoparticle Zeta | 4-1BBL & IL-12 vs. | ns | 0.2746 | 18C |
| ANOVA | Potential | Luciferase | | | |
| (Dunnett post | | | | | |
| hoc test) | | | | | |
| One-way | Nanoparticle Zeta | 4-1BBL & IL-12 vs. 4T12 | ns | 0.323 | 18C |
| ANOVA | Potential | | | | |
| (Dunnett post | | | | | |
| hoc test) | | | | | |
| One-way | Nanoparticle Zeta | 4-1BBL & IL-12 vs. 4G512 | ns | 0.241 | 18C |
| ANOVA | Potential | | | | |
| (Dunnett post | | | | | |
| hoc test) | | | | | |
| Two-way | Tumor Growth | t = 9 | ns | 0.9911 | 19A |
| ANOVA | | fLuc vs pUNO 4-1BBL/IL-12 | | | |
| (Dunnett post | | | | | |
| hoc test) | | | | | |
| Two-way | Tumor Growth | t = 9 | ns | 0.6423 | 19A |
| ANOVA | | fLuc vs Nanoplasmid 4- | | | |
| (Dunnett post | | 1BBL/IL-12 | | | |
| hoc test) | | | | | |
| Two-way | Tumor Growth | t = 11 | ns | 0.9792 | 19A |
| ANOVA | | fLuc vs pUNO 4-1BBL/IL-12 | | | |
| (Dunnett post | | | | | |
| hoc test) | | | | | |
| Two-way | Tumor Growth | t = 11 | ns | 0.4661 | 19A |
| ANOVA | | fLuc vs Nanoplasmid 4- | | | |
| (Dunnett post | | 1BBL/IL-12 | | | |
| hoc test) | | | | | |
| Two-way | Tumor Growth | t = 13 | ns | 0.2959 | 19A |
| ANOVA | | fLuc vs pUNO 4-1BBL/IL-12 | | | |
| (Dunnett post | | | | | |
| hoc test) | | | | | |
| Two-way | Tumor Growth | t = 13 | ns | 0.9987 | 19A |
| ANOVA | | fLuc vs Nanoplasmid 4- | | | |
| (Dunnett post | | 1BBL/IL-12 | | | |
| hoc test) | | | | | |
| Two-way | Tumor Growth | t = 15 | ** | 0.0051 | 19A |
| ANOVA | | fLuc vs pUNO 4-1BBL/IL-12 | | | |
| (Dunnett post | | | | | |
| hoc test) | | | | | |
| Two-way | Tumor Growth | t = 15 | ns | 0.0762 | 19/ |
| ANOVA | | fLuc vs Nanoplasmid 4- | | | |
| (Dunnett post | | 1BBL/IL-12 | | | |
| hoc test) | | | | | |
| Two-way | Tumor Growth | t = 17 | **** | <0.0001 | 19A |
| ANOVA | | fLuc vs pUNO 4-1BBL/IL-12 | | | |
| (Dunnett post | | | | | |
| hoc test) | | | | | |
| Two-way | Tumor Growth | t = 17 | **** | <0.0001 | 19A |
| ANOVA | | fLuc vs Nanoplasmid 4- | | | |
| (Dunnett post | | 1BBL/IL-12 | | | |
| hoc test) | | | | | |
| Two-way | Tumor Growth | t = 9 | ns | 0.7767 | 19B |
| ANOVA | | fLuc + aPD1 vs pUNO 4- | | | |
| (Dunnett post | | 1BBL/IL-12 + aPD1 | | | |
| hoc test) | | | | | |
| Two-way | Tumor Growth | t = 9 | ns | 0.972 | 19B |
| ANOVA | | fLuc + aPD1 vs Nanoplasmid | | | |
| (Dunnett post | | 4-1BBL/IL-12 + aPD1 | | | |
| hoc test) | | | | | |
| Two-way | Tumor Growth | t = 11 | ns | 0.4766 | 19B |
| ANOVA | | fLuc + aPD1 vs pUNO | | | |
| (Dunnett post | | 4-1BBL/IL-12 + aPD1 | | | |
| hoc test) | | | | | |
| Two-way | Tumor Growth | t = 11 | ns | 0.8949 | 19B |
| ANOVA | | fLuc + aPD1 vs Nanoplasmid | | | |
| (Dunnett post | | 4-1BBL/IL-12 + aPD1 | | | |
| hoc test) | | | | | |
| Two-way | Tumor Growth | t = 13 | ns | 0.8156 | 19B |
| ANOVA | | fLuc + aPD1 vs pUNO | | | |
| (Dunnett post | | 4-1BBL/IL-12 + aPD1 | | | |
| hoc test) | | | | | |
| Two-way | Tumor Growth | t = 13 | ns | 0.8531 | 19B |
| ANOVA | | fLuc + aPD1 vs Nanoplasmid | | | |
| (Dunnett post | | 4-1BBL/IL-12 + aPD1 | | | |
| hoc test) | | | | | |
| Two-way | Tumor Growth | t = 15 | ns | 0.0641 | 19B |
| ANOVA | | fLuc + aPD1 vs pUNO | | | |
| (Dunnett post | | 4-1BBL/IL-12 + aPD1 | | | |
| hoc test) | | | | | |
| Two-way | Tumor Growth | t = 15 | ns | 0.11 | 19B |
| ANOVA | | fLuc + aPD1 vs Nanoplasmid | | | |
| (Dunnett post | | 4-1BBL/IL-12 + aPD1 | | | |
| hoc test) | | | | | |
| Two-way | Tumor Growth | t = 17 | **** | <0.0001 | 19B |
| ANOVA | | fLuc + aPD1 vs pUNO | | | |
| (Dunnett post | | 4-1BBL/IL-12 + aPD1 | | | |
| hoc test) | | | | | |
| Two-way | Tumor Growth | t = 17 | **** | <0.0001 | 19B |
| ANOVA | | fLuc + aPD1 vs Nanoplasmid | | | |
| (Dunnett post | | 4-1BBL/IL-12 + aPD1 | | | |
| hoc test) | | | | | |
| Kaplan Meier, | Survival | fLuc vs pUNO 4-1BBL/IL-12 | 0.0022 | ** | 19C |
| Logrank test | | | | | |
| (Bonferroni, | | | | | |
| k = 2) | | | | | |
| Kaplan Meier, | Survival | fLuc vs Nanoplasmid 4- | 0.0002 | *** | 19C |
| Logrank test | | 1BBL/IL-12 | | | |
| (Bonferroni, | | | | | |
| k = 2) | | | | | |
| Kaplan Meier, | Survival | fLuc + aPD1 vs pUNO 4- | 0.0158 | * | 19D |
| Logrank test | | 1BBL/IL-12 + aPD1 | | | |
| (Bonferroni, | | | | | |
| k = 2) | | | | | |
| Kaplan Meier, | Survival | fLuc + aPD1 vs | 0.0004 | *** | 19D |
| Logrank test | | Nanoplasmid 4-1BBL/IL- | | | |
| (Bonferroni, | | 12 + aPD1 | | | |
| k = 2) | | | | | |
| Two-way | Tumor Growth | t = 7 | ns | 0.9997 | 20A |
| ANOVA | | Nanoplasmid fLuc + | | | |
| (Sidak's post | | aPD1 vs Nanoplasmid 4- | | | |
| hoc test) | | 1BBL/IL-12 + aPD1 | | | |
| Two-way | Tumor Growth | t = 9 | ns | 0.9963 | 20A |
| ANOVA | | Nanoplasmid fLuc + | | | |
| (Sidak's post | | aPD1 vs Nanoplasmid 4- | | | |
| hoc test) | | 1BBL/IL-12 + aPD1 | | | |
| Two-way | Tumor Growth | t = 11 | ns | 0.6769 | 20A |
| ANOVA | | Nanoplasmid fLuc + | | | |
| (Sidak's post | | aPD1 vs Nanoplasmid 4- | | | |
| hoc test) | | 1BBL/IL-12 + aPD1 | | | |
| Two-way | Tumor Growth | t = 13 | ns | 0.8117 | 20A |
| ANOVA | | Nanoplasmid fLuc + | | | |
| (Sidak's post | | aPD1 vs Nanoplasmid 4- | | | |
| hoc test) | | 1BBL/IL-12 + aPD1 | | | |
| Two-way | Tumor Growth | t = 15 | * | 0.0105 | 20A |
| ANOVA | | Nanoplasmid fLuc + | | | |
| (Sidak's post | | aPD1 vs Nanoplasmid 4- | | | |
| hoc test) | | 1BBL/IL-12 + aPD1 | | | |
| Two-way | Tumor Growth | t = 17 | ** | 0.0041 | 20A |
| ANOVA | | Nanoplasmid fLuc + | | | |
| (Sidak's post | | aPD1 vs Nanoplasmid 4- | | | |
| hoc test) | | 1BBL/IL-12 + aPD1 | | | |

tAPC Delivery to Cancer Cells Induces Robust Immune Cell Activation In Vitro

After confirming the expression of 4-1BBL and IL-12 from the bicistronic plasmids, it was assessed whether T-cell activation was impacted by delivery of various plasmid formats. B16-F10 melanoma cells were treated with NPs delivering 4-1BBL and IL-12 as separate plasmids, 4G₅12, 4T12, 12T4, or GFP, and then co-cultured with C57BL/6J splenocytes (FIG. 11G). IFNγ secretion was quantified as a measure of immune stimulation typical of an anti-tumor response.

IFNγ expression was significantly higher when the plasmids were delivered separately (1300 (200) pg/mL) compared to delivery of 4G₅12 (80 (20) pg/mL), 4T12 (220 (40) pg/mL), or 12T4 (50 (10) pg/mL) (FIG. 11H).

The effects of modifying linker length in the 4-1BBL (G₄S)ₙ IL-12 construct was next investigated. Linker length represents the maximum distance the tethered IL-12 can be from the surface bound 4-1BBL and has little effect on the quantity of IL-12 present. If similar levels of IL-12 were expressed, then linker length would not be expected to affect IFNγ levels. However, if IL-12 were sterically hindered as a result of using shorter linkers, then IFNγ levels would be expected to be lower in groups with shorter linkers. Thus, multiple increasing linker lengths were tested. IFNγ levels were not significantly different among groups treated with constructs of different linker lengths although variability was greater for the construct with the longest linker length ((G₄S)₇) (FIG. 11I). Hence, the 4G₅12 construct was used for subsequent studies.

### NP Delivery of Bicistronic 4-1BBL and IL-12 Plasmids Extends Survival in B16-F10 Tumor Model

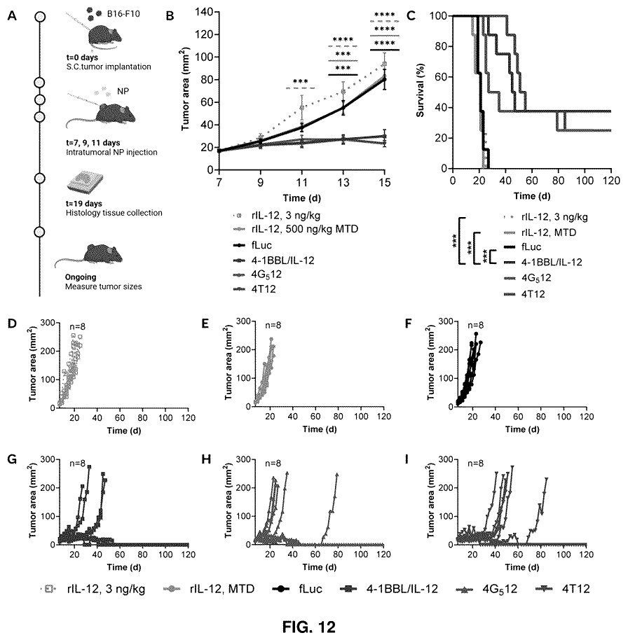

To determine whether in vitro immune cell activation by tAPCs would translate into anti-tumor activity in vivo, C57BL/6J mice were inoculated with B16-F10 melanoma cells (t=0) and received intratumoral injections (t=7, 9, 11 days) of 2-(3-Aminopropylamino) ethanol end-terminated poly(1,4-pentanediol diacrylate-co-3-amino-1propanol) PBAE NPs, or 5-3-6 PBAE NPs, delivering a control plasmid (luciferase), 4-1BBL and IL-12 (as separate plasmids, denoted 4-1BBL/IL-12), 4G₅12, or 4T12 (FIG. 12A). Additionally, intravenous recombinant IL-12 (rIL-12) (t=7, 11, 14, 18, 21, 25 days) was administered. Two rIL-12 treatment cohorts were included, dosed at either the previously reported maximum tolerated dose (MTD) of IL-12 in patients (500 ng/kg) or the lowest reported dose (3 ng/kg). All mice received 100 μg of systemic anti-PD1 (t=7, 9, 11 days), as anti-PD1 therapy is a gold-standard immunotherapy for metastatic melanoma currently used in the clinic. This was therefore used as a benchmark in the studies in order to show that combination treatment with the NPs is beneficial. By delivering 4-1BBL and IL-12 into tumor cells, T cell activation against the tumor cells was increased, and this is expected to synergize with anti-PD1, which blocks the inactivation of T cells.

Mice that received a systemic injection of rIL-12 or intratumoral luciferase NPs experienced significantly faster tumor growth compared to those that had received DNA nanoparticles carrying 4-1BBL and IL-12 in any of the plasmid formats (FIG. 12B). In comparison, there was no significant difference between mice receiving 4-1BBL/IL-12 NPs and either 4G₅12 NPs or 4T12 NPs. Similarly, mice receiving 3 ng/kg of IL-12 and 500 ng/kg rIL-12 both exhibited a median survival of 21 days, and mice receiving luciferase NPs also exhibited an average survival of 21 days (FIG. 12C). Moreover, there were no long-term survivors in the rIL-12- or luciferase NP-treated groups. Mice that received 4-1BBL/IL-12 (median survival of 46 days and 3 long term survivors), 4T12 (median survival of 53 days and 2 long term survivors), and 4G₅12 NPs (median survival of 31 days and 2 long term survivors) showed considerably better outcomes. Furthermore, no significant difference in survival was observed between mice that received 4-1BBL/IL-12 NPs and mice that received 4G₅12 NPs or 4T12 NPs (FIG. 12C). Individual mouse tumor plots highlighted the delay in tumor growth for mice treated with gene-delivered 4-1BBL and IL-12 compared to the rIL-12 and luciferase NP cohorts (FIG. 12D-I).

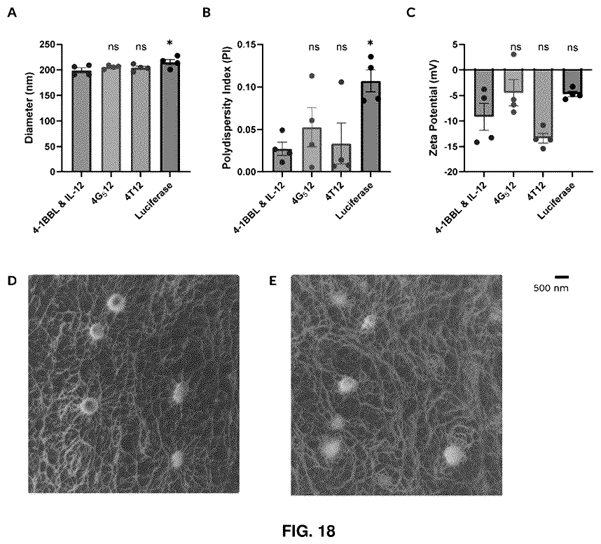

Dynamic light scattering (DLS) analysis revealed that PBAE NPs used for in vivo studies were in the size range of 200 nm in hydrodynamic diameter, with slightly negative zeta potentials. NP diameters were measured for co-delivered 4-1BBL/IL-12 NPs (199 (5) nm), 4G₅12 NPs (205 (2) nm), 4T12 NPs (204 (3) nm), and luciferase NPs (215 (6) nm) (FIG. 18A). The polydispersity indices were all below 0.15, indicating a narrow size distribution (FIG. 18B). Zeta potentials were also measured for co-delivered 4-1BBL/IL-12 NPs (−9 (3) mV), 4G₅12 (−4.5 (3) mV), 4T12 (−13 (1) mV), and luciferase (−5 (1) mV) (FIG. 18C). No statistically significant differences were detected among the 4-1BBL/IL-12 NP sizes, polydispersity indices, or zeta potentials compared to either of the bicistronic plasmids.

Transmission electron microscopy (TEM) was employed to visualize dried NPs formed with separate 4-1BBL and IL-12 plasmids (FIG. 18D) and NPs formed with the bicistronic 4T12 plasmid (FIG. 18E) that were used in vivo. Spherical shapes without aggregation were observed, confirming the successful formation of distinct NPs.

NP Delivery of Bicistronic 4-1BBL and IL-12 Plasmids Leads to CD4⁺ and CD8⁺ T-Cell Infiltration into Tumor

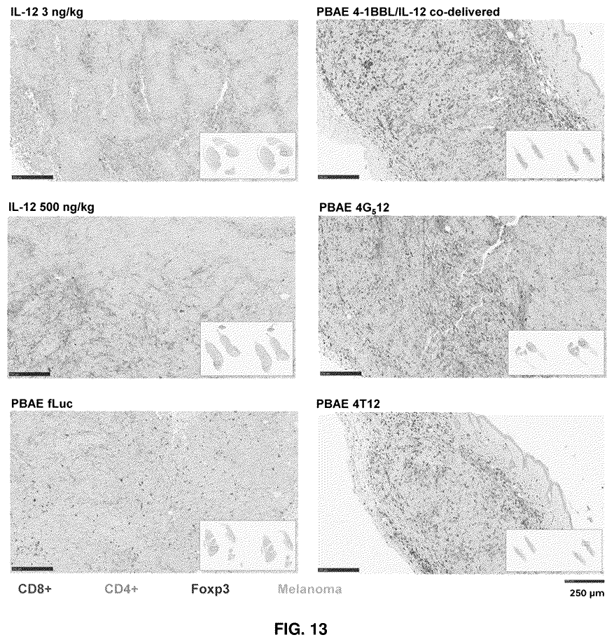

Tumors were excised one day after the last injection of rIL-12 or NPs and stained for CD8⁺, CD4⁺, and FOXP3⁺ T cells. Melanoma can be visualized as the beige/light brown regions due to melanin deposits (FIG. 13). Tumors in mice that had received rIL-12 or luciferase NPs exhibit little to no T-cell infiltration. Tumors in mice that received 4-1BBL/IL-12 NPs demonstrated high levels of CD4⁺ and CD8⁺ T-cell infiltration into the tumor. Tumors treated with 4G₅12 NPs showed similar CD4⁺ T-cell infiltration compared to the 4-1BBL/IL-12 group, although fewer CD8⁺ T cells were observed. This observation led to an investigation of whether tethering IL-12 to the cell surface might limit effective immune cell recruitment into solid tumors and may explain the reduced therapeutic efficacy compared to other plasmid formats. Tumors in the 4T12 group had similar levels of CD4⁺ and CD8⁺ infiltration compared to those in the 4-1BBL/IL-12 group. FOXP3⁺ Treg infiltration did not seem to vary between treatment groups, indicating that there was a lack of an anti-inflammatory response.

tAPC NP Delivery can Elicit Long-Lasting Anti-Tumor Immune Responses

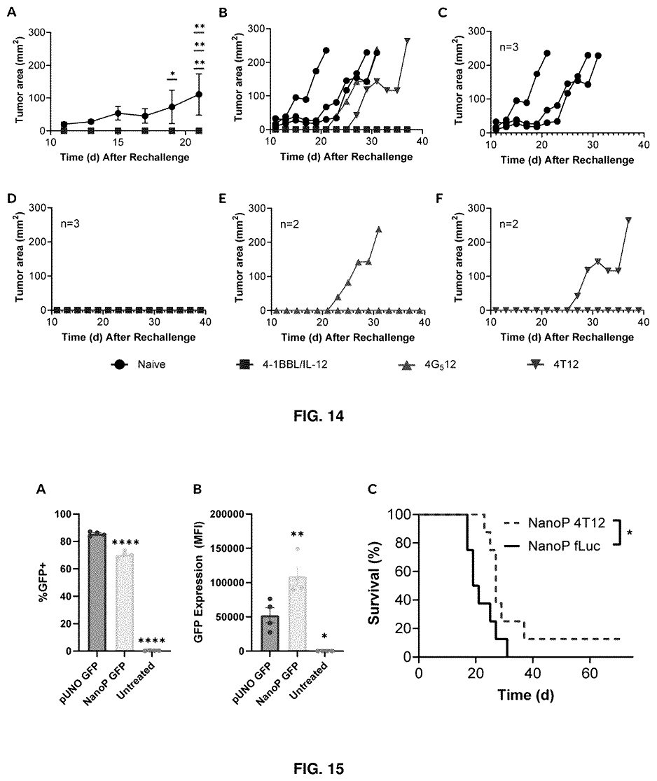

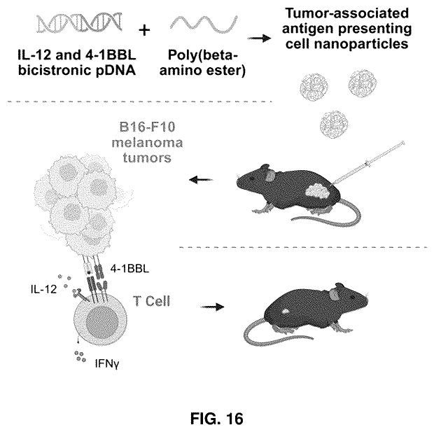

One hundred (100) days after the initial tumor implantation, seven long-term survivors and three age-matched naïve mice were inoculated with B16-F10 tumors on the opposite flank. Three long-term survivors belonged to the group treated with 4-1BBL/IL-12, two to the group treated with 4G₅12, and two to the group treated with 4T12. All naïve mice developed tumors with a median survival of 25 days. Tumor growth in naïve mice was also significantly faster than in mice that had been previously treated (p<0.0001) (FIG. 14A). In groups that had been previously treated, if a new tumor formed, it grew later than in the previously untreated naïve mice (FIG. 14B). All three naive mice developed tumors, no mice treated with 4-1BBL/IL-12 NPs developed tumors, and one of the two mice developed a tumor in the groups treated with 4G₅12 or 4T12 (FIG. 14D-F). In a prior study, mice previously treated with 4-1BBL/IL-12 NPs had higher B16-F10 antigen-specific T cells and delayed tumor growth. The resistance of new tumor growth in 4G₅12 or 4T12 NP treated mice demonstrates that the long-lasting anti-tumor immune response is not specific to the two-plasmid format.

Translationally Relevant Antibiotic Resistance Gene-Free Nanoplasmids (NanoP) Demonstrate Similar Efficacy to pUNO1 Plasmids

To ensure compliance with GMP production, Nanoplasmids from Aldevron were used to create an antibiotic resistance-free version of the 4T12 bicistronic plasmid, a minimal plasmid without extra bacterial elements. GFP versions of the Nanoplasmid and pUNO1 plasmids were delivered to B16-F10 cells to compare expression. NPs were formulated with 600 ng of plasmid and 60 w/w of 4-4-6 PBAE in 20 μL. NanoP NPs demonstrated 70 (2) % transfection efficiency, while pUNO1 NPs demonstrated 85 (1) % efficiency (FIG. 15A). However, GFP mean fluorescence intensity (MFI) with NanoP NPs was more than 2-fold greater than with pUNO1 NPs (FIG. 15B). Percent GFP is a binary measurement, representing whether cells expressed the delivered GFP gene, while MFI represents the strength of the GFP signal, a proxy measurement of the amount of protein produced. In this context, transfection with Nanoplasmid resulted in much stronger GFP signal in slightly fewer cells, which translates to stronger downstream expression of T cell activation signals 4-1BBL and IL-12. This may be due to the smaller size of the Nanoplasmid compared to the pUNO1 plasmid, allowing for higher copy number of the delivered gene by mass.

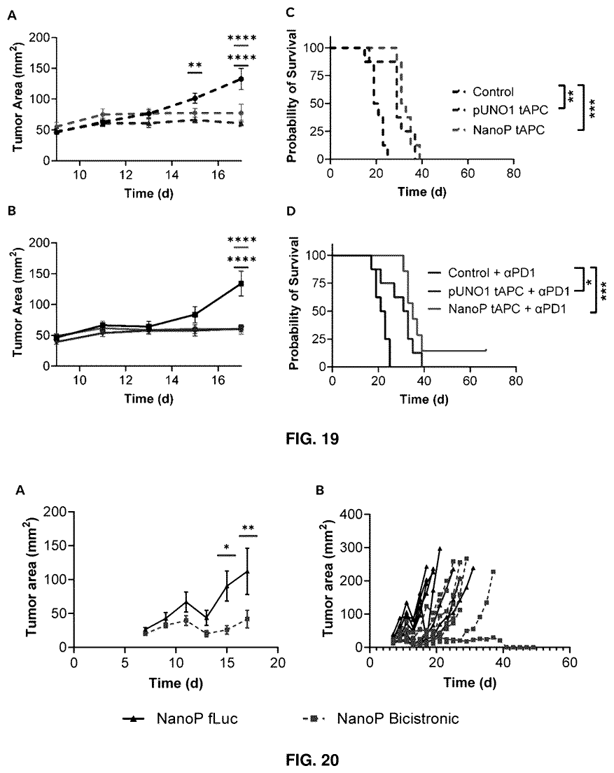

C57BL/6J mice were inoculated with B16-F10 melanoma cells on t=0 and treated intratumorally (t=9, 11, 16, 18) with 5-3-6 PBAE NPs co-delivering plasmids encoding 4-1BBL/IL-12 separately using either the pUNO1 or NanoP backbone vectors. Tumors that were treated with luciferase NPs grew faster than those treated with tAPC NPs, regardless of plasmid used (FIG. 19A-B). Mice that were treated with luciferase NPs demonstrated a median survival time of 20 days, which increased to 22 days with the additional administration of anti-PD1 (FIG. 19C-D). Mice treated with tAPC NPs formed with pUNO1 plasmids demonstrated a median survival time of 29 days, increasing to 32 days with anti-PD1, and mice treated with tAPC NPs formed with Nanoplasmids demonstrated a median survival time of 32 days, increasing to 35 days with addition of anti-PD1. Only the group treated with NanoP tAPC NPs and anti-PD1 had a long-term survivor.

In a further study, NanoP luciferase NPs were compared to NanoP bicistronic 4T12 NPs (FIG. 15C). The group treated with NanoP luciferase yielded no long-term survivors, while the group treated with 4T12 NPs yielded one long term survivor. Median survival was 20 days in the luciferase group and 27 days in the 4T12 group. Survival was significantly better in the 4T12 group (*p=0.0358). Average tumor growth and individual tumor growth can be found in FIG. 20A-B.

### REFERENCES

(1) Tzeng, S. Y.; Patel, K. K.; Wilson, D. R.; Meyer, R. A.; Rhodes, K. R.; Green, J. J. In Situ Genetic Engineering of Tumors for Long-Lasting and Systemic Immunotherapy. Proc Natl Acad Sci USA 2020, 117 (8), 4043-4052. doi.org/10.1073/pnas.1916039117.
(2) Luly, K. M.; Green, J. J.; Sunshine, J. C.; Tzeng, S. Y. Biomaterial-Mediated Genetic Reprogramming of Merkel Cell Carcinoma and Melanoma Leads to Targeted Cancer Cell Killing In Vitro and In Vivo. ACS Biomater Sci Eng 2023, 9 (11), 6438-6450. doi.org/10.1021/acsbiomaterials.3c00885.
(3) Neshat, S. Y.; Chan, C. H. R.; Harris, J.; Zmily, O. M.; Est-Witte, S.; Karlsson, J.; Shannon, S. R.; Jain, M.; Doloff, J. C.; Green, J. J.; Tzeng, S. Y. Polymeric Nanoparticle Gel for Intracellular mRNA Delivery and Immunological Reprogramming of Tumors. Biomaterials 2023, 300, 122185. doi.org/10.1016/j.biomaterials.2023.122185.
(4) Przybylowski, M.; Bartido, S.; Borquez Ojeda, O.; Sadelain, M.; Rivière, I. Production of Clinical-Grade Plasmid DNA for Human Phase I Clinical Trials and Large Animal Clinical Studies. Vaccine 2007, 25 (27), 5013-5024. doi.org/10.1016/j.vaccine.2007.04.077.
(5) Vandermeulen, G.; Marie, C.; Scherman, D.; Préat, V. New Generation of Plasmid Backbones Devoid of Antibiotic Resistance Marker for Gene Therapy Trials. Mol Ther 2011, 19 (11), 1942-1949. doi.org/10.1038/mt.2011.182.
(6) Liu, Z.; Chen, O.; Wall, J. B. J.; Zheng, M.; Zhou, Y.; Wang, L.; Ruth Vaseghi, H.; Qian, L.; Liu, J. Systematic Comparison of 2A Peptides for Cloning Multi-Genes in a Polycistronic Vector. Sci Rep 2017, 7 (1), 2193. doi.org/10.1038/s41598-017-02460-2.
(7) Klein, J. S.; Jiang, S.; Galimidi, R. P.; Keeffe, J. R.; Bjorkman, P. J. Design and Characterization of Structured Protein Linkers with Differing Flexibilities. Protein Eng Des Sel 2014, 27 (10), 325-330. doi.org/10.1093/protein/gzu043.
(8) Chen, X.; Zaro, J. L.; Shen, W. C. Fusion Protein Linkers: Property, Design and Functionality. Adv Drug Deliv Rev 2013, 65 (10), 1357-1369. doi.org/10.1016/j.addr.2012.09.039.
(9) Van Rosmalen, M.; Krom, M.; Merkx, M. Tuning the Flexibility of Glycine-Serine Linkers To Allow Rational Design of Multidomain Proteins. Biochemistry 2017, 56 (50), 6565-6574. doi.org/10.1021/acs.biochem.7b00902.
(10) Szymczak, A. L.; Workman, C. J.; Wang, Y.; Vignali, K. M.; Dilioglou, S.; Vanin, E. F.; Vignali, D. A. A. Correction of Multi-Gene Deficiency in Vivo Using a Single “self-Cleaving” 2A Peptide-Based Retroviral Vector. Nat Biotechnol 2004, 22 (5), 589-594. doi.org/10.1038/nbt957.
(11) Chng, J.; Wang, T.; Nian, R.; Lau, A.; Hoi, K. M.; Ho, S. C.; Gagnon, P.; Bi, X.; Yang, Y. Cleavage Efficient 2A Peptides for High Level Monoclonal Antibody Expression in CHO Cells. mAbs 2015, 7 (2), 403-412. doi.org/10.1080/19420862.2015.1008351.
(12) Fang, J.; Qian, J. J.; Yi, S.; Harding, T. C.; Tu, G. H.; VanRoey, M.; Jooss, K. Stable Antibody Expression at Therapeutic Levels Using the 2A Peptide. Nat Biotechnol 2005, 23 (5), 584-590. doi.org/10.1038/nbt1087.
(13) Chng, J.; Wang, T.; Nian, R.; Lau, A.; Hoi, K. M.; Ho, S. C.; Gagnon, P.; Bi, X.; Yang, Y. Cleavage Efficient 2A Peptides for High Level Monoclonal Antibody Expression in CHO Cells. MAbs 2015, 7 (2), 403-412. doi.org/10.1080/19420862.2015.1008351.
(14) Spahr, C.; Gunasekaran, K.; Walker, K. W.; Shi, S. D.-H. High-Resolution Mass Spectrometry Confirms the Presence of a Hydroxyproline (Hyp) Post-Translational Modification in the GGGGP Linker of an Fc-Fusion Protein. mAbs 2017, 9 (5), 812-819. doi.org/10.1080/19420862.2017.1325556.
(15) Spahr, C.; Shi, S. D.-H.; Lu, H. S. O-Glycosylation of Glycine-Serine Linkers in Recombinant Fc-Fusion Proteins: Attachment of Glycosaminoglycans and Other Intermediates with Phosphorylation at the Xylose Sugar Subunit. MAbs 2014, 6 (4), 904-914. doi.org/10.4161/mabs.28763.
(16) Arslan, M.; Karadag, M.; Onal, E.; Gelinci, E.; Cakan Akdogan, G.; Kalyoncu, S. Effect of Non-Repetitive Linker on in Vitro and in Vivo Properties of an Anti-VEGF scFv. Sci Rep 2022, 12 (1), 5449. doi.org/10.1038/s41598-022-09324-4.
(17) Mack, M.; Riethmüller, G.; Kufer, P. A Small Bispecific Antibody Construct Expressed as a Functional Single-Chain Molecule with High Tumor Cell Cytotoxicity. Proceedings of the National Academy of Sciences 1995, 92 (15), 7021-7025. doi.org/10.1073/pnas.92.15.7021.
(18) Arndt, M. A. E.; Krauss, J.; Vu, B. K.; Newton, D. L.; Rybak, S. M. A Dimeric Angiogenin Immunofusion Protein Mediates Selective Toxicity Toward CD22+ Tumor Cells. Journal of Immunotherapy 2005, 28 (3), 245. doi.org/10.1097/01.cji.0000161396.96582.10.
(19) Kjaergaard, M. Estimation of Effective Concentrations Enforced by Complex Linker Architectures from Conformational Ensembles. Biochemistry 2022, 61 (3), 171-182. doi.org/10.1021/acs.biochem. 1c00737.
(20) Mignon, C.; Sodoyer, R.; Werle, B. Antibiotic-Free Selection in Biotherapeutics: Now and Forever. Pathogens 2015, 4 (2), 157-181. doi.org/10.3390/pathogens4020157.
(21) Nanoplasmid FAQ. www.aldevron.com/custom-manufacturing/nanoplasmid/faq (accessed 2025 Apr. 25).
(22) Won, E.-Y.; Cha, K.; Byun, J.-S.; Kim, D.-U.; Shin, S.; Ahn, B.; Kim, Y. H.; Rice, A. J.; Walz, T.; Kwon, B. S.; Cho, H.-S. The Structure of the Trimer of Human 4-1BB Ligand Is Unique among Members of the Tumor Necrosis Factor Superfamily. Journal of Biological Chemistry 2010, 285 (12), 9202-9210. doi.org/10.1074/jbc.M109.084442.
(23) Gollob, J. A.; Mier, J. W.; Veenstra, K.; McDermott, D. F.; Clancy, D.; Clancy, M.; Atkins, M. B. Phase I Trial of Twice-Weekly Intravenous Interleukin 12 in Patients with Metastatic Renal Cell Cancer or Malignant Melanoma: Ability to Maintain IFN-Gamma Induction Is Associated with Clinical Response. Clin Cancer Res 2000, 6 (5), 1678-1692.
(24) Lasek, W.; Zagożdżon, R.; Jakobisiak, M. Interleukin 12: Still a Promising Candidate for Tumor Immunotherapy? Cancer Immunol Immunother 2014, 63 (5), 419-435. doi.org/10.1007/s00262-014-1523-1.
(25) Zugasti, I.; Espinosa Aroca, Lady; Fidyt, K.; Mulens-Arias, V.; Diaz-Beya, M.; Juan, M.; Urbano-Ispizua, A.; Esteve, J.; Velasco-Hernandez, T.; Menéndez, P. CAR-T Cell Therapy for Cancer: Current Challenges and Future Directions. Sig Transduct Target Ther 2025, 10 (1), 210. doi.org/10.1038/s41392-025-02269-w.
(26) Liu, J.; Cao, S.; Kim, S.; Chung, E. Y.; Homma, Y.; Guan, X.; Jimenez, V.; Ma, X. Interleukin-12: An Update on Its Immunological Activities, Signaling and Regulation of Gene Expression. Curr Immunol Rev 2005, 1 (2), 119-137. doi.org/10.2174/1573395054065115.
(27) Mazzolini, G.; Prieto, J.; Melero, I. Gene Therapy of Cancer with Interleukin-12. Curr Pharm Des 2003, 9 (24), 1981-1991. doi.org/10.2174/1381612033454261.
(28) Cheng, E. M.; Tsarovsky, N. W.; Sondel, P. M.; Rakhmilevich, A. L. Interleukin-12 as an in Situ Cancer Vaccine Component: A Review. Cancer Immunol Immunother 2022, 71 (9), 2057-2065. doi.org/10.1007/s00262-022-03144-1.
(29) Liu, J.-Q.; Zhang, C.; Zhang, X.; Yan, J.; Zeng, C.; Talebian, F.; Lynch, K.; Zhao, W.; Hou, X.; Du, S.; Kang, D. D.; Deng, B.; McComb, D. W.; Bai, X. F.; Dong, Y. Intratumoral Delivery of IL-12 and IL-27 mRNA Using Lipid Nanoparticles for Cancer Immunotherapy. Journal of Controlled Release 2022, 345, 306-313. doi.org/10.1016/j.jconrel.2022.03.021.
(30) Hu, J.; Yang, Q.; Zhang, W.; Du, H.; Chen, Y.; Zhao, Q.; Dao, L.; Xia, X.; Natalie Wall, F.; Zhang, Z.; Mahadeo, K.; Gorlick, R.; Kopetz, S.; Dotti, G.; Li, S. Cell Membrane-Anchored and Tumor-Targeted IL-12 (attIL12)-T Cell Therapy for Eliminating Large and Heterogeneous Solid Tumors. J Immunother Cancer 2022, 10 (1), e003633. doi.org/10.1136/jitc-2021-003633.
(31) Wang, C.; Li, J.; Chen, J.; Wang, Z.; Zhu, G.; Song, L.; Wu, J.; Li, C.; Qiu, R.; Chen, X.; Zhang, L.; Li, W. Multi-Omics Analyses Reveal Biological and Clinical Insights in Recurrent Stage I Non-Small Cell Lung Cancer. Nat Commun 2025, 16, 1477. doi.org/10.1038/s41467-024-55068-2.
(32) Tang, W.; Ehrlich, I.; Wolff, S. B. E.; Michalski, A.-M.; Wolfl, S.; Hasan, M. T.; Luthi, A.; Sprengel, R. Faithful Expression of Multiple Proteins via 2A-Peptide Self-Processing: A Versatile and Reliable Method for Manipulating Brain Circuits. Journal of Neuroscience 2009, 29 (27), 8621-8629. doi.org/10.1523/JNEUROSCI.0359-09.2009.
(33) Niemirowicz, G. T.; Carlevaro, G.; Campetella, O.; Bouvier, L. A.; Mucci, J. A Versatile 2A Peptide-Based Strategy for Ectopic Expression and Endogenous Gene Tagging in Trypanosoma Cruzi. Heliyon 2024, 10 (2), e24595. doi.org/10.1016/j.heliyon.2024.e24595.
(34) Kapp, K.; Schrempf, S.; Lemberg, M. K.; Dobberstein, B. Post-Targeting Functions of Signal Peptides. In Madame Curie Bioscience Database [Internet]; Landes Bioscience, 2013.
(35) Kavanagh, E. W.; Tzeng, S. Y.; Sharma, N.; Cutting, G. R.; Green, J. J. Ligand-Free Biodegradable Poly(Beta-Amino Ester) Nanoparticles for Targeted Systemic Delivery of mRNA to the Lungs. Biomaterials 2025, 313, 122753. doi.org/10.1016/j.biomaterials.2024.122753.
(36) Shen, J.; Lima E Silva, R.; Zhang, M.; Luly, K. M.; Hackett, S. F.; Tzeng, S. Y.; Lowmaster, S. M.; Shannon, S. R.; Wilson, D. R.; Green, J. J.; Campochiaro, P. A. Suprachoroidal Gene Transfer with Nonviral Nanoparticles in Large Animal Eyes. Sci Adv 2024, 10 (10), eadl3576. doi.org/10.1126/sciadv.adl3576.
(37) Vaughan, H. J.; Zamboni, C. G.; Hassan, L. F.; Radant, N. P.; Jacob, D.; Mease, R. C.; Minn, I.; Tzeng, S. Y.; Gabrielson, K. L.; Bhardwaj, P.; Guo, X.; Francisco, D.; Pomper, M. G.; Green, J. J. Polymeric Nanoparticles for Dual-Targeted Theranostic Gene Delivery to Hepatocellular Carcinoma. Sci Adv 8 (29), eabo6406. doi.org/10.1126/sciadv.abo6406.
(38) Son, K. K.; Tkach, D.; Hall, K. J. Efficient in Vivo Gene Delivery by the Negatively Charged Complexes of Cationic Liposomes and Plasmid DNA. Biochimica et Biophysica Acta (BBA)-Biomembranes 2000, 1468 (1-2), 6-10. doi.org/10.1016/S0005-2736 (00) 00311-4.
(39) Souri, M.; Golzaryan, A.; Soltani, M. Charge-Switchable Nanoparticles to Enhance Tumor Penetration and Accumulation. European Journal of Pharmaceutics and Biopharmaceutics 2024, 199, 114310. doi.org/10.1016/j.ejpb.2024.114310.
(40) Salam, M. A.; Al-Amin, M. Y.; Salam, M. T.; Pawar, J. S.; Akhter, N.; Rabaan, A. A.; Alqumber, M. A. A. Antimicrobial Resistance: A Growing Serious Threat for Global Public Health. Healthcare (Basel) 2023, 11 (13), 1946. doi.org/10.3390/healthcare11131946.
(41) Williams, J. A.; Paez, P. A. Improving Cell and Gene Therapy Safety and Performance Using Next-Generation Nanoplasmid Vectors. Molecular Therapy-Nucleic Acids 2023, 32, 494-503. doi.org/10.1016/j.omtn.2023.04.003.
(42) Green, J. J.; Langer, R.; Anderson, D. G. A Combinatorial Polymer Library Approach Yields Insight into Nonviral Gene Delivery. Acc. Chem. Res. 2008, 41 (6), 749-759. doi.org/10.1021/ar7002336.
(43) Karlsson, J.; Rhodes, K. R.; Green, J. J.; Tzeng, S. Y. Poly(Beta-Amino Ester) s as Gene Delivery Vehicles: Challenges and Opportunities. Expert Opin Drug Deliv 2020, 17 (10), 1395-1410. doi.org/10.1080/17425247.2020.1796628.
(44) Leonard, J. P.; Sherman, M. L.; Fisher, G. L.; Buchanan, L. J.; Larsen, G.; Atkins, M. B.; Sosman, J. A.; Dutcher, J. P.; Vogelzang, N. J.; Ryan, J. L. Effects of Single-Dose Interleukin-12 Exposure on Interleukin-12-Associated Toxicity and Interferon-Gamma Production. Blood 1997, 90 (7), 2541-2548.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A composition comprising a nucleic acid comprising a first nucleotide sequence encoding a signal 2 polypeptide and a second nucleotide sequence encoding a signal 3 polypeptide.

2. The composition of claim 1, wherein the first nucleotide sequence is upstream of the second nucleotide sequence.

3. The composition of claim 1, wherein the second nucleotide sequence is upstream of the first nucleotide sequence.

4. The composition of claim 1, further comprising a nucleotide sequence encoding a linker between the first nucleotide sequence and the second nucleotide sequence.

5. The composition of claim 4, wherein the linker is a 2A peptide.

6. The composition of claim 5, wherein the linker is T2A, P2A, E2A, or F2A.

7. The composition of claim 6, wherein the linker further comprises the amino acid sequence GSG at the N-terminus.

8. The composition of claim 1, further comprising a nucleotide sequence encoding a tether between the first nucleotide sequence and the second nucleotide sequence.

9. The composition of claim 8, wherein the tether comprises the amino acid sequence GGGGS (SEQ ID NO: 14).

10. The composition of claim 9, wherein the tether comprises the amino acid sequence (GGGGS)n (SEQ ID NO: 14), where n=2 to 10.

11. The composition of claim 1, wherein the nucleic acid is a plasmid.

12. The composition of claim 1, wherein the nucleic acid is an antibiotic resistance gene-free plasmid.

13. The composition of claim 1, wherein the nucleic acid comprises a bicistronic gene comprising the first nucleotide sequence and the second nucleotide sequence.

14. The composition of claim 1, wherein the signal 2 polypeptide is a cell surface bound protein that regulates immune cells.

15. The composition of claim 1, wherein the signal 3 polypeptide is a secreted protein that regulates immune cells.

16. The composition of claim 1, wherein the signal 3 polypeptide comprises a cytokine.

17. The composition of claim 16, wherein the cytokine comprises an interleukin.

18. The composition of claim 1, wherein the first nucleotide sequence encodes 4-1BBL, CD28, CD80, CD86, OX40L, or GITRL.

19. The composition of claim 1, wherein the second nucleotide sequence encodes TGF-ß1, TGF-ß2, TGF-ß3, TGF-ß4, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IFN-α, or IFN-ß.

20. A gene delivery formulation comprising polymers and the composition of claim 1.

21-51. (canceled)
